SEQUENCE LISTING (40 sequences)

SEQ ID NO: 1 - protein, 523 aa, Cerrena sp.
MSSKLLALITVALVLPLGTDAGIGPVTDLRITNQDIAPDGFTRPAVLAGGTFPGALITGQKGDSFQINVIDELTDASMLTQTSIHWHGFFQKGSAWADGPAFVTQCPIVTGNSFLYDFDVPDQPGTFWYHSHLSTQYCDGLRGPFVVYDPKDPNKRLYDIDNDHTVITLADWYHVLARTVVGVATPDATLINGLGRSPDGPADAELAVINVKRGKRYRFRLVSISCDPNYIFSIDNHSMTVIEVDGVNTQSLTVDSIQIFAGQRYSFVLHANRPENNYWIRAKPNIGTDTTTDSGMNSAILRYNGAPVAEPQTVQSPSLTPLLEQNLRPLVYTPVPGNPTPGGADIVHTLDLSFDAGRFSINGASFLDPTVPVLLQILSGTQNAQDLLPPGSVIPLELGKVVELVIPAGVVGGPHPFHLHGHNFWVVRSAGTDQYNFNDAILRDVVSIGGTGDQVTIRFVTDNPGPWFLHCHIDWHLEAGLAIVFAEGIENTAASNLTPQAWDELCPKYNALSAQKKLNPSTT

SEQ ID NO: 2 - protein, 523 aa, Cerrena sp.
MSSKLLALITVALVLPLGTDAGIGPVTDLRITNQDIAPDGFTRPAVLAGGTFPGALITGQKGDSFQINVIDELTDASMLTQTSIHWHGFFQKGSAWADGPAFVTQCPIVTGNSFLYDFDVPDQPGTFWYHSHLSTQYCDGLRGPFVVYDPKDPNKRLYDIDNDHTVITLADWYHVLARTVVGVATPDATLINGLGRSPDGPADAELAVINVKRGKRYRFRLVSISCDPNYIFSIDNHSMTVIEVDGVNTQSLTVDSIQIFAGQRYSFVLHANRPENNYWIRAKPNIGTDTTTDNGMNSAILRYNGAPVAEPQTVQSPSLTPLLEQNLRPLVYTPVPGNPTPGGADIVHTLDLSFDAGRFSINGASFLDPTVPVLLQILSGTQNAQDLLPPGSVIPLELGKVVELVIPAGVVGGPHPFHLHGHNFWVVRSAGTDQYNFNDAILRDVVSIGGTEDQVTIRFVTDNPGPWFLHCHIDWHLEAGLAIVFAEGIENTAASNPTPQAWDELCPKYNALNAQKKLNPSTT

SEQ ID NO: 3 - protein, 515 aa, Cerrena sp.
MSLLRSLTSLIVLVIGAFAAIGPVTDLHIVNQNLDPDGFNRPTVLAGGTFPGPLIRGNKGDNFKINVIDDLTEHSMLKATSIHWHGFFQKGTNWADGPAFVTQCPITSGNAFLYDFNVPDQAGTFWYHSHLSTQYCDGLRGAFVVYDPNDPNKQLYDVDNGNTVITLADWYHALAQTVTGVAVSDATLINGLGRSATGPANAPLAVISVERNKRYRFRLVSISCDPNFIFSIDHHPMTVIEMDGVNTQSMTVDSIQIFAGQRYSFVMQANQPVGNYWIRAKPNVGNTTFLGGLNSAILRYVGAPDQEPTTDQTPNSTPLVEANLRPLVYTPVPGQPFPGGADIVKNLALGFNAGRFTINGASLTPPTVPVLLQILSGTHNAQDLLPAGSVIELEQNKVVEIVLPAAGAVGGPHPFHLHGHNFWVVRSAGQTTYNFNDAPIRDVVSIGGANDQVTIRFVTDNPGPWFLHCHIDWHLEAGFAVVFAEGINGTAAANPVPAAWNQLCPLYDALSPGDT

SEQ ID NO: 4 - protein, 515 aa, Cerrena sp.
MSLLRSLTSLIVLATGAFAAIGPVTDLHIVNQNLAPDGLNRPTVLAGGTFPGPLIRGNKGDNFKINVIDDLTEHSMLKATSIHWHGFFQKGTNWADGPAFVTQCPITSGNAFLYDFNVPDQAGTFWYHSHLSTQYCDGLRGAFVVYDPNDPNKQLYDVDNGNTVITLADWYHALAQTVTGVAVSDATLINGLGRSATGPANAPLAVISVERNKRYRFRLVSISCDPNFIFSIDHHPMTVIEMDGVNTQSMTVDSIQIFAGQRYSFVMQANQPVGNYWIRAKPNVGNTTFLGGLNSAILRYVGAPDQEPTTDQTPNSTPLVEANLRPLVYTPVPGQPFPGGADIVKNLALGFNAGRFTINGTSFTPPTVPVLLQILSGTHNAQDLLPAGSVIELEQNKVVEIVLPAAGAVGGPHPFHLHGHNFWVVRSAGQTTYNFNDAPIRDVVSIGGANDQVTIRFVTDNPGPWFLHCHIDWHLEAGFAVVFAEGINGTAAANPVPAAWNQLCPLYDALSPGDT

SEQ ID NO: 5 - protein, 278 aa, Cerrena sp.
MSLLRSLTSLIVLATGAFAAIGPVTDLHIVNQNLAPDGFNRPTVLAGGTFPGPLIRGNKGDNFKINVIDDLTEHSMLKATSIHWHGFFQKGTNWADGPAFVTQCPITSGNSFLYDFNVPDQAGTFWYHSHLSTQYCDGLRGAFVVYDPNDPNKQLYDVDNGKTVITLADWYHALAQTVTGVAVSDATLINGLGRSATGPANAPLAVISVERNKRYRFRLVSISCDPNFIFSIDHHPMTVIEMDGVNTQSMTVDSIQIFAGQRYSFVMQANQPVGNYWI

SEQ ID NO: 6 - protein, 246 aa, Cerrena sp.
  misc_feature (60)..(60): Xaa can be any naturally occurring amino acid
AIGPVADLHITDDTIAPDGFSRPAVLAGGGFPGPLITGNKGDAFKLNVIDELTDASMLKXTSIHWHGFFQKGTNWADGPAFVNQCPITTGNSFLYDFQVPDQAGTYWYHSHLSTQYCDGLRGAFVVYDPSDPHKDLYDVDDESTVITLADWYHTLARQIVGVAISDTTLINGLGRNTNGPADAALAVINVDAGKRYRFRLVSISCDPNWVFSIDNHDFTVIEVDGVNSQPLNVDSVQIFAGQRYSF

SEQ ID NO: 7 - protein, 526 aa, Cerrena sp.
MGLNSAITSLAILALSVGSYAAIGPVADIHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 8 - protein, 526 aa, Cerrena sp.
MGLNSAITSLAILALSVGSYAAIGPVADIHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPDDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 9 - protein, 262 aa, Cerrena sp.
AIGPVADLKIVNRDIAPDGFIRPAVLAGGSFPGPLITGQKGNEFKINVVNQLTDGSMLKSTSIHWHGFFQKGTNWADGPAFVNQCPIATNNSFLYQFTSQEQPGTFWYHSHLSTQYCDGLRGPLVVYDPQDPHAVLYDVDDESTIITLADWYHTLARQVKGPAVPGTTLINGLGRHNNGPLDAELAVISVQAGKRQVQFTLFTLYRFRLISISCDPNYVFSIDGHDMTVIEVDSVNSQPLKVDSIQIFAGQRYSFVLNANQP

SEQ ID NO: 10 - protein, 526 aa, Cerrena sp.
MGLNSAITSLAILALSVGSYAAIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 11 - protein, 505 aa, Cerrena sp.
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 12 - protein, 493 aa, Panus rudis
AIGPVTDLHIVNDNIAPDGFTRAAVLAGGTFPGPLITGNKGDNFRIDVIDDLTEESMLKSTSIHWHGFFQKGTNWADGPAFVNQCPITTGHSFLYNFNVPDQAGTFWYHSHLSTQYCDGLRGAFVVYDPNDPHADLYDVDDDSTVITLADWYHTLARQITGVPTPDATLINGLGRSASGPQDSELAVITVEQNKRYRLRLVSISCDPNYIFSIDSHNFTIIEVDGVNSKPLTVDSAQIFAAQRYSVVLNANQPVGNYRIRARPNNGDTTFTNGRNSAILRYKGATVEEPATITPPVSQTPLIEANLKPLASMPVPGTHTPGVADVVKPLQFGFNPPAFTINGASFVPPTVPVLLQILSGAKTAQEIVPSGSIIELPLNSVVELSFPNPTGAPGGPHPFHLHGHTFFVVRSAGQTTYNYDDPIARDVVSTGTAGDNVTIRFATDNAGPWFLHCHIDWHLDAGFAVVMAEGINQTQAANPTPDAWNQLCPTYDAL

SEQ ID NO: 13 - protein, 495 aa, Spongipellis sp.
AVGPVADIHIVDASIAPDGFSRPAVLAGGSFPGPLITGKKGDAFKLNIIDDLTNEDMLKSTSIHWHGFFQKGTNWADGPAFVNQCPITTGNAFLYDFQVPDQAGTYWYHSHLSTQYCDGLRGAFVVYDPVDPHKALYDVDDETTVITLADWYHTLARQIVGVAVADTTLINGLGRNTNGPSDAALAVINVTKGKRYRFRLVSISCDPNYVFSIDGHNMTIIEVDGVNSQPLNVDSIQIFAGQRYSFVLNANQNVGNYWVRANPNLGTTGFTGGINSAILRYQGATVAEPTTAQTTSTNPMQEPNLHPLVPTPVPGLPQAGGVDVVKNLVFGFTGGKFTINGVSFVPLTVPVLLQILSGTTNAQDLLPSGSVIELPLGKTIELTLAAGVLGGPHPFHLHGHNFHVVRSAGQTTANYVNPIVRDVVNTGASPDNVTIRFTTDNPGPWFLHCHIDWHLEAGFAVVFAEGINQTNAANPTPAAWNNLCNLYNALDSGDL

SEQ ID NO: 14 - protein, 487 aa, Curiolus versicolor
GIGPVADLTITNAAVSPDGFSRQAVVVNGGTPGPLITGNMGDRFQLNVIDNLTNHTMLKSTSIHWHGFFQKGTNWADGPAFINQCPISSGHSFLYDFQVPDQAGTFWYHSHLSTQYCDGLRGPFVVYDPNDPAADLYDVDNDDTVITLVDWYHVAANVGPAFPLGADATLINGKGRSPSTTTADLSVISVTPGKRYRFRLVSLSCDPNYTFSIDGHNMTIIETDSINTAPLVVDSIQIFAAQRYSFVLEANQAVDNYWIRANPNFGNVGFTGGINSAILRYDGAAAVEPTTTQTTSTEPLNEVNLHPLVATAVPGSPVAGGVDLAINMAFNFNGTNFFINGASFTPPTVPVLLQIISGAQNAQDLLPSGSVYSLPSNADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDL

SEQ ID NO: 15 - protein, 503 aa, Curiolus versicolor
AIGPVTDLTISNADVTPDGITRAAVLAGGVFPGPLITGNKGDEFQINVIDNLTNETMLKSTTIHWHGIFQAGTNWADGAAFVNQCPIATGNSFLYDFTVPDQAGTFWYHSHLSTQYCDGLRGPLVVYDPDDPNASLYDVDDDTTVITLADWYHTAAKLGPAFPAGPDSVLINGLGRFSGDGGGATNLTVITVTQGKRYRFRLVSISCDPNFTFSIDGHNMTIIEVDGVNHEALDVDSIQIFAGQRYSFILNANQSIDNYWIRAIPNTGTTDTTGGVNSAILRYDTAEDIEPTTNATTSVIPLTETDLVPLDNPAAPGDPQVGGVDLAMSLDFSFNGSNFFINNETFVPPTVPVLLQILSGAQDAASLLPNGSVYTLPSNSIEISFPIITTDGALNAPGAPHPFHLHGHTFSVVRSAGSSTFNYANPVRRDTVSTGNSGDNVTIRFTTDNPGPWFLHCHIDFHLDAGFAIVFAEDTADTASANPVPTAWSDLCPTYDALDSSDL

SEQ ID NO: 16 - protein, 500 aa, Lentinus sp.
AIGPVTDLHIVNSFIQPDGFNRSGVLAEGVFPGPLITGNKGDNFQINVIDELTNGTMLLSTSIHWHGLFQKTTNWADGPAFVNQCPIAANDSFLYNFNVPDQAGTFWYHSHLSTQYCDGLRGPLVVYDPQDPYADLYDVDDDSTVITLADWYHVPAPQAGAVPTSDATLINGLGRSVNGPADAPFAVVNVVQGSRYRFRLVSISCDPNFLFSIDGHTFTVIEADGVNHEPIVADSIQIFAAQRYSFILTANQTADNYWIRANPNNGHTGFAGGINSAILRYSGAPVADPVTTQTSANLLQETSLVPRENPGAPGNATANGVDVDLNLVLSFVGGRFEINGVSFVPPTVPVLLQILSGATTAAELLPSGSVYTLPLNSVIQLSFNTVAVAAVGGPHPFHLHGHTFDVVRSAGSTEYNYINPPRRDVVSTGAATDNVTIRFTTDNAGPWFLHCHIDWHLEAGFAIVFAEDAPDVAAVNPVPDAWNQLCPTYDALTPAQLGGN

SEQ ID NO: 17 - protein, 498 aa, Ceriporiopsis subvermispora
AIGPVTDLEITDAFVSPDGPGLGREAVLAGGTFPGPLIQGNKGDNFQINVVNNLTNHTMLKTTSIHWHGLFQHGTTWADGPAFVSQCPIASGNSFLYNFNVPDQAGTFWYHSHLATQYCDGLRGPLVVYDPNDPHADLYDVDDESTVITLSDWYHAAASTLTFPTFDTTLINGLGRFAGTGGSDSNLTVITVEQGKRYRFRLVSISCDPNWVFSIDQHELTVIEVDGVNAVPLTVDAIQIFAAQRYSFVLNANQTVDNYWIRANPNNGNMGFANGINSAILRYVGADDVEPTSTGTTANLLNEADLSPLVPAAAPGAPNQDFDAVDVPMNLNFTFNGTNLFINGATFVPPSVPVLTQILSGAMTAQELLPAGSVYTLPRNATVQLSLPGNIIAGPHPFHLHGHTFSVIRSAGQSDYNYVDPIQRDVVSIGGATDNVTIRFTTDNPGPWFFHCHIDWHLQAGFAIVFAEETADVASANPVPADWSALCPTYDALSDADH

SEQ ID NO: 18 - protein, 497 aa, Cyathus bulleri
AIGPVLDMHIVNKVISPDGFNRSAVLAGGTADNADFPGPLVTGNKGDHFQLNVIDSLTDTTMLRGTSIHWHGLFQHGTTWADGPVGVNQCPISPGNSFLYDFSVPDQAGTFWYHSHHSTQYCDGLRGPLVVYDPNDPHKSLYDVDDESTVITLADWYHTPAPSAGLVPTTDAVLINGKGRFPTGPTSPLSVINVTPGTKYRFRLVSISCDPNFVFSIDGHTFTIIEVDGVNVTPVEVDSIQIFAGQRYSFVLNANQPVDNYWIRAKPNIAKGVTFDGGINSAILRYAGAPDTDPTTSQTPNSAPMVETDLHPLENPGAPGGSNPADVPLNLAIAFGSNLKFTVNGATFAPPNVPVLLQILSGAQTAQDLLPTGSVYTLPANKVIEISIPGGTTGFPHPFHLHGHTFDVVRSAGSSVYNYDNPVRRDAVNTGGAGDNVTIRFLTDNAGPWILHCHIDWHLELGLAIVFAEDVPTIAASNPPDAWDNLCPAYATQPTGT

SEQ ID NO: 19 - protein, 497 aa, Pycnoporus sanguineus
AIGPVADLTLTNAAVSPDGFSREAVVVNGQTPGPLIAGQKGDRFQLNVIDNLTNHTMLKTTSIHWHGFFQHGTNWADGPAFINQCPIASGHSFLYDFQVPDQAGTFWYHSHLSTQYCDGLRGPFVVYDPNDPQASLYDIDNDDTVITLADWYHVAAKLGPRFPLGADATLINGLGRSPGTTAADLAVIKVTQGKRYRFRLVSLSCDPNHTFSIDGHTMTIIETDSVNTQPLEVDSIQIFAAQRYSFVLDANQPVDNYWIRANPSFGNTGFAGGINSAILRYDGAPEVEPTTNQTTPTKPLNEVDLHPLTPMAVPGLPEPGGVDKPLNMVFNFNGTNFFINGESFVPPSVPVLLQILSGAQAAQDLVPSGSVYVLPSNSTIEISFPATANAPGAPHPFHLHGHTFAVVRSAGSSEYNYDNPIFRDVVSTGTPGDNVTIRFETNNPGPWFLHCHIDFHLDAGFAVVMAEDTPDTAAANPVPQSWSDLCPIYDALDPSDL

SEQ ID NO: 20 - protein, 499 aa, Trametes villosa
GIGPVADLTITNAAVSPDGFSRQAVVVNGGTPGPLITGNMGDRFQLNVIDNLTNHTMVKSTSIHWHGFFQKGTNWADGPAFINQCPISSGHSFLYDFQVPDQAGTFWYHSHLSTQYCDGLRGPFVVYDPNDPAADLYDVDNDDTVITLVDWYHVAAKLGPAFPLGADATLINGKGRSPSTTTADLSVISVTPGKRYRFRLVSLSCDPNYTFSIDGHNMTIIETDSINTAPLVVDSIQIFAAQRYSFVLEANQAVDNYWIRANPNFGNVGFTGGINSAILRYDGAAAVEPTTTQTTSTAPLNEVNLHPLVTTAVPGSPVAGGVDLAINMAFNFNGTNFFINGTSFTPPTVPVLLQIISGAQNAQDLLPSGSVYSLPSNADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDLCPTYDALDPSDQ

SEQ ID NO: 21 - protein, 499 aa, Trametes villosa
AIGPVASLVVANAPVSPDGFLRDAIVVNGVVPSPLITGKKGDRFQLNVVDTLTNHSMLKSTSIHWHGFFQAGTNWAEGPAFVNQCPIASGHSFLYDFHVPDQAGTFWYHSHLSTQYCDGLRGPFVVYDPKDPHASRYDVDNESTVITLTDWYHTAARLGPKFPLGADATLINGLGRSASTPTAALAVINVQHGKRYRFRLVSISCDPNYTFSIDGHNLTVIEVDGINSQPLLVDSIQIFAAQRYSFVLNANQTVGNYWVRANPNFGTVGFAGGINSAILRYQGAPVAEPTTTQTPSVIPLIETNLHPLARMPVPGSPTPGGVDKALNLAFNFNGTNFFINNATFTPPTVPVLLQILSGAQTAQDLLPAGSVYPLPAHSTIEITLPATALAPGAPHPFHLHGHAFAVVRSAGSTTYNYNDPIFRDVVSTGTPAAGDNVTIRFQTDNPGPWFLHCHIDFHLDAGFAIVFAEDVADVKAANPVPKAWSDLCPIYDGLSEANQ

SEQ ID NO: 22 - protein, 499 aa, Trametes sp.
AIGPVASLVVANAPVSPDGFLRDAIVVNGVVPSPLITGKKGDRFQLNVDDTLTNHSMLKSTSIHWHGFFQAGTNWADGPAFVNQCPIASGHSFLYDFHVPDQAGTFWYHSHLSTQYCDGLRGPFVVYDPKDPHASRYDVDNESTVITLTDWYHTAARLGPRFPLGADATLINGLGRSASTPTAALAVINVQHGKRYRFRLVSISCDPNYTFSIDGHNLTVIEVDGINSQPLLVDSIQIFAAQRYSFVLNANQTVGNYWVRANPNFGTVGFAGGINSAILRYQGAPVAEPTTTQTTSVIPLIETNLHPLARMPVPGSPTPGGVDKALNLAFNFNGTNFFINNATFTPPTVPVLLQILSGAQTAQDLLPAGSVYPLPAHSTIEITLPATALAPGAPHPFHLHGHAFAVVRSAGSTTYNYNDPIFRDVVSTGTPAAGDNVTIRFQTDNPGPWFLHCHIDFHLDAGFTIVFAEDVADVKAANPVPKAWSDLCPIYDGLSEANQ

SEQ ID NO: 23 - protein, 504 aa, Trametes sp.
AIGPVTDLTISNADVTPDGITRAAVLAGGVFPGPLITGNKGDEFQINVIDNLTNETMLKSTTIHWHGIFQAGTNWADGAAFVNQCPIATGNSFLYDFTVPDQAGTFWYHSHLSTQYCDGLRGPLVVYDPDDANASLYDVDDDTTVITLADWYHTAAKLGPAFPAGPDSVLINGLGRFSGDGGGATNLTVITVTQGKRYRFRLVSISCDPNFTFSIDGHNMTIIEVGGVNHEALDVDSIQIFAGQRYSFILNANQSIDNYWIRAIPNTGTTDTTGGVNSAILRYDTAEEIEPTTNATTSVIPLTETDLVPLDNPAAPGDPQVGGVDLAMSLDFSFNGSNFFINNETFVPPTVPVLLQILSGAQDAASLLPNGSVYTLPSNSTIEISFPIITTDGALNAPGAPHPFHLHGHTFSVVRSAGSSTFNYANPVRRDTVSTGNSGDNVTIRFTTDNPGPWFLHCHIDFHLDAGFAIVFAEDTADTASANPVPTAWSDLCPTYDALDSSDL

SEQ ID NO: 24 - protein, 499 aa, Ganoderma lucidum
GIGPKADLTISNANIAPDGYTRAAVVVNGVFPGPLITGNKGDRFQLNVIDQLTNHTMLKTTSIHWHGFFQKGTNWADGPAFINQCPIASGHSFLYDFQVPDQAGTFWYHSHLSTQYCDGLRGPFVVYDPKDPLKGLYDVDNDSTVITLSDWYHVAARLGPSFPLGSDSTLINGLGRSTTNATAGLAVINVTQGKRYRFRLVSLSCDPNYTFSIDGHDMSVIEADGIATQPVTANAIQIFSAQRYSFVLTANQTIGNYWIRANPSFGNIGFTNGINSAILRYSGADPIEPTTAQQTTQNLLNEVDLHPFVAKQTPGRATQGGTDVAINMVFNFNGSNFFINNASFTPPTVPVLLQILSGAQAAQDLLPSGSVYTLPINKSIELTFPATVNAPGAPHPFHLHGHSFAVVRSAGSTEYNYNNPVWRDVVSTGTPAAGDNVTIRFQTDNPGPWFLHCHIDFHLEAGFAVVFAEDTADTSLANHVPQAWSDLCPTYDALSADDH

SEQ ID NO: 25 - protein, 499 aa, Curiolus hirsutus
AIGPTADLTISNAEVSPDGFARQAVVVNNVTPGPLVAGNKGDRFQLNVIDNLTNHTMLKSTSIHWHGFFQKGTNWADGPAFVNQCPISSGHSFLYDFQVPDQAGTFWYHSHLSTQYCDGLRGPFVVYDPNDPHASLYDVDNDDTVITLADWYHTAAKLGPAFPLGADATLINGLGRSPSTTAADLAVINVTKGKRYRFRLVSLSCDPNHTFSIDGHDLTIIEVDSINSQPLVVDSIQIFAAQRYSFVLNADQDVGNYWIRANPNFGNVGFAGGINSAILRYDGADPVEPTTTQTTPTKPLNEVDLHPLATMAVPGSPVAGGVDTAINMAFNFNGTNFFINGASFVPPTVPVLLQIISGAQNAQDLLPSGSVYSLPSNADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDLCPIYDALDVNDQ

SEQ ID NO: 26 - protein, 496 aa, Basidiomycete sp.
SIGPVADLTISNGAVSPDGFSRQAILVNDVFPSPLITGNKGDRFQLNVIDNMTNHTMLKSTSIHWHGFFQHGTNWADGPAFVNQCPISTGHAFLYDFQVPDQAGTFWYHSHLSTQYCDGLRGPIVVYDPQDPHKSLYDVDDDSTVITLADWYHLAAKVGPAVPTADATLINGLGRSINTLNADLAVITVTKGKRYRFRLVSLSCDPNHTFSIDGHSLTVIEADSVNLKPQTVDSIQIFAAQRYSFVLNADQDVDNYWIRALPNSGTRNFDGGVNSAILRYDGAAPVEPTTTQTPSTQPLVESALTTLEGTAAPGNPTPGGVDLALNMAFGFAGGRFTINGASFTPPTVPVLLQILSGAQSAQDLLPSGSVYSLPANADIEISLPATSAAPGFPHPFHLHGHTFAVVRSAGSSTYNYANPVYRDVVSTGSPGDNVTIRFRTDNPGPWFLHCHIDFHLEAGFAVVMAEDIPDVAATNPVPQAWSDLCPTYDALSPDDQ

SEQ ID NO: 27 - protein, 497 aa, Rigidoporus microporus
AIGPVADLHISNANISPDGFTRAAVLAGGSFPGPLITGNKGDNFQINVINDLTDADQLKTTTIHWHGFFQHGTNWADGPAFINQCPIASGNSFLYNFQVPDQAGTFWYHSHLSTQYCDGLRGAFVVYDPDDPHASLYDVGDESTVIALADWYHGLARLGPKFPTTNSTLINGLGRYDFGPSSDLAVISVQAGKRYRFRLVSISCDSNYVFSIDEHTFTVIEVDGVNHQPVDADSLQIFAGQRYSIVVNADKSEGGNYWIRASPTPGPQGFANGINSAILRYVGSDEVEPTTNQTTSTNPLVEANLVPLENPGAPGDPTPGGVDVPLNLAVAFDGTGLDFQVNGQTFIPPTVPVLLQILSGAQSATDLLPSGSVYVLPSNATVEISIPAGAVGGPHPIHLHGHTFDVVRSAGSSTYNYVNPPRRDVVSIGNAGDNVTIRFRTDNPGPWFLHCHIDWHLEAGFAVVFAEDIPNVASVNSPPQAWSDLCPIYDALDPSDH

SEQ ID NO: 28 - protein, 501 aa, Polyporus ciliatus
AIGPVADLTITNADINPDGFTRAAVLANNVFPGPLITGNKGDNFQLNVVDNLSNDTMLTATTIHWHGFFQKGTNWADGPAFVNQCPISTGNSFLYNFNAPDQAGTFWYHSHLSTQYCDGLRGPMVVYDDADPHASLYDVDDESTVITLADWYHTAARLGPCFPLGSDSTLINGLGRFAGGDADAPLTVISVTSGKRYRFRLISISCDPNFTFTIQGHTMTVIEVDAVNVQPYEVDSIQIFAGQRYSFVLTADQAVDNYWIQAIPSIGTITTDGGVNSAILRYDGADIVEPAAATVTGSNPLVETSLVPLENLAAPGEPTIGGVDYPLNLDFSFDGTNFAINGATFTPPTVPVLLQIMSGAQDVADLLPSGSIYSLPSNATIELSFPITATNAPGAPHPFHLHGHTFYVVRSAGSTEYNYVNPPQRDTVSTGTAGDNVTIRFTTNNPGPWFLHCHIDFHLEAGFAVVFGEDIPSISDANPPSSAWEDLCPTYNSVYPNGDGN

SEQ ID NO: 29 - protein, 497 aa, Artificial Sequence
  synthetic consensus sequence
AIGPVADLTIXNADVSPDGFTRAAVLAGGVFPGPLITGNKGDRFQLNVIDNLTNHTMLKSTSIHWHGFFQKGTNWADGPAFVNQCPIASGNSFLYDFQVPDQAGTFWYHSHLSTQYCDGLRGPFVVYDPNDPHASLYDVDDDSTVITLADWYHTAARLGPAFPXXADATLINGLGRSXSGPAADLAVINVTQGKRYRFRLVSISCDPNYTFSIDGHNMTVIEVDGVNTQPLTVDSIQIFAAQRYSFVLNANQPVDNYWIRANPNFGTTGFXGGINSAILRYDGAPVVEPTTTQTTSTNPLNETNLHPLVNTAVPGSPTPGGVDVALNLAFNFNGTNFFINGASFTPPTVPVLLQILSGAQNAQDLLPSGSVYTLPSNXTIEISFPATAXAPGAPHPFHLHGHTFAVVRSAGSTTYNYDNPIFRDVVSTGTPGDNVTIRFTTDNPGPWFLHCHIDFHLEAGFAVVFAEDIPDXAAANPVPQAWSDLCPTYDALSPSDX

SEQ ID NO: 30 - protein, 505 aa, Artificial Sequence
  synthetic variant derived from Cerrena unicolor laccase D
AIGPVADLHIVNATLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 31 - protein, 505 aa, Artificial Sequence
  synthetic variant derived from Cerrena unicolor laccase D
AIGPVADLHIVNKDLAPDGVQRPTVLANGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 32 - protein, 505 aa, Artificial Sequence
  synthetic variant derived from Cerrena unicolor laccase D
AIGPVADLHIVNKDLAPDGVQRPTVLAGGTFPGTLITGQKGDNFQLNVTDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 33 - protein, 505 aa, Artificial Sequence
  synthetic variant derived from Cerrena unicolor laccase D
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLANTTVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 34 - protein, 505 aa, Artificial Sequence
  synthetic variant derived from Cerrena unicolor laccase D
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNATPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 35 - protein, 505 aa, Artificial Sequence
  synthetic variant derived from Cerrena unicolor laccase D
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNASDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 36 - protein, 505 aa, Artificial Sequence
  synthetic variant derived from Cerrena unicolor laccase D
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLNASQKVKPKKGTAI

SEQ ID NO: 37 - DNA, 2593 bp, Trichoderma reesie
atggcggacaaggaagcaaccgtcttcatcatcgacctcggcgcgtccatggcagctgtcaatgggggtcgagaagaatccgaccttgattggagcatgagctacgtctgggacaagatcagcaacgtcgtggcctcgaatcgcaagacgctgtgcgttggcgtcgtggggttcagaaccgacgagacaaaccacacgctgagcgaggatgggtacgagaacatctccatattgcagcccctggggccgatgagcatgtccagcctcaaggctcttcagcccaaggtgaagccgagcaggacggtggaaggcgatgccatctcggcgattgtcattgccgtcgacatgattgacaagtacacgaagaagaacaaatggaagcggcagattgttctcattaccgacggccaaggcgagattgatccagatgatattggcgacattgctagaaagatgcgcgactcgaatattgaattgacagtcttgtgagttggcgagaccgtttggcggacggtaatggtgctgacggtgatgcaaggggcgtcgactttgatgctcccgattacggcttcaaagaggaggacaaaccttcagtcaaggtactccatatgttcacttcttttctttttcttctttattttcttttcttttgaagctttcattaacctcttcgttagaagcaaaacgaagagaccctaaaaaagctcgtggatggctgtggcgacgactcaaggttcgcctccatggtcgaggccattgacgacttgaatgagccacgagcaaagtcggtcaagccttacaaaacgtacgaaggtctcttgaccttgggagatccgaaaaacgctcccgcagtggtggaaatccgcgtcgagagatacttcaagacccatctagccaggccacctgccgccagcaccgtggtggtcaaggaggagcaagctgggccgtctcaggcagacgaggacgaacagatggacggagcggaacttacagctgtgaggcaggccaggacatacaaggtcaatgatccagatgcccctggcggtaagcgtgacgttgagtttgagtctctggccaaagggtacgagtacggcaggacggcagtccacatcagcgagtctgatcaaaacgtcaccaagctcgcgacagaaaagagcttcaagatcatcggcttcgtccagaaagaaaaggtattggcttggctctcagcatttgacccgttgctcttggctaacccttgtttagtatgaaatgctccttaatcttggcgaaacctgcgttaccgttgcatccaagtacgatgaaaagtctgagctggcttttagctctctggtgtgggcgctctcggagctcgacgcctacgccgtggcccgcctagtaactaaggaccaaaaggaccccatgctggtgttactgatgccgtatatggagcctgattatgtttgtctctatgatgtgcctctgcctttcgcagaggacatcaggacgtaccagtttcctcccttggacagagtcgttaccgtcagtggccaaacgctcaccaaccatcgcctattgccatccgacgagctcaaccaagcgatgagcgactacgtagatgccatggacatttcaagttatggtatcgatgaagatgggtgagtatagaagatgattgttcaaatctttcacttctaagcattgcttctgatctaggcaaccggctgaatatgccaccatcgatgagttatacaaccctgcgatacatcgcataggccatgcgatcaaacaacgagcgatccacccagagaaacccgtgcccgagatccccccagtcttgcttagattcgcagcacccccgacagaactcgtcgagactgtgcagcctcatatcgatgcactgattcacgctgcagacgtgaagaaaggtactgattccattacatatgcttctctgcacactgatgtttgatttgtgctaacgccccccttagtgccgcccaaggccaagggcaagcgccaaagagaaacagttaaacccatctcgggactggatgtggatgcccttctgggagaagagcagaaaggttccattagtccggagaatgccattccggacttcaaacgagccctcaactcgtccgaagaagtcgagcagattgccgacgccacaaaacaaatgggggccattgtgcggtctctcattacggacagcttcggggatagcaaatatgcccaggcaatggaaggcattggtgcgatgcgtgaggagctgatcaacctggaagagcctggcctgtacaacgactttgtgcgcgacttgaagaaaagtttgctatctggagccttgggtggtgacaggcgagatttctggttcaagatgaggtgggcgaagctgggcctgattgacaagaaacagtcggaggtgtcttcggtcactcttgaggaggcggacgaggtgagtggtgcagcatgctgtcggattatacggacgttgtttgctaacttgtgggatagttttacaagtcgaggtgaggtatctacgttgaccaagaatgggaccatgtatatgagcggtgtaacaacagaatcctgtgctttgagcattgtatga

SEQ ID NO: 38 - DNA, 7685 bp, Artificial Sequence
  synthetic knockout cassette sequence
ggccgcctcaacacccacactcgaggcacacgagttcatcggcggcttcccccacaagctctcggccaacctgctaccggctctctcgcgagacttcccaaagcctacaaacgaggtcgacgtcaaggaggccctcgagcgccagcccggcagatggagcctccagggccagatcaaggccaacaacatgagagcccagagcgccgcactccggctcgacgacaaggagggcaaggcgagagcctttgaggaggccaagcgcgagctactggcgtatcaccacagcgccctgcggaagccttccggcgcaagataatgagcttgatcgcaatgacgagttcacgtacgctttgccatattgttgttgctttttgtttggtcctacatgtacggcgcattggttgggaggatatacccacggagagtgtccgagtggcttctgggatttagagcgtcattagcaggatagagatggttggccaggggaatggaattgacttttcactacaaggaacttgttcactctggtgttgattcccattgcgtgactggtagtagggaggaatgcttttactttgtgccactagaccgcagagaagggttggttgcaagcggggtccgtgtataccgaccaagagtgatgggcatacagcaacgtttctgaacgacttcattttgtccgagtctactggatgcgagatgccagcgtgaagccgtacgccaccagggcgacgaactcgacaaggttgacgagggaggagatgccgtgcagcatgccaaacttcttgttgagggcacgcatctcatccgactgtgcatccttgtcataccactcctttccgtctcgcttggctggtgggagggttcaacaaatccatcgtcagccatccggggtctcaaatcaatggcgtgcatgcggagtcgggcttgaggctaaccttgtccatggcggtccttcatggtcttgacagtggcgggaagcagcacggcgaggttgacgaggccgctgacgaacatggttgcgatgggcaccaaggagctccacttgttgggagcgtcgacgaggccgccgatgccgcccttgatgcccaagagggcgtttccggggaacgtgagggcgagcagcgcggggatggccgtctgcatgccaaagtagatggggaacagcttgctctggatggcggagaaggagggccggctgacggtgcggaacatgacgatgccgttgacgaaggactgcagtagcgtagtgtgatggtaagcagctggccggcgcgcctgagacaatggccggcaatggtaaaaaggaccaagatgtactaggtagttgcaatgtggcttattacctacctactacctggtaggcacctactaggtacttgggtagacggacaatgaaatttgaagtcggggttgcaggaaagcagggcgctggacacattgtgcttcaggcggtacccgtcgtcatcgtcagccaatgtcgaggcccggcagcccgaggagcgagacaaccttggccggaggagcccgcaggtacctgccaaagcgcggctggtacctctcaaccctctcaggcctgttggatgccctatgacatgccctgggggatgcagctgttgccccggccccgcactttcgggtgaccgcgaggctgctgattggctggttgccacgggctgggcggtccctgaagttgttgccatctgaactctgtcggcgctggcgtcggctgcgcccaatgggaggcgagacaactcagggtactagaatcactgacagaagaagagaatcgaaagtaggtagacagccaattcgttgcatggcaggcaaccgcacaggagaaaaattgactaccccacaatcaggcacagtaagtagggcacagtacgtatgtacagacaaggcgcaagcgatactgcgcgacccggtacctcgccggcttgacacgtgcgacaggctactttactagtattcgcagcggcgggtcgcgcattattacatgtactgtgccgccatttgatgactgggctgctgcagtattagtagatctgcccggcatcgcccttccatgggcgcgacccgggactggaccctctgactctacctacatgtacctaggccgggccgggcttggtgacttttgtccgatcaggtcgttcgcctggctacctattatttctctttcttcttctccatcctgcttctggccttgcaattcttcttcgccactcctccctcttccccccgcgatacccttgaattcgtcagagaggaaaagacgagaaaaaaaagggcagcagagacgtcggtctggctcacgtgctgcatctctgcgcactctcattttttttattgtccgacccctccctcaaccttctccttcgttgacaggctaagccttgcttcgacgctctctctttgaatttttctacttctaccttcttttcttgcgtgttacccaccatagctcgattcacgatgctccgaagtcgccaagtcacagccagggccgtccgggctctgggccaggcgcgcgcctttacctcgacgaccaagcctgtcatgatccagagcagccagaggaaacaggccaacgccagcgctgctccgtaagtcgcccattgccattgcatcttctgtttgatatatacttcctgctgcttgcgtggcgtcgtctctcggttatgcgtgtcaaggaccaggtgtgttcgcatcgtggttttccagcgccgattaccgggggacgaatttttggctgctcaactcgcgcgcgcgcattctgattcttcgttttcaatcttgagcgacaactggctaacataatggccattggcaattgcttcacacagacaagtccgccctgtaccgagccctgctttcaacgctgaagacaaagaccgcagccatgtgcagcctctggtcaacccgtcgaagcccgacatggatgaatcgtatgtccacgtcccctcgtcccgccctacaaaatgaacacgattacaccagaatttttgcaacaatcgacacttctataacagaccaattgagctttgttctgaccaatcatgttgctctagattcattggcaaaaccggaggcgaaatcttccacgagatgatgctgcgacagggtgtcaagcacatttgtaggttccgatgccggccgcccacacgggctccatccttgctccatctctccagctaggcaaatctcgctaaccttgagtcaccatccagtcggataccctggcggcgctatcctgcccgtcttcgacgccatctacaactcaaaacacttcgacttcatcctgccccgtcatgagcagggagctggccatatggccgagggctatgcccgtgcctcgggcaaacccggtgtcgtcctggtgacttccggccccggtgctaccaatgtcatcacgcccatgcaggatgccctgtcggacggaacgcccttggtcgtcttctgcggccaggtccccaccacggccatcggcagcgatgacttccaagaggccgacgtcgtgggcatctcgcgggcctgcaccaagtggaacgtcatggtcaagagcgttgctgagctgccgcggagaatcaacgaggcctttgagattgccaccagcggccgccctggccccgtcctcgtcgacctgcccaaggatgtcacggctggtatcctgaggagagccatccctacggagactgctctgccgtctctgcccagtgccgcctcccgcgccgccatggagctgagctccaagcagctcaacgcctccatcaagcgtgccgccgacctcatcaacatcgccaagaagcccgtcatctacgccggtcagggtgtcatccagtccgagggcggcgttgagctcctgaagcagctggcggacaaggcctccatccccgtcaccaccaccctccatggcctgggtgcctttgatgagctggacgagaagtcgctgcacatgctgggcatgcacggctcggcgtatgccaacatggccatgcagcaggccgacctcatcatcgccctcggcagccgattcgacgaccgtgttactctgaatgtctccaaatttgcgcctgcagccaggcaagctgctgccgagggccgcggcggcatcattcactttgagatcatgcccaagaacatcaacaaggtcatccaggcgaccgaggccgtcgagggcgacgtcgccaccaacctgaagcacctcattccccagattgccgaaaagtccatggcggaccgaggagagtggttcggcctcatcaatgagtggaagaagaagtggcccctgtcaaactaccagcgcgcggagcgggctggcctcatcaagccgcagacggtcatggaggagattagcaacctgacggccaaccgaaaggacaagacgtacattgccacgggtgtcggccagcaccagatgtgggttgcccagcacttccgctggaggcaccctcgatccatgattacctctggtggtctgggcaccatgggctacggtctccccgcggccattggcgccaaggtggcccagcccgacgctctcgtaattgacgttgatggcgatgcctcgtttaacatgacgctgacggagctgtcgactgctgcacagttcaacattggcgtcaaggtggttgtgctcaacaacgaggagcagggcatggtgacgcagtggcagaacctcttttacgaggaccgatatgcccacacgcaccagaagaaccccgacttcatgaagctggccgacgccatgggcgttcagcaccagcgcgtgacggagccggagaagctggtcgatgccctgacgtggctgatcaacaccgatggcccggccctgttggaggttgtcacggacaagaaggtgcctgtcctgcccatggtgcccgccggatcggccctgcacgagttcctcgtctttgaacctggtgagtctacttcagacatattgcttgcgcattgcagatactaacactctcacagaaaaggataagcagcgccgtgagctgatgaaggagagaacaaagggtgtgcactcctaaagcgatgatgtctgcgaggggttcttcgttgaaccctagttcaggcaccatcttaccctcttattttttcccgtgggctttcattttgtgtcatccgagcatgacgttgtagggttggagtttcttcctttttatcttgtcatttactggtacccataggcgcgagactaggcttccatgttttgttttgcgactttcaaaaagtacttttagtggtttggggcacgacgagggggggcaacctcttctgtcgaaaaaggtggctggatggatgagatgagatgagatgagggtgaagatagatacctgcagtgtttttgacgcgacgggatggcgatcgcagcacccccgacagaactcgtcgagactgtgcagcctcatatcgatgcactgattcacgctgcagacgtgaagaaaggtactgattccattacatatgcttctctgcacactgatgtttgatttgtgctaacgccccccttagtgccgcccaaggccaagggcaagcgccaaagagaaacagttaaacccatctcgggactggatgtggatgcccttctgggagaagagcagaaaggttccattagtccggagaatgccattccggacttcaaacgagccctcaactcgtccgaagaagtcgagcagattgccgacgccacaaaacaaatgggggccattgtgcggtctctcattacggacagcttcggggatagcaaatatgcccaggcaatggaaggcattggtgcgatgcgtgaggagctgatcaacctggaagagcctggcctgtacaacgactttgtgcgcgacttgaagaaaagtttgctatctggagccttgggtggtgacaggcgagatttctggttcaagatgaggtgggcgaagctgggcctgattgacaagaaacagtcggaggtgtcttcggtcactcttgaggaggcggacgaggtgagtggtgcagcatgctgtcggattatacggacgttgtttgctaacttgtgggatagttttacaagtcgaggtgaggtatctacgttgaccaagaatgggaccatgtatatgagcggtgtaacaacagaatcctgtgctttgagcattgtatgatatgattattgatgaaccggacaaaagggggtaggggattgatgccatcacgaccgattgaccagacctggattctcgcacagcatggctgctgattttgttgaccttgcgacgtaacatccctgaagaacaacctactattaacctatcatttagcagaagctctgtaaccttcttgattcttgtattcagcttctgagtctgtcaaatgtaatcatttcgaggttgtgtaattccggccaagcaggcggccgtctgccagcgcctgcctaggctgcaccgcaatctgcccaatcagctgcccttcagtttcgtttgaccttgcagctgcccttcatcctttatctgcacacaattctttttcctctgctctgcgcattcttctctctctcgtctcccttctcaagctcaacttcacctcatccgctccactacaagccctcccgtcgtcgtctcgcatcctcatctcgactgcggccagcaaaacaagcaaagccgtgatcgatcctcagcatggctaccttcaacctcaccgtccgcctggagatgctcaaagaaattggaatcaccgtccaatacggcgagcatgtagcgaaagaagcagccagcaacgaagcagcgatggcattcgaagaagaagaagagttccccgccgttgtgccgcccaaggcagaacagcacgcctctgaacacgacgctggccacgatgcttgggacgcggctgcccacatctcgacttcggcgcaagaacagcagaagccccaggagatggacgactcgtctatcgtgatgccgctggactactccaagtttgtcgttggagagcctgcggacgaatccatcagcttttgctcgtggaaggtcgtcgaggcttatcctgaccagtttatcggcaaggcaaacaggcctcgtgtatgtagcgattgctttctctgcattatgggaatctcaagagagtatggtagaagataactgacaacttgcaggccaagccgtactttgacaagattttggaagacagagtctgggatttgtgaggatcttgattgatgtgcatatggcgacatgcctgctaatatcattgtagcttctatctctacaaccccgagaagccttcagagaagcctcgcgtgctggtgcccactgttcagctcgaaggctttctcaaaagcatcaacagagcgctcggtacttctctcaccattccaggaggggcaaaccaggaccgtttttatctgaggttcggccagggagacaccccaaggcctcgatatctacagaggtcgagagaccagaaatccctaaagattgaaacgttccccgattttcaacaggcggactacgacagctttaggaacgcgcatggcgccatccaggaggactggttgaagaactggcagatgctggtacctcggccgagtttcgacaagaagaaaaatgcagacaaaagagcagccaagagaaggctcgagcgagagcgaatgcttcacaatacgcaggaatttcttcatttggcaggtaagggcaaaggggctgacgtgg

SEQ ID NO: 39 - DNA, 9259 bp, Artificial Sequence
  synthetic knockout cassette sequence
atcacgccctcgcataaaagaccctcaagagtccatgtgccctatctgcctgatcttcctaacccttatttaacattggccctatcacaacctagttcttcttcagcctgctttgtcaacacttgtcacggttcaactcaacgtaatcagcaggtagcaggacgaggatagggagagaaacgaagagaagaagaggagagaggaagaaaaaaaaaagaaaagaaagaaaaagggaaaaggaaagaaggaggaaaagagaagaaagtcagatgaagaagcaagaagacgccatggtagccaccgcttgtcagggctccttagcaacgaacaactctagcttggggacttgtcgatgtgtcgtttccttcctacccatcagcaccaacgatgagttcgatatagacgaggacctcatggaagtagagaccattgggttcgacaggatctctcagtttcacttctatgaggtctgtcgctcggatgactttttgaggagcttccccttctgcttcaaccccaaactctctttcctgaaaccgcagcacgttggcacggccgtgttgctggagcagtttgctttcgagcactctcagcgtggtttcagcagcccactggtgagtggcctcctttgacgtccacaccttgctcctgtcgcatgcgtatctggtgggaacgactgctccaaggaggattgctaacgaggttgtaggccgaatatcgcatcagattctccggtaaccttagctacggcctcttcaacatctgtgacatgacggagcgcaagtactggtggttggcgaccaagatgcgcggctggaacatcgacggctgccccgaagacgtcaggagactcatgtttgttcacatcatcgccaccctgggatgcagccccgtcgtgacggatgaagacatggactaccccaagaactgggcggcaattctccacggtagagacagatatccgagtgaacctgtgggccaccggcctcatgggcgcaccatctgcctccactcggtggccgtctgccctcgtctccagggcttgggtctcggtactgcgactctgaagtcgtatgtgcagcgcatgaacagcctcggcgccgcggaccgtgttgctctcgtttgccgcaagcccgagacgagattttttgaaagatgcggcttcaggaacagcggccggagtagtatcaagactctggtcggcgaatactacaacatggtgtgtgcttccacatcgacttggccagactctatacgattttcaaacctcgctatacgtcatattgacttgtttctttaggtcttcgatttgcccgggcccaaagactttatcgactggaatagcattgccgacgctgccaagaagatgtgaaccatttgactgatacgatgtgtgctacgcatgtcgaccttctttgtttgtttctttggcggctctttgtataccttgggacacggcagacgcatgtctatgtgaagaaaacgttcacggcgctgtttgcatcaggaatatgatcattaaacatggagcgtaatggtattaatgatcaactagaaaaatggtatggaagggcgagagggcgatcaacaaagcagcccggggcatagtctggaagcagcaggaattggaagggaaaaggaagctgcacaatgaagggatatcgtgagcggagtggctcacgagagtatcaacagactggcgaaagcaagcaattgccaacgccggctattaggccataagatggcctgttgtgagtcccagttgcacgtatccccatatgactgctctgtcgctgacttgaaaaaaaatagggaggataaaggagaaagaaagtgagacaacccgtgagggacttggggtagtaggagaacacatgggcaaccgggcaatacacgcgatgtgagacgagttcaacggcgaatggaaaatcttgaaaaacaaaataaaataactgccctccatacgggtatcaaattcaagcagttgtacggaggctagctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacacctcgtgctagactaggcgcgccaggaagcccggaaggtaagtggattcttcgccgtggctggagcaaccggtggattccagcgtctccgacttggactgagcaattcagcgtcacggattcacgatagacagctcagaccgctccacggctggcggcattattggttaacccggaaactcagtctccttggccccgtcccgaagggacccgacttaccaggctgggaaagccagggatagaatacactgtacgggcttcgtacgggaggttcggcgtagggttgttcccaagttttacacaccccccaagacagctagcgcacgaaagacgcggagggtttggtgaaaaaagggcgaaaattaagcgggagacgtatttaggtgctagggccggtttcctccccatttttcttcggttccctttctctcctggaagactttctctctctctcttcttctcttcttccatcctcagtccatcttcctttcccatcatccatctcctcacctccatctcaactccatcacatcacaatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagactagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtagtgattttaccgctgatgaaatgactggactccctcctcctgctcttatacgaaaaattgcctgactctgcaaaggttgtttgtcttggaagatgatgtgcccccccatcgctcttatctcataccccgccatctttctagattctcatcttcaacaagaggggcaatccatgatctgcgatccagatgtgcttctggcctcatactctgccttcaggttgatgttcacttaattggtgacgaattcagctgatttgctgcagtatgctttgtgttggttctttccaggcttgtgccagccatgagcgctttgagagcatgttgtcacttataaactcgagtaacggccacatattgttcactacttgaatcacatacctaattttgatagaattgacatgtttaaagagctgaggtagctttaatgcctctgaagtattgtgacacagcttctcacagagtgagaatgaaaagttggactccccctaatgaagtaaaagtttcgtctctgaacggtgaagagcatagatccggcatcaactacctggctagactacgacgtcaattctgcggccttttgacctttatatatgtccattaatgcaatagattctttttttttttttttttttttttttttttttttttttttttgcccaatttcgcagatcaaagtggacgttatagcatcataactaagctcagttgctgagggaagccgtctactaccttagcccatccatccagctccataccttgatactttagacgtgaagcaattcacactgtacgtctcgcagctctccttcccgctcttgcttccccactggggtccatggtgcgtgtatcgtcccctccacaattctatgccatggtacctccagcttatcaatgccccgctaacaagtcgcctctttgccttgatagcttatcgataaaactttttttccgccagaaaggctccgcccacagacaagaaaaaaaattcaccgcctagcctttggccccggcatttggctaaacctcgagcctctctcccgtcttggggtatcaggaagaaaagaaaaaaatccatcgccaagggctgttttggcatcaccacccgaaaacagcacttcctcgatcaaaagttgcccgccatgaagaccacgtggaaggacatccctccggtgcctacgcaccaggagtttctggacattgtgctgagcaggacccagcgcaaactgcccactcagatccgtgccggcttcaagattagcagaattcgaggtacgtcgcattgcccatcgcaggatgtctcattatcggggtccttggagaacgatcatgattgcatggcgatgctaacacatagacagccttctacactcgaaaggtcaagttcacccaggagacgttttccgaaaagttcgcctccatcctcgacagcttccctcgcctccaggacatccaccccttccacaaggaccttctcaacaccctctacgatgccgaccacttcaagattgcccttggccagatgtccactgccaagcacctggtcgagaccatctcgcgcgactacgtccgtctcttgaaatacgcccagtcgctctaccagtgcaagcagctcaagcgggccgctctcggtcgcatggccacgctggtcaagcgcctcaaggaccccctgctgtacctggaccaggtccgccagcatctcggccgtcttccctccatcgaccccaacaccaggaccctgctcatctgcggttaccccaatgttggcaagtccagcttcctgcgaagtatcacccgcgccgatgtggacgtccagccctatgctttcaccaccaagagtctgtttgtcggccactttgactacaagtacctgcgattccaggccattgatacccccggtattctggaccaccctcttgaggagatgaacactatcgaaatgcagaggtatgtggcgcggcta

SEQ ID NO: 40 - DNA, 9088 bp, Artificial Sequence
  synthetic knockout cassette sequence
atcacgccctcgcataaaagaccctcaagagtccatgtgccctatctgcctgatcttcctaacccttatttaacattggccctatcacaacctagttcttcttcagcctgctttgtcaacacttgtcacggttcaactcaacgtaatcagcaggtagcaggacgaggatagggagagaaacgaagagaagaagaggagagaggaagaaaaaaaaaagaaaagaaagaaaaagggaaaaggaaagaaggaggaaaagagaagaaagtcagatgaagaagcaagaagacgccatggtagccaccgcttgtcagggctccttagcaacgaacaactctagcttggggacttgtcgatgtgtcgtttccttcctacccatcagcaccaacgatgagttcgatatagacgaggacctcatggaagtagagaccattgggttcgacaggatctctcagtttcacttctatgaggtctgtcgctcggatgactttttgaggagcttccccttctgcttcaaccccaaactctctttcctgaaaccgcagcacgttggcacggccgtgttgctggagcagtttgctttcgagcactctcagcgtggtttcagcagcccactggtgagtggcctcctttgacgtccacaccttgctcctgtcgcatgcgtatctggtgggaacgactgctccaaggaggattgctaacgaggttgtaggccgaatatcgcatcagattctccggtaaccttagctacggcctcttcaacatctgtgacatgacggagcgcaagtactggtggttggcgaccaagatgcgcggctggaacatcgacggctgccccgaagacgtcaggagactcatgtttgttcacatcatcgccaccctgggatgcagccccgtcgtgacggatgaagacatggactaccccaagaactgggcggcaattctccacggtagagacagatatccgagtgaacctgtgggccaccggcctcatgggcgcaccatctgcctccactcggtggccgtctgccctcgtctccagggcttgggtctcggtactgcgactctgaagtcgtatgtgcagcgcatgaacagcctcggcgccgcggaccgtgttgctctcgtttgccgcaagcccgagacgagattttttgaaagatgcggcttcaggaacagcggccggagtagtatcaagactctggtcggcgaatactacaacatggtgtgtgcttccacatcgacttggccagactctatacgattttcaaacctcgctatacgtcatattgacttgtttctttaggtcttcgatttgcccgggcccaaagactttatcgactggaatagcattgccgacgctgccaagaagatgtgaaccatttgactgatacgatgtgtgctacgcatgtcgaccttctttgtttgtttctttggcggctctttgtataccttgggacacggcagacgcatgtctatgtgaagaaaacgttcacggcgctgtttgcatcaggaatatgatcattaaacatggagcgtaatggtattaatgatcaactagaaaaatggtatggaagggcgagagggcgatcaacaaagcagcccggggcatagtctggaagcagcaggaattggaagggaaaaggaagctgcacaatgaagggatatcgtgagcggagtggctcacgagagtatcaacagactggcgaaagcaagcaattgccaacgccggctattaggccataagatggcctgttgtgagtcccagttgcacgtatccccatatgactgctctgtcgctgacttgaaaaaaaatagggaggataaaggagaaagaaagtgagacaacccgtgagggacttggggtagtaggagaacacatgggcaaccgggcaatacacgcgatgtgagacgagttcaacggcgaatggaaaatcttgaaaaacaaaataaaataactgccctccatacgggtatcaaattcaagcagttgtacggaggctagatagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacacctcgtgctagactaggcgcgtcaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgtacgcgtttcgggtttacctcttccagatacagctcatctgcaatgcattaatgcattggacctcgcaaccctagtacgcccttcaggctccggcgaagcagaagaatagcttagcagagtctattttcattttcgggagactagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtagtgattttaccgctgatgaaatgactggactccctcctcctgctcttatacgaaaaattgcctgactctgcaaaggttgtttgtcttggaagatgatgtgcccccccatcgctcttatctcataccccgccatctttctagattctcatcttcaacaagaggggcaatccatgatctgcgatccagatgtgcttctggcctcatactctgccttcaggttgatgttcacttaattggtgacgaattcagctgatttgctgcagtatgctttgtgttggttctttccaggcttgtgccagccatgagcgctttgagagcatgttgtcacttataaactcgagtaacggccacatattgttcactacttgaatcacatacctaattttgatagaattgacatgtttaaagagctgaggtagctttaatgcctctgaagtattgtgacacagcttctcacagagtgagaatgaaaagttggactccccctaatgaagtaaaagtttcgtctctgaacggtgaagagcatagatccggcatcaactacctggctagactacgacgtcaattctgcggccttttgacctttatatatgtccattaatgcaatagattctttttttttttttttttttttttttttttttttttttttttgcccaatttcgcagatcaaagtggacgttatagcatcataactaagctcagttgctgagggaagccgtctactaccttagcccatccatccagctccataccttgatactttagacgtgaagcaattcacactgtacgtctcgcagctctccttcccgctcttgcttccccactggggtccatggtgcgtgtatcgtcccctccacaattctatgccatggtacctccagcttatcaatgccccgctaacaagtcgcctctttgccttgatagcttatcgataaaactttttttccgccagaaaggctccgcccacagacaagaaaaaaaattcaccgcctagcctttggccccggcatttggctaaacctcgagcctctctcccgtcttggggtatcaggaagaaaagaaaaaaatccatcgccaagggctgttttggcatcaccacccgaaaacagcacttcctcgatcaaaagttgcccgccatgaagaccacgtggaaggacatccctccggtgcctacgcaccaggagtttctggacattgtgctgagcaggacccagcgcaaactgcccactcagatccgtgccggcttcaagattagcagaattcgaggtacgtcgcattgcccatcgcaggatgtctcattatcggggtccttggagaacgatcatgattgcatggcgatgctaacacatagacagccttctacactcgaaaggtcaagttcacccaggagacgttttccgaaaagttcgcctccatcctcgacagcttccctcgcctccaggacatccaccccttccacaaggaccttctcaacaccctctacgatgccgaccacttcaagattgcccttggccagatgtccactgccaagcacctggtcgagaccatctcgcgcgactacgtccgtctcttgaaatacgcccagtcgctctaccagtgcaagcagctcaagcgggccgctctcggtcgcatggccacgctggtcaagcgcctcaaggaccccctgctgtacctggaccaggtccgccagcatctcggccgtcttccctccatcgaccccaacaccaggaccctgctcatctgcggttaccccaatgttggcaagtccagcttcctgcgaagtatcacccgcgccgatgtggacgtccagccctatgctttcaccaccaagagtctgtttgtcggccactttgactacaagtacctgcgattccaggccattgatacccccggtattctggaccaccctcttgaggagatgaacactatcgaaatgcagaggtatgtggcgcggct

(19) United States
(12) Patent Application Publication
(10) Pub. No.: US 2014/0123404 A1
(43) Pub. Date: May 8, 2014

(54) LACCASE VARIANTS HAVING INCREASED EXPRESSION AND/OR ACTIVITY

(71) Applicants: Huaming Wang, Fremont, CA (US); Michael Ward, San Francisco, CA (US)

(72) Inventors: Huaming Wang, Fremont, CA (US); Michael Ward, San Francisco, CA (US)

(73) Assignee: DANISCO US INC., Palo Alto, CA (US)

(21) Appl. No.: 14/110,123

(22) PCT Filed: Mar. 20, 2012
(86) PCT No.: PCT/US12/29786

§ 371 Date: Dec. 2, 2013

Related U.S. Application Data
(60) Provisional application No. 61/472,568, filed on Apr. 6, 2011.

(51) Int. Cl. C12N 9/02; D06L 3/11

(52) U.S. Cl. CPC C12N 9/0061; D06L 3/11
USPC 8/401; 435/189

(57) ABSTRACT

The present compositions, methods, and systems, relating to variant laccase enzymes that demonstrate increased expression and/or activity compared to a reference parental laccase enzyme. The variant enzymes include mutations that affect glycosylation, surface charge, or surface hydrophobicity, resulting in improved enzyme expression and/or enzyme activity.

### PRIORITY

The present application claims priority to U.S. Provisional Application Ser. No. 61/472,568, filed on Apr. 6, 2011, which is hereby incorporated by reference in its entirety.

### TECHNICAL FIELD

The present compositions, methods, and systems, relating to variant laccase enzymes that demonstrate increased expression and/or activity compared to a reference parental laccase enzyme. The variant enzymes include mutations that affect glycosylation, surface charge, or surface hydrophobicity, resulting in improved enzyme expression and/or enzyme activity.

### BACKGROUND

Laccases are copper-containing phenol oxidizing enzymes that are known to be good oxidizing agents in the presence of oxygen. Laccases are found in microbes, fungi, and higher organisms. Laccase enzymes are used for many applications, including pulp and paper bleaching, treatment of pulp waste water, de-inking, industrial color removal, bleaching in laundry detergents, oral care teeth whiteners, and as catalysts or facilitators for polymerization and oxidation reactions.

Laccases can be utilized for a wide variety of applications in a number of industries, including the detergent industry, the paper and pulp industry, the textile industry and the food industry. In one application, phenol oxidizing enzymes are used as an aid in the removal of stains, such as food stains, from clothes during detergent washing. Most laccases exhibit pH optima in the acidic pH range while being inactive in neutral or alkaline pHs.

Laccases are known to be produced by a wide variety of fungi, including species of the genera Aspergillus, Neurospora, Podospora, Botrytis, Pleurotus, Fornes, Phlebia, Trametes, Polyporus, Stachybotrys, Rhizoctonia, Bipolaris, Curvularia, Amerosporium, Lentinus, Myceliophtora, Coprinus, Thielavia, Cerrena, Streptomyces, and Melanocarpus. For many applications, the oxidizing efficiency of a laccase can be improved through the use of a mediator, also known as an enhancing agent.

Despite the availability of a wealth of microbial expression systems, laccases are difficult to express in culture at high levels. Laccases with high specific activity can be particularly difficult to express, in some cases at a level less than 1 g/L, presenting an impediment to their large scale production.

### SUMMARY

Described are compositions, methods, and systems, relating to variant laccase enzymes that demonstrate increased expression and/or activity compared to a reference parental laccase enzyme.

In one aspect, a variant laccase enzyme derived from a parental laccase enzyme is provided, the variant laccase enzyme having: (a) a mutation at a position corresponding to position 68 of the amino acid sequence of SEQ ID NO: 11; (b) a mutation that alters the surface charge of the parental laccase enzyme; (c) a mutation that alters the surface hydrophobicity of the parental laccase enzyme; and/or (d) a mutation at an amino acid position corresponding to a non-conservative, hydrophobic amino acid residue located on the surface of the parental laccase enzyme; wherein the mutation is a substitution to a different amino acid residue compared to the parental laccase.

In some embodiments, the variant laccase enzyme has a mutation at a position corresponding to position 68 of the amino acid sequence of SEQ ID NO: 11, wherein the mutation is a substitution of an aromatic amino acid residue to a non-aromatic amino acid residue. In some embodiments, the mutation is a substitution of an aromatic amino acid residue to an aliphatic amino acid residue. In some embodiments, the mutation is a substitution of an aromatic amino acid residue to A, V, L, or I. In some embodiments, the mutation is equivalent to F68L in SEQ ID NO: 11.

In some embodiments, the variant laccase enzyme has a mutation that alters the surface charge or alters the surface hydrophobicity of the parental laccase enzyme, wherein the mutation is at a position equivalent to position 130, 265, 287, 293, or 319, in SEQ ID NO: 11.

In some embodiments, the variant laccase enzyme has a mutation that alters the surface charge or alters the surface hydrophobicity of the parental laccase enzyme, wherein the mutation is at a position equivalent to position 130 in SEQ ID NO: 11.

In some embodiments, the variant laccase enzyme has a mutation that alters the surface charge or alters the surface hydrophobicity of the parental laccase enzyme, wherein the mutation is at: (a) an amino acid position equivalent to position 130 in SEQ ID NO: 11, wherein the residue in the parental laccase is substituted with a different residue selected from D, E, R, and K; (b) an amino acid position equivalent to position 265 in SEQ ID NO: 11, wherein the residue in the parental laccase is substituted with a different residue selected from R, H, and V; (c) an amino acid position equivalent to position 287 in SEQ ID NO: 11, wherein the residue in the parental laccase is substituted with a different residue selected from P, H, and G; (d) an amino acid position equivalent to position 293 in SEQ ID NO: 11, wherein the residue in the parental laccase is substituted with a different residue selected from N, T, and S; and/or (e) an amino acid position equivalent to position 319 in SEQ ID NO: 11, wherein the residue in the parental laccase is substituted with a different residue selected from W, T, and S.

In some embodiments, the variant laccase enzyme has mutations equivalent to: (a) I265R/V287G, (b) I265R/V293T; (c) I265R/V319T; (d) I265R/V287G/V319T; (e) I265R/V287G/V293T/V319T; (f) I265R/V287P; (g) I265R/N335R; (h) I265R/N130E; (i) F68L/I265R; (j) F68L/I265R/V287G; (k) F68L/I265R/V293T; (l) F68L/I265R/V319T; (m) F68L/I265R/V287G/V319T; (n) F68L/I265R/V287G/V293T/V319T; (o) F68L/I265R/V287P; (p) F68L/I265R/N335R; or (q) F68L/I265R/N130E; in SEQ ID NO: 11.

In some embodiments, the parental laccase is obtainable from a Cerrena species. In some embodiments, the parental laccase is obtainable from Cerrena unicolor. In some embodiments, the parental laccase is laccase D from C. unicolor.

In some embodiments, the parental laccase has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

In some embodiments, any of the variant laccase enzymes described herein have an amino acid sequence that is at least 70% identical to the amino acid sequence of SEQ ID NO: 11. In some embodiments, any of the variant laccase enzymes described herein has an amino acid sequence that is at least 80% identical to the amino acid sequence of SEQ ID NO: 11. In some embodiments, any of the variant laccase enzymes described herein has an amino acid sequence that is at least 90% identical to the amino acid sequence of SEQ ID NO: 11. In some embodiments, any of the variant laccase enzymes described herein has an amino acid sequence that is at least 95% identical to the amino acid sequence of SEQ ID NO: 11. In some embodiments, any of the variant laccase enzymes described herein has an amino acid sequence that is at least 96%, at least 97%, at least 98%, or even at least 99% identical to the amino acid sequence of SEQ ID NO: 11.

In some embodiments, any of the variant laccase enzymes described herein further comprises a mutation that introduces a glycosylation site into the amino acid sequence of the parental laccase.

In another aspect, a composition comprising one or more of any of the variant laccase enzymes described herein is provided. In some embodiments, the composition further comprises a chemical mediator. In some embodiments, the chemical mediator is a phenolic compound. In some embodiments, the chemical mediator is a phenolic compound is selected from the group consisting of syringonitrile, acetosyringone, and methyl syringate.

In another aspect, a method of bleaching a surface is provided, comprising contacting the surface with a composition comprising one or more of any of the variant laccase enzymes described herein.

These and other aspects and embodiments of present strains and methods will be apparent from the description, including the accompanying Figures.

### BRIEF DESCRIPTION OF THE DRAWINGS

FIG. 1 provides a schematic illustrating the derivation of the MADE host strain, from the quad-deleted derivative strain.

FIG. 2 provides a schematic of the T. reesei ku80 deletion cassette.

FIG. 3 provides a schematic of the pyr2 deletion cassette used to create the Archy2 strain.

FIG. 4 provides a schematic of the hygR deletion cassette used to create the Archy3 strain.

FIG. 5 is a graph showing laccase activity in filamentous fungi transformed with a vector encoding one of seven different laccase glycosylation variants (mut1 to mut7).

FIG. 6 is a graph showing laccase activity in filamentous fungi transformed with a vector encoding one of five different laccase negative charge variants (S1 to S5).

FIG. 7 is a graph showing laccase activity in filamentous fungi transformed with a vector encoding one of four different laccase positive charge variants (S7 to S10).

FIG. 8 is a list of thirteen position 265 variants.

FIG. 9 is a graph showing laccase activity in filamentous fungi transformed with a vector encoding one of 88 independent laccase variants obtained from a SEL1 library. The left-most line (ABST≈400) is the wild type control.

FIG. 10 a graph showing laccase activity in filamentous fungi transformed with a vector encoding one of six position 265 laccase variants that exhibit increased expression or activity.

FIG. 11 is a graph showing laccase activity in filamentous fungi transformed with a vector encoding one of 88 independent laccase variants obtained from a SEL2 library. The left-most line (ABST≈200) is the wild type control.

FIG. 12 is a graph showing laccase activity in filamentous fungi transformed with a vector encoding one of four different laccase position 287 variants.

FIG. 13 is a list of thirteen position 319 variants.

FIG. 14 is a graph showing laccase activity in filamentous fungi transformed with a vector encoding one of 65 independent laccase variants obtained from a SEL3 library. The left-most line (ABST≈270) is the wild type control.

FIG. 15 is a graph showing laccase activity in filamentous fungi transformed with a vector encoding one of four different laccase position 319 variants.

FIG. 16 is a graph showing laccase activity in filamentous fungi transformed with a vector encoding one of sixteen independent laccase variants obtained from a SEL4 library. The left-most line (ABST≈320) is the wild type control.

FIG. 17 is a graph showing laccase activity in filamentous fungi transformed with a vector encoding one of five different laccase variants.

FIG. 18 is a graph showing laccase activity in filamentous fungi transformed with a vector encoding one of six different laccase variants.

FIG. 19 is a graph showing laccase activity in filamentous fungi transformed with a vector encoding a wt (clones “42”) laccase or an F68L/I265R variant laccase (clones “67”) and grown in shake flasks.

FIG. 20 is an alignment of the amino acid sequences of a number of Cerrena laccases. Signal sequences are shown in italics.

FIG. 21 is an alignment of the amino acid sequences of a number of laccases from different organisms.

FIG. 22 is an amino acid sequence showing the relative location of a number of N-glycosylation mutations (bold), surface charge mutations (bold, underlined), and non-conservative hydrophobic residue mutations (bold, underlined) on a Cerrena laccase D amino acid sequence (SEQ ID NO: 11).

### DETAILED DESCRIPTION

### I. Overview

Described are compositions, methods, and systems, relating to variant laccase enzymes that demonstrate increased expression and/or activity compared to a reference parental laccase enzyme. The variant enzymes include mutations that affect glycosylation, alter the surface charge, alter the surface hydrophobicity, or otherwise alter the biochemical properties of the variant laccase enzymes to improve enzyme expression and/or enzyme activity. Various features and embodiments of the variants laccases, and applications, thereof, are to be described.

### II. Definitions

Unless defined otherwise herein, all technical and scientific terms used herein have the same meaning as commonly understood by one of ordinary skill in the art. Singleton et al., DICTIONARY OF MICROBIOLOGY AND MOLECULAR BIOLOGY, 2D ED., John Wiley and Sons, New York (1994), and Hale and Marham, THE HARPER COLLINS DICTIONARY OF BIOLOGY, Harper Perennial, N.Y. (1991) provide a general dictionary of many of the terms used herein. The following terms are defined for additional clarity.

As used herein, the term “enzyme” refers to a protein that catalyzes a chemical reaction. The catalytic function of an enzyme constitutes its “enzymatic activity” or “activity.” An enzyme is typically classified according to the type of reaction it catalyzes, e.g., oxidation of phenols, hydrolysis of peptide bonds, incorporation of nucleotides, etc.

As used herein, the term “substrate” refers to a substance (e.g., a chemical compound) on which an enzyme performs its catalytic activity to generate a product.

As used herein, a “laccase” is a multi-copper containing oxidase (EC 1.10.3.2) that catalyzes the oxidation of phenols, polyphenols, and anilines by single-electron abstraction, with the concomitant reduction of oxygen to water in a four-electron transfer process.

As used herein, “laccase activity” (or “laccase specific activity”) is measured in units/gram (U/g), wherein one unit is defined as the amount of laccase activity required to oxidize 1 nmol of 2,2′-azinobis(3-ethylbenzthiazoline-6-sulfonate; ABTS) substrate per second under conditions of an assay based on the ability of laccase enzyme to oxidize ABTS into its corresponding stable cation radical, i.e., ABTS⁺. Unlike the initial form of ABTS, the radical form is dark green in color with increased absorbance at 420 nm. The amount of green color formation is proportional to the amount of laccase activity, and can be compared to a laccase standard curve to determine the absolute amount of laccase activity.

As used herein, “expression,” in the context of increased laccase expression, refers to the production of active laccase enzyme molecules in cultured cells.

As used herein, “variant” proteins encompass related and derivative proteins that differ from a parent/reference protein by a small number of amino acid substitutions, insertions, and/or deletions. In some embodiments, the number of different amino acid residues is any of about 1, 2, 3, 4, 5, 10, 20, 25, 30, 35, 40, 45, or 50. In some embodiments, variants differ by about 1 to about 10 amino acids residues. In some embodiments, variant proteins have at least about 35%, 40%, 45%, 50%, 55%, 60%, 65%, 70%, 75%, 80%, 85%, 90%, 91%, 92%, 93%, 94%, 95%, 96%, 97%, 98%, 99%, or even 99.5% amino acid sequence identity to a parent/reference protein.

As used herein, the term “analogous sequence” refers to a polypeptide sequence within a protein that provides a similar function, tertiary structure, and/or conserved residues with respect to a sequence within a parent/reference protein. For example, in structural regions that contain an alpha helix or a beta sheet structure, replacement amino acid residues in an analogous sequence maintain the same structural feature. In some embodiments, analogous sequences result in a variant protein that exhibits a similar or improved function with respect to the parent protein from which the variant is derived.

As used herein, a “homologous protein” or “homolog” refers to a protein (e.g., a laccase enzyme) that has a similar function (e.g., enzymatic activity) and/or structure as a reference protein (e.g., a laccase enzyme from a different source). Homologs may be from evolutionarily related or unrelated species. In some embodiments, a homolog has a quaternary, tertiary and/or primary structure similar to that of a reference protein, thereby potentially allowing for replacement of a segment or fragment in the reference protein with an analogous segment or fragment from the homolog, with reduced disruptiveness of structure and/or function of the reference protein in comparison with replacement of the segment or fragment with a sequence from a non-homologous protein.

As used herein, “wild-type,” “native,” and “naturally-occurring” proteins are those found in nature. The terms “wild-type sequence” refers to an amino acid or nucleic acid sequence that is found in nature or naturally occurring. In some embodiments, a wild-type sequence is the starting point of a protein engineering project, for example, production of variant proteins.

As used herein, a “signal sequence” refers to a sequence of amino acids bound to the N-terminal portion of a protein, and which facilitates the secretion of the mature form of the protein from the cell. The mature form of the extracellular protein lacks the signal sequence which is cleaved off during the secretion process.

As used herein, the term “derivative” refers to a protein that is derived from a parent/reference protein by addition of one or more amino acids to either or both the N- and C-terminal end(s), substitution of one or more amino acid residues at one or a number of different sites in the amino acid sequence, deletion of one or more amino acid residues at either or both ends of the protein or at one or more sites in the amino acid sequence, and/or insertion of one or more amino acids at one or more sites in the amino acid sequence. The preparation of a protein derivative is often achieved by modifying a DNA sequence which encodes for the native protein, transformation of that DNA sequence into a suitable host, and expression of the modified DNA sequence to form the derivative protein.

As used herein, the terms “polypeptide, “protein,” and “peptide,” refer to a composition comprised of amino acids (i.e., amino acid residues). The conventional one-letter or three-letter codes for amino acid residues are used. A polypeptide may be linear or branched, may comprise modified amino acids, and may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “conserved amino acid residue” refers to a residue that is the same at equivalent positions (based on an amino acid sequence alignment) of different laccase enzymes. In contrast, a “non-conserved amino acid residue” refers to a residue that is different at the equivalent positions (based on an amino acid sequence alignment) of different laccase enzymes. By way of example, where numerous laccases have an alanine at position X, alanine is a conserved residue at position X. Where different laccases have different amino acids at position Y, there are a number of non-conserved residues at position Y.

As used herein, “equivalent” amino acid positions/residues are those that are structurally conserved among different laccase enzymes as determined by an amino acid sequence alignment. Such positions/residues can readily be determined using any one of a number of amino acid sequence alignment programs, and then determining which positions/residues “line-up” in different molecules. The language “equivalent to” and “corresponding to” are used interchangeably.

As used herein, the term “textile” refers to fibers, yarns, fabrics, garments, and non-woven materials. The term encompasses textiles made from natural and synthetic (e.g., manufactured) materials, as well as natural and synthetic blends. The term “textile” refers to both unprocessed and processed fibers, yarns, woven or knit fabrics, non-wovens, and garments. In some embodiments, a textile contains cellulose.

As used herein, the term “fabric” refers to a manufactured assembly of fibers and/or yarns that has substantial surface area in relation to its thickness and sufficient cohesion to give the assembly useful mechanical strength.

As used herein, the term “garment” refers to a clothing item made from one or more fabrics. Garments typically include fabrics that are already cut to size and sewn or stitched together. Garments may or may not include buttons, eyelets, straps, zippers, hook-and-loop closures, or other mechanical features, which can be attached before or after localized color modification.

As used herein, the term “color modification” refers to a change in the chroma, saturation, intensity, luminance, and/or tint of a color associated with a fiber, yarn, fabric, garment, or non-woven material, collectively referred to as textile materials. Color modification encompasses chemical modification to a chromophore as well as chemical modification to the material to which a chromophore is attached. Examples of color modification include fading, bleaching, and altering tint. A particular color modification to indigo-dyed denim is fading to a “vintage look,” which has a less intense blue/violet tint and more subdued grey appearance than the freshly-dyed denim.

As used herein, the term “local color modification” refers to color modification, as defined, above, that is performed on only a portion of a fabric or garment. Unlike generalized textile color modification, which is typically performed in a bath, i.e., in a submerged environment, local color modification is performed using a wetted but not submerged fabric or garment, typically on a table, work bench, or other hard surface, on a hanging or otherwise suspended fabric or garment, or using rollers or other processing equipment that do not subject the fabric or garment to a submerged environment, such that only a portion of the garment can be subjected to color modification without affecting the remainder of the fabric or garment.

As used herein, “a portion of a fabric or garment” refers to anything less than the whole fabric or garment. Where specified, a portion of a fabric or garment may refer to an indicated structural or decorative feature a fabric or garment, such as a pant leg, a sleeve, a pocket, a belt loop, a cuff, a hem, and the like.

As used herein, the term “bleaching” refers to the process of treating a textile material such as a fiber, yarn, fabric, garment or non-woven material to produce a lighter color. This term includes the production of a brighter and/or whiter textile, e.g., in the context of a textile processing application, as well as lightening of the color of a stain, e.g., in the context of a cleaning application.

As used herein, the terms “size” and “sizing” refer to compounds used in the textile industry to improve weaving performance by increasing the abrasion resistance and strength of a yarn. Size is usually made of starch or starch-like compounds.

As used herein, the terms “desize” and “desizing” refer to the process of eliminating/removing size (generally starch) from a textile, usually prior to applying special finishes, dyes or bleaches.

As used herein, the term “desizing enzyme” refers to an enzyme used to remove size. Exemplary enzymes are amylases, cellulases, and mannanases.

As used herein, the term “% identity” refers to the level of nucleic acid sequence identity between a nucleic acid sequence that encodes a laccase as described herein and another nucleic acid sequence, or the level of amino acid sequence identity between a laccase enzyme as described herein and another amino acid sequence. Alignments may be performed using a conventional sequence alignment program. Exemplary levels of nucleic acid and amino acid sequence identity include, but are not limited to, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, or more, sequence identity to a given sequence, e.g., the coding sequence for a laccase or the amino acid sequence of a laccase, as described herein.

Exemplary computer programs that can be used to determine identity between two sequences include, but are not limited to, the suite of BLAST programs, e.g., BLASTN, BLASTX, and TBLASTX, BLASTP and TBLASTN, publicly available on the Internet at www.ncbi.nlm.nih.gov/BLAST. See also, Altschul, et al., 1990 and Altschul, et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, et al., 1997.)

An alignment of selected sequences in order to determine “% identity” between two or more sequences, may be performed using, for example, the CLUSTAL-W program in MacVector version 6.5, operated with default parameters, including an open gap penalty of 10.0, an extended gap penalty of 0.1, and a BLOSUM 30 similarity matrix.

As used herein, the terms “chemical mediator” and “mediator” are used interchangeably to refer to a chemical compound that functions as a redox mediator to shuttle electrons between an enzyme exhibiting oxidase activity (e.g., a laccase) and a secondary substrate or electron donor. Such chemical mediators are also known in the art as “enhancers” and “accelerators.”

As used herein, the terms “secondary substrate” and “electron donor” are used interchangeably to refer to a dye, pigment (e.g., indigo), chromophore (e.g., polyphenolic, anthocyanin, or carotenoid), or other secondary substrate to and from which electrons can be shuttled by an enzyme exhibiting oxidase activity.

The following abbreviations/acronyms have the following meanings unless otherwise specified:

EC enzyme commission

EDTA ethylenediaminetetraacetic acid

kDa kiloDalton

MW molecular weight

w/v weight/volume

w/w weight/weight

v/v volume/volume

wt % weight percent

° C. degrees Centigrade

H₂O water

dH₂O or DI deionized water

dIH₂O deionized water, Milli-Q filtration

g or gm gram

μg microgram

mg milligram

kg kilogram

μL and μl microliter

mL and ml milliliter

mm millimeter

μm micrometer

M molar

mM millimolar

μM micromolar

U unit

sec and ″ second

min and ′ minute

hr hour

eq. equivalent

N normal

RTU ready-to-use

U Unit

owg on weight of goods

CIE International Commission on Illumination

Numeric ranges are inclusive of the numbers defining the range. The singular articles “a,” “an,” “the,” and the like, include the plural referents unless otherwise clear from context. Unless otherwise specified, polypeptides are written in the standard N-terminal to C-terminal direction and polynucleotides are written in the standard 5′ to 3′ direction. It is to be understood that the particular methodologies, protocols, and reagents described, are not intended to be limiting, as equivalent methods and materials can be used in the practice or testing of the present compositions and methods. Although the description is divided into sections to assist the reader, section heading should not be construed as limiting and the description in one section may apply to another. All publications cited herein are expressly incorporated by reference.

III. Parental laccases

A number of laccase enzymes from microbial and plant origin are known in the art. Exemplary laccases are derived or derivable from a strain of Aspergillus, Neurospora (e.g., N. crassa), Podospora, Botrytis, Collybia, Cerrena (e.g., C. unicolor), Stachybotrys, Panus (e.g., P. rudis), Thielavia, Fomes, Lentinus, Pleurotus, Trametes (e.g., T. villosa, and T. versicolor), Rhizoctonia (e.g., R. solani), Coprinus (e.g., C. plicatilis and C. cinereus), Psatyrella, Myceliophthora (e.g., M. thermonhila), Schytalidium, Phlebia (e.g., P. radita (WO 92/01046)), or Coriolus (e.g., C. hirsutus (JP 2238885)), Spongipellis, Polyporus, Ceriporiopsis subvermispora, Ganoderma tsunodae, and Trichoderma.

The following laccases are described in U.S. Patent Publication No. 2008/0196173 and PCT Publication No. WO 2008/076322 (which are incorporated by reference) and are ideal for use as described:

| A. Cerrena laccase A1 from CBS115.075 strain (SEQ ID NO: 1): | |
|---|---|
| MSSKLLALIT VALVLPLGTD AGIGPVTDLR ITNQDIAPDG FTRPAVLAGG | 50 |
| TFPGALITGQ KGDSFQINVI DELTDASMLT QTSIHWHGFF QKGSAWADGP | 100 |
| AFVTQCPIVT GNSFLYDFDV PDQPGTFWYH SHLSTQYCDG LRGPFVVYDP | 150 |
| KDPNKRLYDI DNDHTVITLA DWYHVLARTV VGVATPDATL INGLGRSPDG | 200 |
| PADAELAVIN VKRGKRYRFR LVSISCDPNY IFSIDNHSMT VIEVDGVNTQ | 250 |
| SLTVDSIQIF AGQRYSFVLH ANRPENNYWI RAKPNIGTDT TTDSGMNSAI | 300 |
| LRYNGAPVAE PQTVQSPSLT PLLEQNLRPL VYTPVPGNPT PGGADIVHTL | 350 |
| DLSFDAGRFS INGASFLDPT VPVLLQILSG TQNAQDLLPP GSVIPLELGK | 400 |
| VVELVIPAGV VGGPHPFHLH GHNFWVVRSA GTDQYNFNDA ILRDVVSIGG | 450 |
| TGDQVTIRFV TDNPGPWFLH CHIDWHLEAG LAIVFAEGIE NTAASNLTPQ | 500 |
| AWDELCPKYN ALSAQKKLNPSTT | 523 |
| B. Cerrena laccase A2 from CBS154.29 strain (SEQ ID NO: 2): | |
| MSSKLLALIT VALVLPLGTD AGIGPVTDLR ITNQDIAPDG FTRPAVLAGG | 50 |
| TFPGALITGQ KGDSFQINVI DELTDASMLT QTSIHWHGFF QKGSAWADGP | 100 |
| AFVTQCPIVT GNSFLYDFDV PDQPGTFWYH SHLSTQYCDG LRGPFVVYDP | 150 |
| KDPNKRLYDI DNDHTVITLA DWYHVLARTV VGVATPDATL INGLGRSPDG | 200 |
| PADAELAVIN VKRGKRYRFR LVSISCDPNY IFSIDNHSMT VIEVDGVNTQ | 250 |
| SLTVDSIQIF AGQRYSFVLH ANRPENNYWI RAKPNIGTDT TTDNGMNSAI | 300 |
| LRYNGAPVAE PQTVQSPSLT PLLEQNLRPL VYTPVPGNPT PGGADIVHTL | 350 |
| DLSFDAGRFS INGASFLDPT VPVLLQILSG TQNAQDLLPP GSVIPLELGK | 400 |
| VVELVIPAGV VGGPHPFHLH GHNFWVVRSA GTDQYNFNDA ILRDVVSIGG | 450 |
| TEDQVTIRFV TDNPGPWFLH CHIDWHLEAG LAIVFAEGIE NTAASNPTPQ | 500 |
| AWDELCPKYN ALNAQKKLNP STT | 523 |
| C. Cerrena laccase B1 from CBS115.075 strain (SEQ ID NO: 3): | |
| MSLLRSLTSL IVLVIGAFAA IGPVTDLHIV NQNLDPDGFN RPTVLAGGTF | 50 |
| PGPLIRGNKG DNFKINVIDD LTEHSMLKAT SIHWHGFFQK GTNWADGPAF | 100 |
| VTQCPITSGN AFLYDFNVPD QAGTFWYHSH LSTQYCDGLR GAFVVYDPND | 150 |
| PNKQLYDVDN GNTVITLADW YHALAQTVTG VAVSDATLIN GLGRSATGPA | 200 |
| NAPLAVISVE RNKRYRFRLV SISCDPNFIF SIDHHPMTVI EMDGVNTQSM | 250 |
| TVDSIQIFAG QRYSFVMQAN QPVGNYWIRA KPNVGNTTFL GGLNSAILRY | 300 |
| VGAPDQEPTT DQTPNSTPLV EANLRPLVYT PVPGQPFPGG ADIVKNLALG | 350 |
| FNAGRFTING ASLTPPTVPV LLQILSGTHN AQDLLPAGSV IELEQNKVVE | 400 |
| IVLPAAGAVG GPHPFHLHGH NFWVVRSAGQ TTYNFNDAPI RDVVSIGGAN | 450 |
| DQVTIRFVTD NPGPWFLHCH IDWHLEAGFA VVFAEGINGT AAANPVPAAW | 500 |
| NQLCPLYDAL SPGDT | 515 |
| D. Cerrena laccase B2 from CBS154.29 strain (SEQ ID NO: 4): | |
| MSLLRSLTSL IVLATGAFAA IGPVTDLHIV NQNLAPDGLN RPTVLAGGTF | 50 |
| PGPLIRGNKG DNFKINVIDD LTEHSMLKAT SIHWHGFFQK GTNWADGPAF | 100 |
| VTQCPITSGN AFLYDFNVPD QAGTFWYHSH LSTQYCDGLR GAFVVYDPND | 150 |
| PNKQLYDVDN GNTVITLADW YHALAQTVTG VAVSDATLIN GLGRSATGPA | 200 |
| NAPLAVISVE RNKRYRFRLV SISCDPNFIF SIDHHPMTVI EMDGVNTQSM | 250 |
| TVDSIQIFAG QRYSFVMQAN QPVGNYWIRA KPNVGNTTFL GGLNSAILRY | 300 |
| VGAPDQEPTT DQTPNSTPLV EANLRPLVYT PVPGQPFPGG ADIVKNLALG | 350 |
| FNAGRFTING TSFTPPTVPV LLQILSGTHN AQDLLPAGSV IELEQNKVVE | 400 |
| IVLPAAGAVG GPHPFHLHGH NFWVVRSAGQ TTYNFNDAPI RDVVSIGGAN | 450 |
| DQVTIRFVTD NPGPWFLHCH IDWHLEAGFA VVFAEGINGT AAANPVPAAW | 500 |
| NQLCPLYDAL SPGDT | 515 |
| E. Cerrena laccase B3 (partial) from ATCC20013 strain (SEQ | |
| ID NO: 5): | |
| MSLLRSLTSL IVLATGAFAA IGPVTDLHIV NQNLAPDGFN RPTVLAGGTF | 50 |
| PGPLIRGNKG DNFKINVIDD LTEHSMLKAT SIHWHGFFQK GTNWADGPAF | 100 |
| VTQCPITSGN SFLYDFNVPD QAGTFWYHSH LSTQYCDGLR GAFVVYDPND | 150 |
| PNKQLYDVDN GKTVITLADW YHALAQTVTG VAVSDATLIN GLGRSATGPA | 200 |
| NAPLAVISVE RNKRYRFRLV SISCDPNFIF SIDHHPMTVI EMDGVNTQSM | 250 |
| TVDSIQIFAG QRYSFVMQAN QPVGNYWI | 278 |
| F. Cerrena laccase C (partial) from CBS154.29 strain (SEQ | |
| ID NO: 6): | |
| AIGPVADLHI TDDTIAPDGF SRPAVLAGGG FPGPLITGNK GDAFKLNVID | 50 |
| ELTDASMLKX TSIHWHGFFQ KGTNWADGPA FVNQCPITTG NSFLYDFQVP | 100 |
| DQAGTYWYHS HLSTQYCDGL RGAFVVYDPS DPHKDLYDVD DESTVITLAD | 150 |
| WYHTLARQIV GVAISDTTLI NGLGRNTNGP ADAALAVINV DAGKRYRFRL | 200 |
| VSISCDPNWV FSIDNHDFTV IEVDGVNSQP LNVDSVQIFA GQRYSF | 246 |
| G. Cerrena laccase D1 from CBS154.29 strain (SEQ ID NO: 7): | |
| MGLNSAITSL AILALSVGSY AAIGPVADIH IVNKDLAPDG VQRPTVLAGG | 50 |
| TFPGTLITGQ KGDNFQLNVI DDLTDDRMLT PTSIHWHGFF QKGTAWADGP | 100 |
| AFVTQCPIIA DNSFLYDFDV PDQAGTFWYH SHLSTQYCDG LRGAFVVYDP | 150 |
| NDPHKDLYDV DDGGTVITLA DWYHVLAQTV VGAATPDSTL INGLGRSQTG | 200 |
| PADAELAVIS VEHNKRYRFR LVSISCDPNF TFSVDGHNMT VIEVDGVNTR | 250 |
| PLTVDSIQIF AGQRYSFVLN ANQPEDNYWI RAMPNIGRNT TTLDGKNAAI | 300 |
| LRYKNASVEE PKTVGGPAQS PLNEADLRPL VPAPVPGNAV PGGADINHRL | 350 |
| NLTFSNGLFS INNASFTNPS VPALLQILSG AQNAQDLLPT GSYIGLELGK | 400 |
| VVELVIPPLA VGGPHPFHLH GHNFWVVRSA GSDEYNFDDA ILRDVVSIGA | 450 |
| GTDEVTIRFV TDNPGPWFLH CHIDWHLEAG LAIVFAEGIN QTAAANPTPQ | 500 |
| AWDELCPKYN GLSASQKVKP KKGTAI | 526 |
| H. Cerrena laccase D2 from CBS115.075 strain (SEQ ID NO: 8): | |
| MGLNSAITSL AILALSVGSY AAIGPVADIH IVNKDLAPDG VQRPTVLAGG | 50 |
| TFPGTLITGQ KGDNFQLNVI DDLTDDRMLT PTSIHWHGFF QKGTAWADGP | 100 |
| AFVTQCPIIA DNSFLYDFDV PDQAGTFWYH SHLSTQYCDG LRGAFVVYDP | 150 |
| NDPHKDLYDV DDGGTVITLA DWYHVLAQTV VGAATPDSTL INGLGRSQTG | 200 |
| PADAELAVIS VEHNKRYRFR LVSISCDPNF TFSVDGHNMT VIEVDGVNTR | 250 |
| PLTVDSIQIF AGQRYSFVLN ANQPDDNYWI RAMPNIGRNT TTLDGKNAAI | 300 |
| LRYKNASVEE PKTVGGPAQS PLNEADLRPL VPAPVPGNAV PGGADINHRL | 350 |
| NLTFSNGLFS INNASFTNPS VPALLQILSG AQNAQDLLPT GSYIGLELGK | 400 |
| VVELVIPPLA VGGPHPFHLH GHNFWVVRSA GSDEYNFDDA ILRDVVSIGA | 450 |
| GTDEVTIRFV TDNPGPWFLH CHIDWHLEAG LAIVFAEGIN QTAAANPTPQ | 500 |
| AWDELCPKYN GLSASQKVKP KKGTAI | 526 |
| I. Cerrena laccase E (partial) from CBS154.29 strain (SEQ | |
| ID NO: 9): | |
| AIGPVADLKI VNRDIAPDGF IRPAVLAGGS FPGPLITGQK GNEFKINVVN | 50 |
| QLTDGSMLKS TSIHWHGFFQ KGTNWADGPA FVNQCPIATN NSFLYQFTSQ | 100 |
| EQPGTFWYHS HLSTQYCDGL RGPLVVYDPQ DPHAVLYDVD DESTIITLAD | 150 |
| WYHTLARQVK GPAVPGTTLI NGLGRHNNGP LDAELAVISV QAGKRQVQFT | 200 |
| LFTLYRFRLI SISCDPNYVF SIDGHDMTVI EVDSVNSQPL KVDSIQIFAG | 250 |
| QRYSFVLNAN QP | 262 |
| In some embodiments, a laccase D enzyme having the following | |
| amino acid sequence (SEQ ID NO: 10; signal sequence in | |
| italics) may be used: | |
| MGLNSAITSL AILALSVGSY AAIGPVADLH IVNKDLAPDG VQRPTVLAGG | 50 |
| TFPGTLITGQ KGDNFQLNVI DDLTDDRMLT PTSIHWHGFF QKGTAWADGP | 100 |
| AFVTQCPIIA DNSFLYDFDV PDQAGTFWYH SHLSTQYCDG LRGAFVVYDP | 150 |
| NDPHKDLYDV DDGGTVITLA DWYHVLAQTV VGAATPDSTL INGLGRSQTG | 200 |
| PADAELAVIS VEHNKRYRFR LVSISCDPNF TFSVDGHNMT VIEVDGVNTR | 250 |
| PLTVDSIQIF AGQRYSFVLN ANQPEDNYWI RAMPNIGRNT TTLDGKNAAI | 300 |
| LRYKNASVEE PKTVGGPAQS PLNEADLRPL VPAPVPGNAV PGGADINHRL | 350 |
| NLTFSNGLFS INNASFTNPS VPALLQILSG AQNAQDLLPT GSYIGLELGK | 400 |
| VVELVIPPLA VGGPHPFHLH GHNFWVVRSA GSDEYNFDDA ILRDVVSIGA | 450 |
| GTDEVTIRFV TDNPGPWFLH CHIDWHLEAG LAIVFAEGIN QTAAANPTPQ | 500 |
| AWDELCPKYN GLSASQKVKP KKGTAI | 526 |

The mature processed form of this polypeptide is as follows
(SEQ ID NO: 11):
AIGPVADLHIVNKDLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI

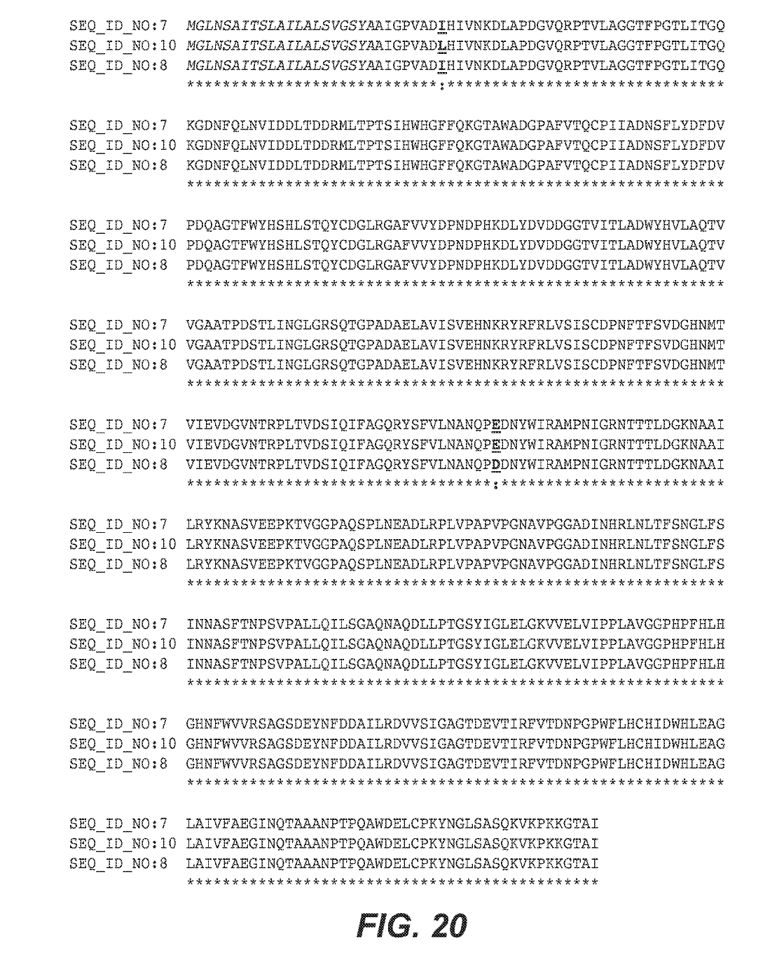

Note that SEQ ID NO: 7 (Cerrena laccase D1), SEQ ID NO: 8 (Cerrena laccase D2), and SEQ ID NO: 10 (Cerrena laccase D) are nearly identical, except for position 8 (where SEQ ID NO: 7 and SEQ ID NO: 8 have Ile and SEQ ID NO: 10 has Leu) and position 254 (where SEQ ID NO: 7 and SEQ ID NO: 10 have Glu and SEQ ID NO: 8 has Asp), using the mature form of the laccase of SEQ ID NO: 10 (i.e., SEQ ID NO: 11) for numbering (see, e.g., FIG. 20). These differences do not appear to substantially affect laccase expression or specific activity.

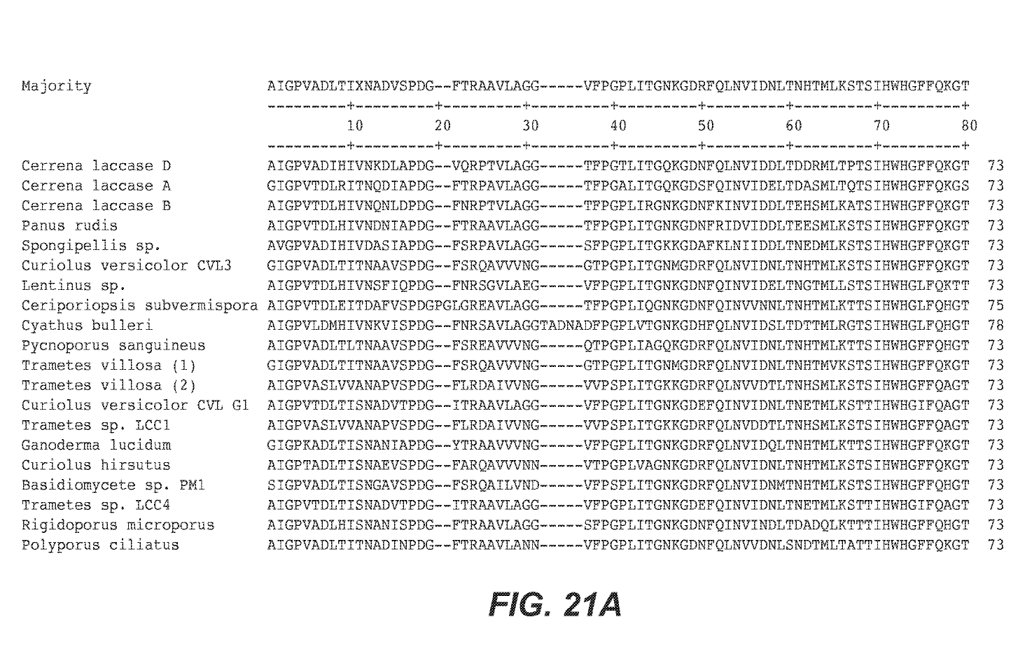

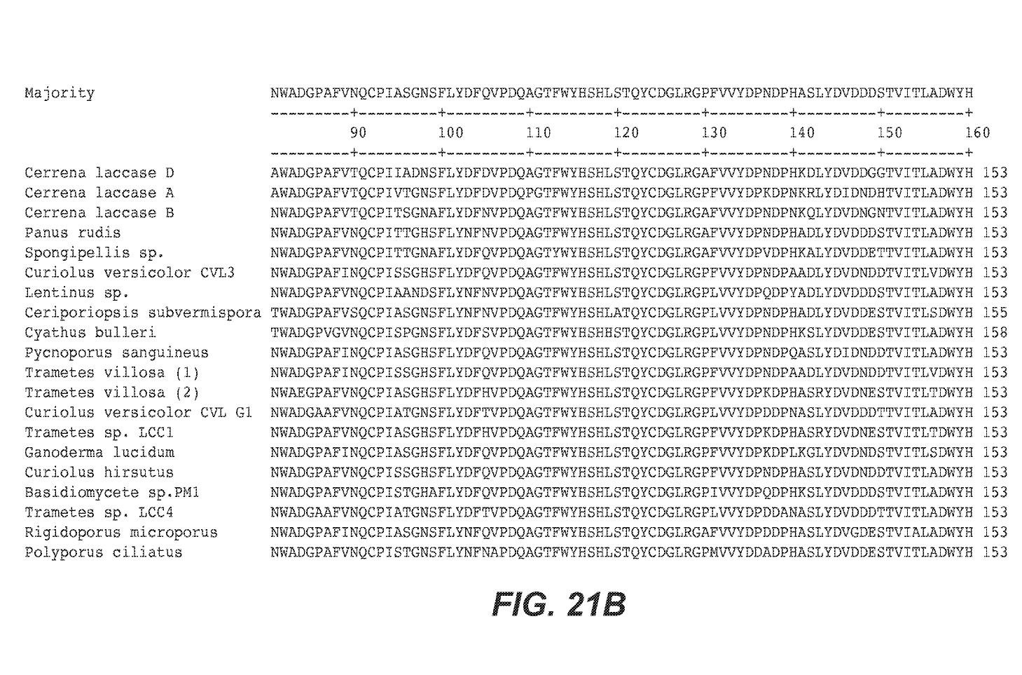

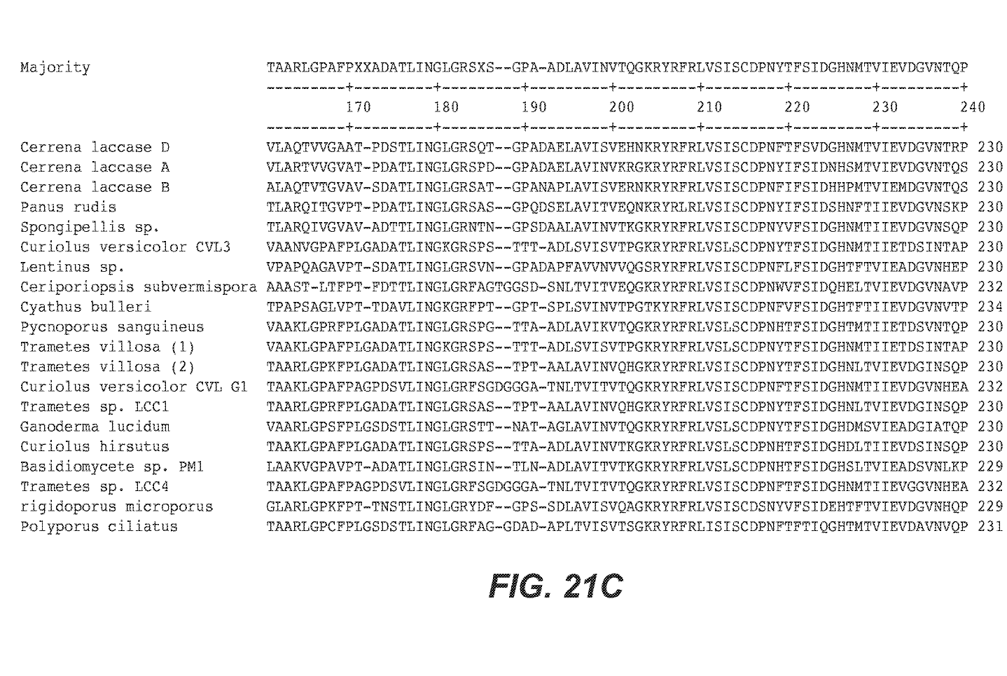

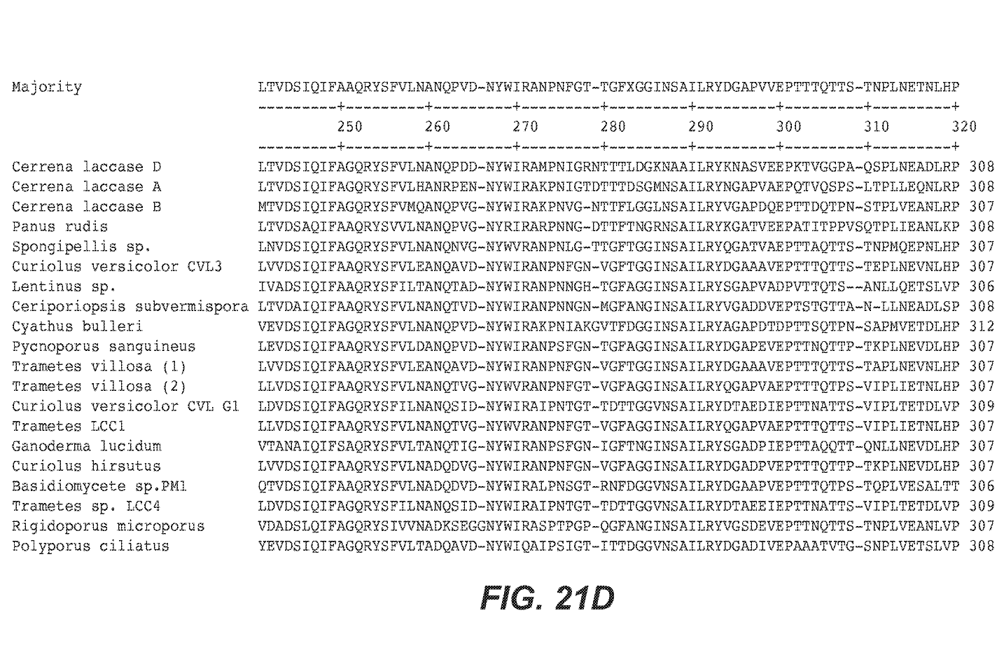

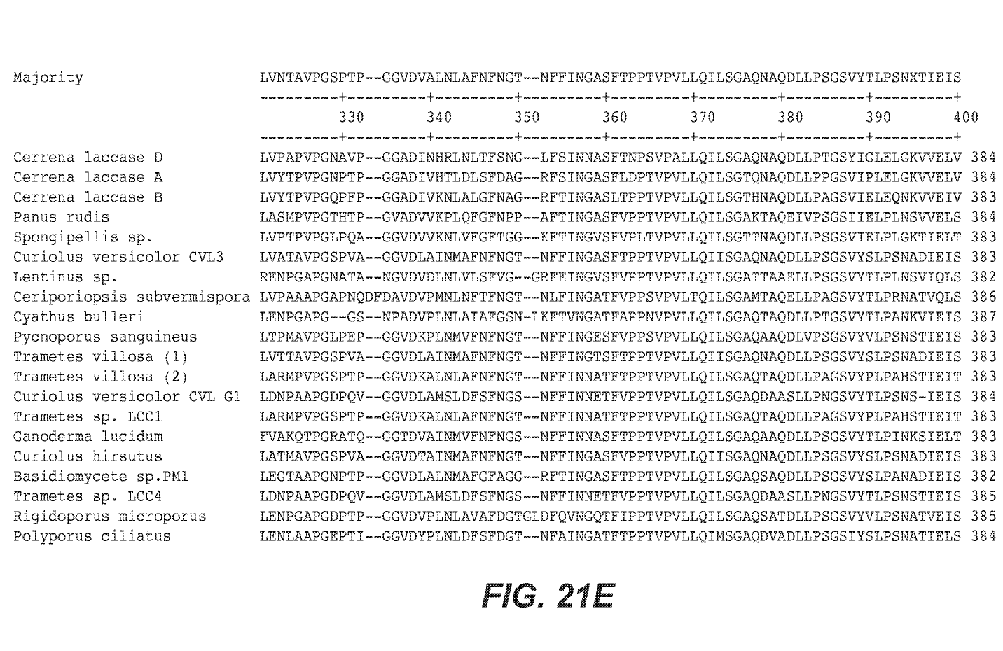

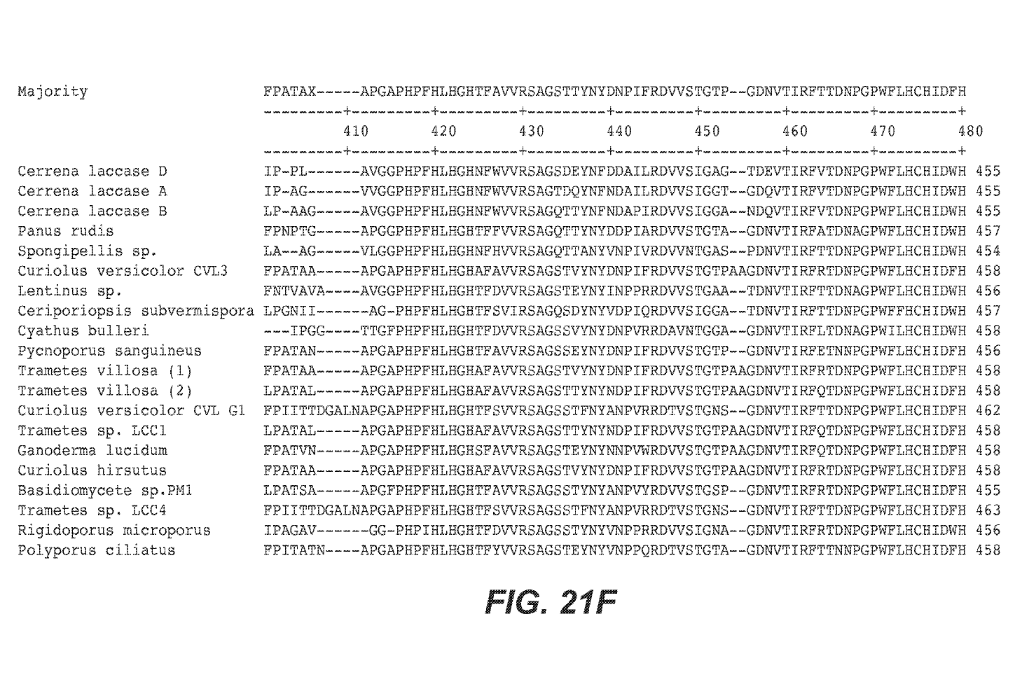

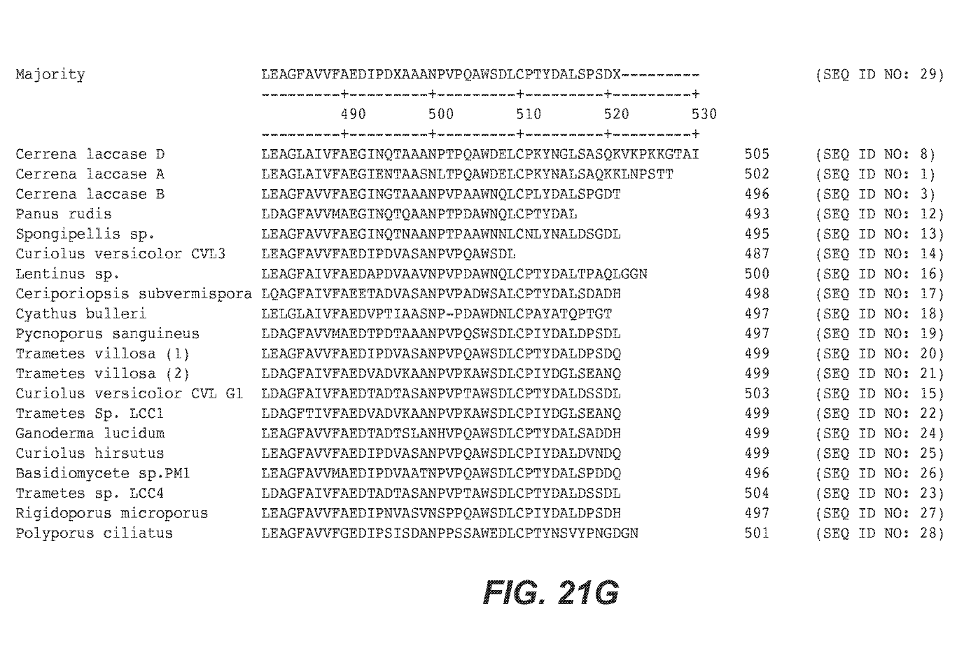

Additional laccases include but are not limited to those shown in the alignment in FIG. 21, i.e., Panus rudis (SEQ ID NO: 12), Spongipellis sp. (SEQ ID NO: 13), Curiolus versicolor CVL3 (SEQ ID NO: 14), Curiolus versicolor CVL G1 (SEQ ID NO: 15), Lentinus sp. (SEQ ID NO: 16), Ceriporiopsis subvermispora (SEQ ID NO: 17), Cyathus bulleri (SEQ ID NO: 18), Pycnoporus sanguineus (SEQ ID NO: 19), Trametes villosa (1) and (2) (SEQ ID NOs: 20 and 21, respectively), Trametes sp. LCC1 (SEQ ID NO: 22), Trametes sp. LCC4 (SEQ ID NO: 23), Ganodenna lucidum (SEQ ID NO: 24), Curiolus hirsutus (SEQ ID NO: 25), Basidiomycete sp. PM1 (SEQ ID NO: 26), Rigidoporus microporus (SEQ ID NO: 27), and Polyporus ciliatus (SEQ ID NO: 28). A consensus (or majority) amino acid sequence is shown as SEQ ID NO: 29.

A laccase may be produced by culturing a host cell transformed with a recombinant DNA vector that includes nucleotide sequences encoding the laccase. The DNA vector may further include nucleotide sequences permitting the expression of the laccase in a culture medium, and optionally allowing the recovery of the laccase from the culture.

An expression vector containing a polynucleotide sequence encoding a laccase enzyme may be transformed into a suitable host cell. The host cell may be a fungal cell, such as a filamentous fungal cell, examples of which include but are not limited to species of Trichoderma [e.g., T. reesei (previously classified as T. longibrachiatum and currently also known as Hypocrea ecorina], T. viride, T. koningii, and T. harzianum), Aspergillus (e.g., A. niger, A. nidulans, A. oryzae, and A. awamori), Penicillium, Humicola (e.g., H. insolens and H. grisea), Fusarium (e.g., F. graminum and F. venenatum), Neurospora, Hypocrea, and Mucor. A host cell for expression of a laccase enzyme may also be from a species of Cerrena (e.g., C. unicolor). Fungal cells may be transformed by a process involving protoplast formation and transformation of the protoplasts followed by regeneration of the cell wall using techniques known in the art.

Alternatively, the host organism may from a species of bacterium, such as Bacillus [e.g., B. subtilis, B. lichenifonnis, B. lentus, B. (now Geobacillus) stearothermophilus, and B. brevis], Pseudomonas, Streptomyces (e.g., S. coelicolor, S. lividans), or E. coli. The transformation of bacterial cells may be performed according to conventional methods, e.g., as described in Maniatis, T. et al., “Molecular Cloning: A Laboratory Manual,” Cold Spring Harbor, 1982. The screening of appropriate DNA sequences and construction of vectors may also be carried out by standard procedures (cf. supra).

The medium used to culture the transformed host cells may be any conventional medium suitable for growing the host cells. In some embodiments, the expressed enzyme is secreted into the culture medium and may be recovered therefrom by well-known procedures. For example, laccases may be recovered from a culture medium as described in U.S. Patent Publication No. 2008/0196173. In some embodiments, the enzyme is expressed intracellularly and is recovered following disruption of the cell membrane.

In particular embodiments, the expression host may be Trichoderma reesei with the laccase coding region under the control of a CBH1 promoter and terminator (see, e.g., U.S. Pat. No. 5,861,271). The expression vector may be, e.g., pTrex3g, as disclosed in U.S. Pat. No. 7,413,887. In some embodiments, laccases are expressed as described in U.S. Patent Publication Nos. 2008/0196173 or 2009/0221030.

In some embodiments, laccase enzymes suitable for use in the present compositions and methods are mature polypeptides that lack a signal sequence that may be used to direct secretion of a full-length polypeptide from a cell.

A suitable mature polypeptide may have at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, or more, amino acid sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28. Preferably, such polypeptides have enzymatic laccase activity, as determined using the assays and procedures described, herein.

In some embodiments, laccase enzymes suitable for use in the present compositions and methods are truncated with respect to a full-length or mature parent/reference sequence. Such truncated polypeptides may be generated by the proteolytic degradation of a full-length or mature polypeptide sequence or by engineering a polynucleotide to encode a truncated polypeptide. Exemplary polypeptides are truncated at the amino and/or carboxyl-terminus with respect to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28. The truncation may be of a small number, e.g., 1, 2, 3, 4, 5, 6, 7, 8, 9, or 10 amino acid residues, or of entire structural or functional domains. A suitable truncated polypeptide may have at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, or more, amino acid sequence identity to the corresponding portion of one or more of the above-references amino acid sequences.

Preferably, such polypeptides have enzymatic laccase activity, as determined using the assays and procedures described, herein.

### IV. Variant Laccases

The present compositions, methods, and systems feature a variant laccase demonstrating increased expression and/or specific activity compared to the parental laccase from which it is derived, for example, the parental laccase of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the variant laccase include mutations that introduce a glycosylation site into the laccase amino acid sequence. The glycosylation site is preferrably on the surface of the laccase enzyme. In some embodiments, the mutation introduces an N-glycosylation site [i.e., the amino acid sequence Asn-Xaa-Thr/Ser (N-X-T/S), where X is any amino acid residue except proline] on the surface of the laccase enzyme. N-glycosylation sites may be introduced into an amino acid sequence by introducing an Asn residue in the correct context, by changing the context of an existing Asn residue, or both. In some cases, a single amino acid mutation is sufficient to introduce an N-glycosylation site. In other cases, two (or even three) amino acid mutations are required to introduce an N-glycosylation site. In some embodiments, an N-glycosylation site is introduced at one, two, three, or more positions equivalent to positions 12, 28, 47, 157, 317, 362, and 492 of the laccase amino acid sequence exemplified by SEQ ID NO: 11.

In particular embodiments, an N-glycosylation site is introduced at a position equivalent to position 12 of SEQ ID NO: 11, e.g., by changing the amino acid sequence NKD to NAT (or NAS, N-V/L/I-T/S) at positions equivalent to positions 12 to 14. In particular embodiments, an N-glycosylation site is introduced at a position equivalent to position 28 of SEQ ID NO: 11, e.g., by changing the amino acid sequence GGT to NGT at positions equivalent to positions 28 to 30. In particular embodiments, an N-glycosylation site is introduced at a position equivalent to position 47 of SEQ ID NO: 11, e.g., by changing the amino acid sequence NVI to NVT (or NVS) at positions equivalent to positions 47 to 49. In particular embodiments, an N-glycosylation site is introduced at a position equivalent to position 157 of SEQ ID NO: 11, e.g., by changing the amino acid sequence QTV to NTT (or NTS) at positions equivalent to positions 157 to 159. In particular embodiments, an N-glycosylation site is introduced at a position equivalent to position 317 of SEQ ID NO: 11, e.g., by changing the amino acid sequence NAV to NAT (or NAS) at positions equivalent to positions 317 to 319. In particular embodiments, an N-glycosylation site is introduced at a position equivalent to position 362 of SEQ ID NO: 11, e.g., by changing the amino acid sequence NAQ to NAS (or NAT) at residues 362 to 364. In particular embodiments, an N-glycosylation site is introduced at a position equivalent to position 492 of SEQ ID NO: 11, e.g., by changing the amino acid sequence SAS to NAS at positions equivalent to positions 492 to 494. Ser and Thr are substitutable in the glycosylation site and are unlikely to affect laccase structure or function. Conservative substitutions in the “X” position are generally also substitutable.

The amino acid sequences of exemplary glycosylation variants derived from C. unicolor laccase D (SEQ ID NO: 11) are shown, below.

NKD to NAT at residues 12 to 14 (G*12, variant mut1; SEQ ID NO: 30):
AIGPVADLHIVNATLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
GGT to NGT at residues 28 to 30 (G*28; variant mut2; SEQ ID NO: 31):
AIGPVADLHIVNKDLAPDGVQRPTVLANGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
NVI to NVT at residues 47 to 49 (G*47; variant mut3; SEQ ID NO: 32):
AIGPVADLHIVNKDLAPDGVQRPTVLAGGTFPGTLITGQKGDNFQLNVTDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
QTV to NTT at residues 157 to 159 (N*157; variant mut4; SEQ ID NO: 33):
AIGPVADLHIVNKDLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLANTTVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
NAV to NAT at residues 317 to 319 (N*317; variant mut5; SEQ ID NO: 34):
AIGPVADLHIVNKDLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNATPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
NAQ to NAS at residues 362 to 364 (N*362; variant mut6; SEQ ID NO: 35):
AIGPVADLHIVNKDLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNASDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLSASQKVKPKKGTAI
SAS to NAS at residues 492 to 494 (N*492; variant mut7; SEQ ID NO: 36):
AIGPVADLHIVNKDLAPDGVQRPTVLAGGTFPGTLITGQKGDNFQLNVIDDLTDDRMLTP
TSIHWHGFFQKGTAWADGPAFVTQCPIIADNSFLYDFDVPDQAGTFWYHSHLSTQYCDGL
RGAFVVYDPNDPHKDLYDVDDGGTVITLADWYHVLAQTVVGAATPDSTLINGLGRSQTGP
ADAELAVISVEHNKRYRFRLVSISCDPNFTFSVDGHNMTVIEVDGVNTRPLTVDSIQIFA
GQRYSFVLNANQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSVPALLQILSGA
QNAQDLLPTGSYIGLELGKVVELVIPPLAVGGPHPFHLHGHNFWVVRSAGSDEYNFDDAI
LRDVVSIGAGTDEVTIRFVTDNPGPWFLHCHIDWHLEAGLAIVFAEGINQTAAANPTPQA
WDELCPKYNGLNASQKVKPKKGTAI

In some embodiments, the variant laccase include mutations that alter the surface charge of the laccase enzyme. In some embodiments, the variant includes a mutation at an amino acid position corresponding to position 130 in SEQ ID NO: 11, wherein the residue in the parental laccase is substituted with a different residue. In some embodiments, the mutation introduces a negative charge at this position. The amino acid in the parental laccase may be N, which as shown in FIGS. 20 and 21 is the consensus amino acid residue at this position, based on an alignment of a number of laccases, but may be a different residue, such as K, Q, D, V, or A. In some embodiments, the substituting residue is D, E, R, or K. In some embodiments, the substituting residue is D or E. In particular embodiments, the mutation corresponds to N130E. In some embodiments, the variant includes a mutation at an amino acid position corresponding to position 335 in SEQ ID NO: 11, wherein the residue in the parental laccase is substituted with a different residue. In some embodiments, the mutation introduces a negative charge at this position. The amino acid in the parental laccase may be N, or a different residue, such as A, P, S, or G. In particular embodiments, the substituting residue is D, E, R, or K. In particular embodiments, the substituting residue is R or K. In particular embodiments, the mutation corresponds to N335R.

In some embodiments, the variant laccase includes a mutation at an amino acid position corresponding to a non-conservative, hydrophobic amino acid residue located on the surface of Cerrena laccase. In some embodiments, the variant includes a mutation at an amino acid position corresponding to position 265, 287, 293, or 319, in SEQ ID NO: 11.

In some embodiments, the variant includes a mutation at an amino acid position corresponding to position 265 in SEQ ID NO: 11, wherein the residue in the parental laccase is substituted with a different residue. In some embodiments, the amino acid in the parental laccase is a small aliphatic amino acid residue. In some embodiments, the amino acid in an aromatic amino acid residue. In some embodiments, the amino acid in the parental laccase is L, V, F, T, N, S, or P. In some embodiments, the amino acid in the parental laccase is I. In some embodiments, the substituting residue is R, H, V, K, I, or L. In particular embodiments, the mutation corresponds to I265R/H/V/K/I/L. In particular embodiments, the mutation corresponds to I265R, I265H, or I265V.

In some embodiments, the variant includes a mutation at an amino acid position corresponding to position 287 in SEQ ID NO: 11, wherein the residue in the parental laccase is substituted with a different residue. In some embodiments, the amino acid in the parental laccase is a small aliphatic amino acid residue. In some embodiments, the amino acid in the parental laccase is V, A, I, D, E, or P. In some embodiments, the amino acid in the parental laccase is V. In some embodiments, the substituting residue is P, H, or G. In particular embodiments, the mutation corresponds to V287P/H/G. In particular embodiments, the mutation corresponds to V287P, V287H, or V287G.

In some embodiments, the variant includes a mutation at an amino acid position corresponding to position 293 in SEQ ID NO: 11, wherein the residue in the parental laccase is substituted with a different residue. In some embodiments, the amino acid in the parental laccase is a small aliphatic amino acid residue. In some embodiments, the amino acid in the parental laccase is V, I, A, D, T, or N. In some embodiments, the amino acid in the parental laccase is V. In some embodiments, the substituting residue is N, T, or S. In particular embodiments, the mutation corresponds to V293N/T/S. In particular embodiments, the mutation corresponds to V293N or V293T.

In some embodiments, the variant includes a mutation at an amino acid position corresponding to position 319 in SEQ ID NO: 11, wherein the residue in the parental laccase is substituted with a different residue. In some embodiments, the amino acid in the parental laccase is a small aliphatic amino acid residue. In some embodiments, the amino acid in the parental laccase is V, G, F, T, N, or Q. In some embodiments, the amino acid in the parental laccase is V. In some embodiments, the substituting residue is W, T, or S. In particular embodiments, the mutation corresponds to V319W/T/S. In particular embodiments, the mutation corresponds to V319W or V319T.

In some embodiments, the variant laccase includes a mutation at an amino acid position in or near the active site of the enzyme. In some embodiments, the variant includes a mutation at an amino acid position corresponding to position 68 in SEQ ID NO: 11, wherein the residue in the parental laccase is substituted with a different residue. In some embodiments, the amino acid residue in the parental laccase is an aromatic amino acid residue, including or not limited to F, Y, W, or H. In some embodiments, the substitution is to a non-aromatic amino acid residue, i.e., A, V, L, I, G, M, S, T, D, E, N, Q, R, K, C, or P. In some embodiments, the substitution is to an aliphatic amino acid residue, i.e., A, V, L, I. In particular embodiments, the mutation corresponds to F68L.

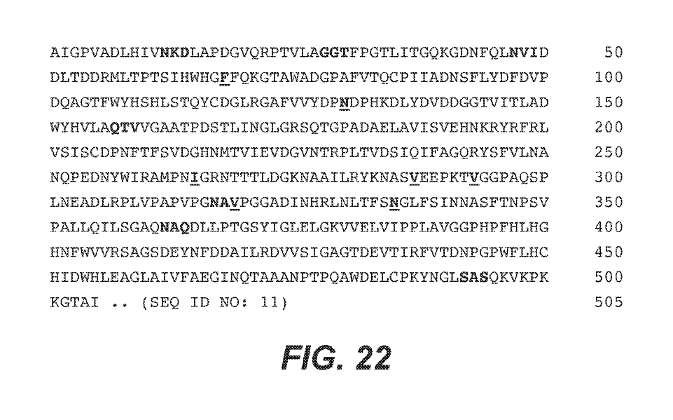

In some embodiments, the variant includes a plurality of mutations at amino acid positions corresponding to positions 68, 130, 265, 287, 293, 319, and/or 335 in SEQ ID NO: 11, which may be combined with a mutation that introduces an N-glycosylation site on the surface of the laccase enzyme. FIG. 22 shows the location of all the aforementioned mutations with reference to SEQ ID NO: 11; wherein exemplary N-glycosylation mutations are shown in bold, and surface charge and non-conservative hydrophobic residue mutations are shown in bold and underlined. In some embodiments, the variant includes a plurality of mutations at amino acid positions corresponding to positions 287, 293, and or 319 in SEQ ID NO: 11.

In some embodiments, the variant includes a plurality of mutations corresponding to V287G, V287P, V293T, and V319T in SEQ ID NO: 11. Exemplary combinations of mutations are as follows: I265R/V287G, I265R/V293T, I265R/V319T, I265R/V287G/V319T, I265R/V287G/V293T/V319T, I265R/V287P, I265R/N335R, I265R/N130E, F68L/I265R, F68L/I265R/V287G, F68L/I265R/V293T, F68L/I265R/V319T, F68L/I265R/V287G/V319T, F68L/I265R/V287G/V293T/V319T, F68L/I265R/V287P, F68L/I265R/N335R, and F68L/I265R/N130E.

Equivalents and variation on these mutations will be apparent to the skilled person in view of the present description.

### V. Mediators

In some embodiments, the present laccase enzyme systems, compositions, and methods, further include one or more chemical mediator agents that enhance the activity of the laccase enzyme. A mediator (also called an enhancer or accelerator) is a chemical that acts as a redox mediator to effectively shuttle electrons between the enzyme exhibiting oxidase activity and a dye, pigment (e.g., indigo), chromophore (e.g., polyphenolic, anthocyanin, or carotenoid, for example, in a colored stain), or other secondary substrate or electron donor.

In some embodiments the chemical mediator is a phenolic compound, for example, methyl syringate, or a related compound, as described in, e.g., PCT Application Nos. WO 95/01426 and WO 96/12845. The mediator may also be an N-hydroxy compound, an N-oxime compound, or an N-oxide compound, for example, N-hydroxybenzotriazole, violuric acid, or N-hydroxyacetanilide. The mediator may also be a phenoxazine/phenothiazine compound, for example, phenothiazine-10-propionate. The mediator may further be 2,2′-azinobis-(3-ethylbenzothiazoline-6-sulfonic acid) (ABTS). Other chemical mediators are well known in the art, for example, the compounds disclosed in PCT Application No. WO 95/01426, which are known to enhance the activity of a laccase. The mediator may also be acetosyringone, methyl syringate, ethyl syringate, propyl syringate, butyl syringate, hexyl syringate, or octyl syringate.

In some embodiments, the mediator is 4-cyano-2,6-dimethoxyphenol, 4-carboxamido-2,6-dimethoxyphenol or an N-substituted derivative thereof such as, for example, 4-(N-methyl carboxamido)-2,6-dimethoxyphenol, 4-[N-(2-hydroxyethyl) carboxamido]-2,6-dimethoxyphenol, or 4-(N,N-dimethyl carboxamido)-2,6-dimethoxyphenol.

In some embodiments, the mediator is described by the following formula:

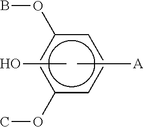

in which A is a group such as —R, -D, —CH═CH-D, —CH═CH—CH═CH-D, —CH═N-D, —N═N-D, or —N═CH-D, D is selected from the group consisting of —CO-E, —SO₂-E, —CN, —NXY, and —N⁺ XYZ, E is —H, —OH, —R, —OR, or —NXY, and X, Y, and Z are independently selected from —H, —OH, —OR, and —R; where R is a C₁-C₁₆ alkyl, preferably a C₁-C₈ alkyl, which alkyl may be saturated or unsaturated, branched or unbranched and optionally substituted with a carboxy, sulfo or amino group; and B and C are independently selected from Cₘ H₂ₘ₊₁; 1≦m≦5.

In some embodiments, A in the above mentioned formula is —CN or —CO-E, wherein E may be —H, —OH, —R, —OR, or —NXY, where X and Y are independently selected from —H, —OH, —OR, and —R, where R is a C₁-C₁₆ alkyl, preferably a C₁-C₈ alkyl, which alkyl may be saturated or unsaturated, branched or unbranched and optionally substituted with a carboxy, sulfo or amino group; and B and C are independently selected from CₘH₂ₘ₊₁; 1≦m≦5. In some embodiments, the mediator is 4-hydroxy-3,5-dimethoxybenzonitrile (also referred to as “syringonitrile” or “SN”).

Note that in the above mentioned formula, A may be placed meta to the hydroxy group, instead of being placed in the para position as shown.

For applications such as textile processing, the mediator may be present in a concentration of about 0.005 to about 1,000 μmole per g denim, about 0.05 to about 500 μmole per g denim, about 0.1 to about 100 μmole per g denim, about 1 to about 50 μmole per g denim, or about 2 to about 20 μmole per g denim.

The mediators may be prepared by methods known to the skilled artisan, such as those disclosed in PCT Application Nos. WO 97/11217 and WO 96/12845 and U.S. Pat. No. 5,752,980. Other suitable mediators are described in, e.g., U.S. Patent Publication No. 2008/0189871.

### VI. Utility

Industrial applications of laccases include bleaching and delignification of pulp and paper, drinking waste paper, textile color modification, decolorizing dyes, waste water treatment, depolymerization of high molecular weight aggregates, polymerization of aromatic compounds, radical mediated polymerization and cross-linking reactions (e.g., paints, coatings, biomaterials), activation of dyes, coupling of organic compounds, processing animal hides (e.g., de-hairing, liming, bating and/or tanning), keratinous fiber dyeing (e.g., hair and wool), in food or feed preparation or processing, or as an active ingredient in food or feed, and use in cleaning compositions, including detergent compositions, and generally for cleaning, disinfecting, decontaminating, and sanitizing. Additional uses are described in, e.g., U.S. Patent Publication No. 2008/0196173 and PCT Publication No. WO 2008/076322, which are incorporated by reference.

Particular textile applications include but are not limited to the treatment, processing, finishing, polishing, or production of fibers, yarns, fabrics, or garments, bleaching work-up processes, decolorizing of dye wastes, color modification (including by limited to bleaching) of dyed textiles, and the like. Color modification may be general (i.e., applied to an entire fabric or garment) or local (i.e., applied to only a portion of a fabric or textile). In some cases, it may be desirable to perform color modification simultaneously or sequencially with other enzymatic processing steps, such as abrading (e.g., using cellulases). Numerous uses are described in, e.g., U.S. Patent Publication No. 2008/0196173 and PCT Publication No. WO 2008/076322, which are incorporated by reference.

The present laccase enzymes can be used to decolorize any dye that can be decolorized using a laccase enzyme. Examples of such dyes include, but are not limited to, azo, monoazo, disazo, nitro, xanthene, quinoline, anthroquinone, triarylmethane, paraazoanyline, azineoxazine, stilbene, aniline, and phtalocyanine dyes, or mixtures thereof. In some embodiments, the dye is an azo dye (e.g., Reactive Black 5 (2,7-naphthalenedisulfonic acid, 4-amino-5-hydroxy-3,6-bis((4-((2-(sulfooxy)ethyl)sulfonyl)phenyl)azo)-tetrasodium salt), Reactive Violet 5, methyl yellow, congo red). In some embodiments, the dye is an anthraquinone dye (e.g., remazol blue), indigo (indigo carmine), or a triarylmethane/paraazoanyline dye (e.g., crystal violet, malachite green). In various embodiments, the dye is a reactive, direct, disperse, or pigment dye. In some embodiments, the dye is comprised within an ink. In some embodiments, the dye is indigo and/or a sulfur-based dye. In some embodiments, the textile is denim dyed with indigo and/or a sulfur-based dye. In a particular embodiment, the textile is dyed with indigo, and the laccase enzyme and mediator are used to oxidize the indigo to isatin.

Various aspects and embodiments of the present compositions and methods are further described in the following numbered paragraphs:

1. A variant laccase enzyme derived from a parental laccase enzyme is provided, the variant laccase enzyme having:

(a) a mutation at a position corresponding to position 68 of the amino acid sequence of SEQ ID NO: 11;

(b) a mutation that alters the surface charge of the parental laccase enzyme;

(c) a mutation that alters the surface hydrophobicity of the parental laccase enzyme; or

(d) a mutation at an amino acid position corresponding to a non-conservative, hydrophobic amino acid residue located on the surface of the parental laccase enzyme;

wherein the mutation is a substitution to a different amino acid residue compared to the parental laccase.

2. In some embodiments the variant laccase enzyme of paragraph 1 has a mutation at a position corresponding to position 68 of the amino acid sequence of SEQ ID NO: 11,

wherein the mutation is a substitution of an aromatic amino acid residue to a non-aromatic amino acid residue.

3. In some embodiments, the variant laccase enzyme of paragraph 2 has a substitution of an aromatic amino acid residue to an aliphatic amino acid residue.

4. In some embodiments, the variant laccase enzyme of paragraph 3 has a substitution of an aromatic amino acid residue to A, V, L, or I.

5. In some embodiments, the variant laccase enzyme of paragraph 4 has a mutation equivalent to F68L in SEQ ID NO: 11.

6. In some embodiments, the variant laccase enzyme of paragraph 1 has a mutation that alters the surface charge or alters the surface hydrophobicity of the parental laccase enzyme, wherein the mutation is at a position equivalent to position 130, 265, 287, 293, or 319, in SEQ ID NO: 11.

7. In some embodiments, the variant laccase enzyme of paragraph 1 has a mutation that alters the surface charge or alters the surface hydrophobicity of the parental laccase enzyme, wherein the mutation is at a position equivalent to position 130 in SEQ ID NO: 11.

8. In some embodiments, the variant laccase enzyme of paragraph 1 has a mutation that alters the surface charge or alters the surface hydrophobicity of the parental laccase enzyme, wherein the mutation is at:

(a) an amino acid position equivalent to position 130 in SEQ ID NO: 11, wherein the residue in the parental laccase is substituted with a different residue selected from D, E, R, and K;

(b) an amino acid position equivalent to position 265 in SEQ ID NO: 11, wherein the residue in the parental laccase is substituted with a different residue selected from R, H, and V;

(c) an amino acid position equivalent to position 287 in SEQ ID NO: 11, wherein the residue in the parental laccase is substituted with a different residue selected from P, H, and G;

(d) an amino acid position equivalent to position 293 in SEQ ID NO: 11, wherein the residue in the parental laccase is substituted with a different residue selected from N, T, and S; or

(e) an amino acid position equivalent to position 319 in SEQ ID NO: 11, wherein the residue in the parental laccase is substituted with a different residue selected from W, T, and S.

9. In some embodiments, the variant laccase enzyme of paragraph 1 has mutations equivalent to:

(a) I265R/V287G,

(b) I265R/V293T;

(c) I265R/V319T;

(d) I265R/V287G/V319T;

(e) I265R/V287G/V293T/V319T;

(f) I265R/V287P;

(g) I265R/N335R;

(h) I265R/N130E;

(i) F68L/I265R;

(j) F68L/I265R/V287G;

(k) F68L/I265R/V293T;

(l) F68L/I265R/V319T;

(m) F68L/I265R/V287G/V319T;

(n) F68L/I265R/V287G/V293T/V319T;

(o) F68L/I265R/V287P;

(p) F68L/I265R/N335R; or

(q) F68L/I265R/N130E;

in SEQ ID NO: 11.

10. In some embodiments, the variant laccase enzyme of any of the preceding paragraphs is derived from a parental laccase is obtainable from a Cerrena species.

11. In some embodiments, the variant laccase enzyme of any of the preceding paragraphs is derived from a parental laccase is obtainable from Cerrena unicolor.

12. In some embodiments, the variant laccase enzyme of any of the preceding paragraphs is derived from laccase D from C. unicolor.

13. In some embodiments, the variant laccase enzyme of any of the preceding paragraphs is derived from a parental laccase having an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

14. In some embodiments, the variant laccase enzyme of any of the preceding paragraphs has an amino acid sequence that is at least 70% identical to the amino acid sequence of SEQ ID NO: 11.

15. In some embodiments, the variant laccase enzyme of any of the preceding paragraphs has an amino acid sequence that is at least 80% identical to the amino acid sequence of SEQ ID NO: 11.

16. In some embodiments, the variant laccase enzyme of any of the preceding paragraphs has an amino acid sequence that is at least 90% identical to the amino acid sequence of SEQ ID NO: 11.

17. In some embodiments, the variant laccase enzyme of any of the preceding paragraphs has an amino acid sequence that is at least 95% identical to the amino acid sequence of SEQ ID NO: 11.

18. In some embodiments, the variant laccase enzyme of any of the preceding paragraphs further comprises a mutation that introduces a glycosylation site into the amino acid sequence of the parental laccase.

19. A composition comprising the variant laccase of any of the preceding paragraphs is provided.

20. In some embodiments, the composition of paragraph 19 further comprises a chemical mediator.

21. In some embodiments, the composition of paragraph 20 comprises a phenolic compound chemical mediator.

22. In some embodiments of the composition of paragraph 21, the chemical mediator is a phenolic compound is selected from the group consisting of syringonitrile, acetosyringone, and methyl syringate.

23. A method of bleaching a surface comprising contacting the surface with a composition of any of the preceding paragraphs is provided.

The following Examples are provided to further illustrate the present compositions, methods, and systems, and should not be construed as limiting.

### EXAMPLES

### Example 1

### Creation of Trichoderma reesei Screening Strains

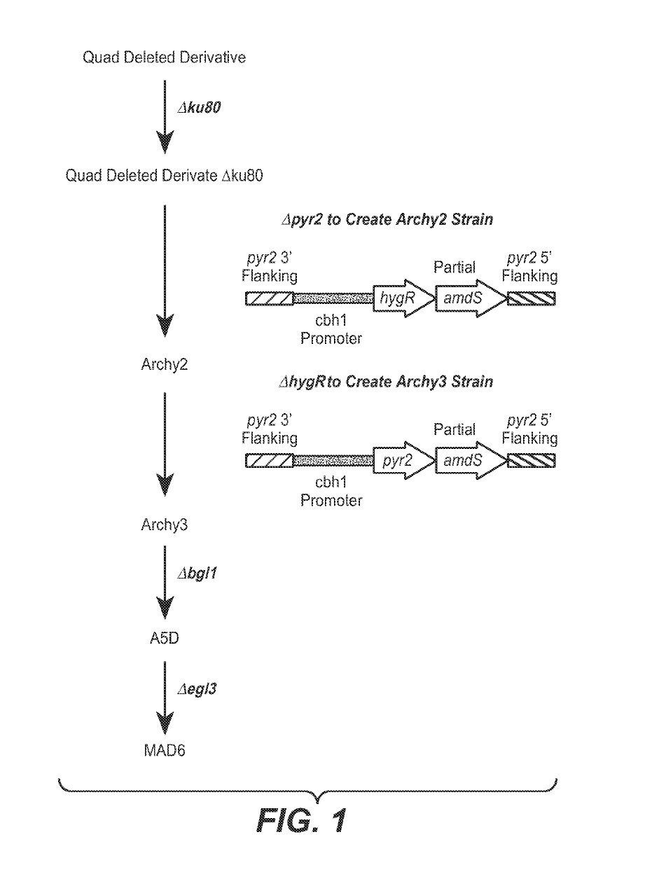

Improved screening strains were created to increase the consistency of CBH2 variant expression in the presence of factors unrelated to the amino acid sequences of the enzyme variants. In particular, T. reesei screening strains were developed in combination with a targeting vector to force integration of cbh2 variant genes (e.g., coding region in operable combination with a regulatory sequence). The new strains prepared during development of the present disclosure, combine several mutations that are advantageous for screening variant libraries. A schematic of the genetic engineering steps is shown in FIG. 1.

Deletion of ku80 from the T. reesei quad deleted derivative strain. A single orthologue of MUS52, the N. crassa orthologue of the human KU80, was identified by TBLASTN search in the genome sequence of H. jecorina QM6a (T. reesei) and was consequently named T. reesei ku80. protein id 58213; http://genome.jgi-psf.org/Trire2/Trire2.home.html The nucleotide sequence of the T. reesei ku80 gene is provided as SEQ ID NO: 37:

ATGGCGGACAAGGAAGCAACCGTCTTCATCATCGACCTCGGCGCGTCCAT
GGCAGCTGTCAATGGGGGTCGAGAAGAATCCGACCTTGATTGGAGCATGA
GCTACGTCTGGGACAAGATCAGCAACGTCGTGGCCTCGAATCGCAAGACG
CTGTGCGTTGGCGTCGTGGGGTTCAGAACCGACGAGACAAACCACACGCT
GAGCGAGGATGGGTACGAGAACATCTCCATATTGCAGCCCCTGGGGCCGA
TGAGCATGTCCAGCCTCAAGGCTCTTCAGCCCAAGGTGAAGCCGAGCAGG
ACGGTGGAAGGCGATGCCATCTCGGCGATTGTCATTGCCGTCGACATGAT
TGACAAGTACACGAAGAAGAACAAATGGAAGCGGCAGATTGTTCTCATTA
CCGACGGCCAAGGCGAGATTGATCCAGATGATATTGGCGACATTGCTAGA
AAGATGCGCGACTCGAATATTGAATTGACAGTCTTGTGAGTTGGCGAGAC
CGTTTGGCGGACGGTAATGGTGCTGACGGTGATGCAAGGGGCGTCGACTT
TGATGCTCCCGATTACGGCTTCAAAGAGGAGGACAAACCTTCAGTCAAGG
TACTCCATATGTTCACTTCTTTTCTTTTTCTTCTTTATTTTCTTTTCTTT
TGAAGCTTTCATTAACCTCTTCGTTAGAAGCAAAACGAAGAGACCCTAAA
AAAGCTCGTGGATGGCTGTGGCGACGACTCAAGGTTCGCCTCCATGGTCG
AGGCCATTGACGACTTGAATGAGCCACGAGCAAAGTCGGTCAAGCCTTAC
AAAACGTACGAAGGTCTCTTGACCTTGGGAGATCCGAAAAACGCTCCCGC
AGTGGTGGAAATCCGCGTCGAGAGATACTTCAAGACCCATCTAGCCAGGC
CACCTGCCGCCAGCACCGTGGTGGTCAAGGAGGAGCAAGCTGGGCCGTCT
CAGGCAGACGAGGACGAACAGATGGACGGAGCGGAACTTACAGCTGTGAG
GCAGGCCAGGACATACAAGGTCAATGATCCAGATGCCCCTGGCGGTAAGC
GTGACGTTGAGTTTGAGTCTCTGGCCAAAGGGTACGAGTACGGCAGGACG
GCAGTCCACATCAGCGAGTCTGATCAAAACGTCACCAAGCTCGCGACAGA
AAAGAGCTTCAAGATCATCGGCTTCGTCCAGAAAGAAAAGGTATTGGCTT
GGCTCTCAGCATTTGACCCGTTGCTCTTGGCTAACCCTTGTTTAGTATGA
AATGCTCCTTAATCTTGGCGAAACCTGCGTTACCGTTGCATCCAAGTACG
ATGAAAAGTCTGAGCTGGCTTTTAGCTCTCTGGTGTGGGCGCTCTCGGAG
CTCGACGCCTACGCCGTGGCCCGCCTAGTAACTAAGGACCAAAAGGACCC
CATGCTGGTGTTACTGATGCCGTATATGGAGCCTGATTATGTTTGTCTCT
ATGATGTGCCTCTGCCTTTCGCAGAGGACATCAGGACGTACCAGTTTCCT
CCCTTGGACAGAGTCGTTACCGTCAGTGGCCAAACGCTCACCAACCATCG
CCTATTGCCATCCGACGAGCTCAACCAAGCGATGAGCGACTACGTAGATG
CCATGGACATTTCAAGTTATGGTATCGATGAAGATGGGTGAGTATAGAAG
ATGATTGTTCAAATCTTTCACTTCTAAGCATTGCTTCTGATCTAGGCAAC
CGGCTGAATATGCCACCATCGATGAGTTATACAACCCTGCGATACATCGC
ATAGGCCATGCGATCAAACAACGAGCGATCCACCCAGAGAAACCCGTGCC
CGAGATCCCCCCAGTCTTGCTTAGATTCGCAGCACCCCCGACAGAACTCG
TCGAGACTGTGCAGCCTCATATCGATGCACTGATTCACGCTGCAGACGTG
AAGAAAGGTACTGATTCCATTACATATGCTTCTCTGCACACTGATGTTTG
ATTTGTGCTAACGCCCCCCTTAGTGCCGCCCAAGGCCAAGGGCAAGCGCC
AAAGAGAAACAGTTAAACCCATCTCGGGACTGGATGTGGATGCCCTTCTG
GGAGAAGAGCAGAAAGGTTCCATTAGTCCGGAGAATGCCATTCCGGACTT
CAAACGAGCCCTCAACTCGTCCGAAGAAGTCGAGCAGATTGCCGACGCCA
CAAAACAAATGGGGGCCATTGTGCGGTCTCTCATTACGGACAGCTTCGGG
GATAGCAAATATGCCCAGGCAATGGAAGGCATTGGTGCGATGCGTGAGGA
GCTGATCAACCTGGAAGAGCCTGGCCTGTACAACGACTTTGTGCGCGACT
TGAAGAAAAGTTTGCTATCTGGAGCCTTGGGTGGTGACAGGCGAGATTTC
TGGTTCAAGATGAGGTGGGCGAAGCTGGGCCTGATTGACAAGAAACAGTC
GGAGGTGTCTTCGGTCACTCTTGAGGAGGCGGACGAGGTGAGTGGTGCAG
CATGCTGTCGGATTATACGGACGTTGTTTGCTAACTTGTGGGATAGTTTT
ACAAGTCGAGGTGAGGTATCTACGTTGACCAAGAATGGGACCATGTATAT
GAGCGGTGTAACAACAGAATCCTGTGCTTTGAGCATTGTATGA

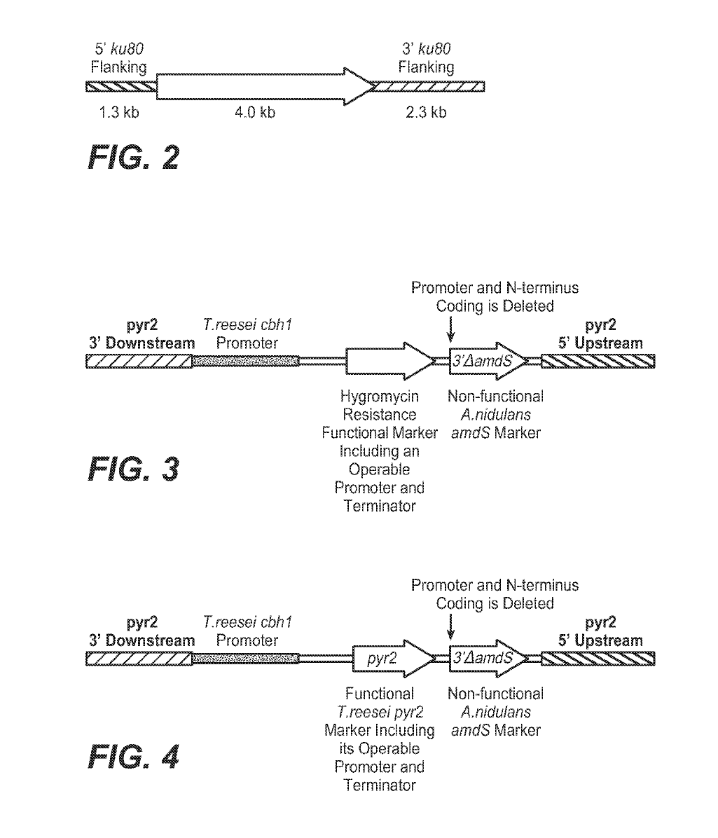

The T. reesei ku80 gene was deleted from the quad deleted derivative strain using standard methods of the art (WO 2005/001036). Briefly, a ku80 deletion cassette was utilized that employed a selectable marker flanked between 1.3 kb of 5′ ku80 sequence and 2.3 kb of 3′ ku80 sequence, as schematically shown in FIG. 2. The variant T. reesei als, which confers resistance to the herbicide chlorimuron ethyl, was used as selectable marker as described in WO 2008/039370. The nucleotide sequence of the ku80 knockout cassette is 7685 base pairs in length: bases 1-1271 correspond to the 5′ ku80 homologous region; bases 1280-7685 correspond to the als-chlorimuron ethyl resistant variant (A190D); and bases 5381-7685 correspond to the 3′ ku80 homologous region. The nucleotide sequence of the ku80 knockout cassette is provided as SEQ ID NO: 38:

GGCCGCCTCAACACCCACACTCGAGGCACACGAGTTCATCGGCGGCTTCC
CCCACAAGCTCTCGGCCAACCTGCTACCGGCTCTCTCGCGAGACTTCCCA
AAGCCTACAAACGAGGTCGACGTCAAGGAGGCCCTCGAGCGCCAGCCCGG
CAGATGGAGCCTCCAGGGCCAGATCAAGGCCAACAACATGAGAGCCCAGA
GCGCCGCACTCCGGCTCGACGACAAGGAGGGCAAGGCGAGAGCCTTTGAG
GAGGCCAAGCGCGAGCTACTGGCGTATCACCACAGCGCCCTGCGGAAGCC
TTCCGGCGCAAGATAATGAGCTTGATCGCAATGACGAGTTCACGTACGCT
TTGCCATATTGTTGTTGCTTTTTGTTTGGTCCTACATGTACGGCGCATTG
GTTGGGAGGATATACCCACGGAGAGTGTCCGAGTGGCTTCTGGGATTTAG
AGCGTCATTAGCAGGATAGAGATGGTTGGCCAGGGGAATGGAATTGACTT
TTCACTACAAGGAACTTGTTCACTCTGGTGTTGATTCCCATTGCGTGACT
GGTAGTAGGGAGGAATGCTTTTACTTTGTGCCACTAGACCGCAGAGAAGG
GTTGGTTGCAAGCGGGGTCCGTGTATACCGACCAAGAGTGATGGGCATAC
AGCAACGTTTCTGAACGACTTCATTTTGTCCGAGTCTACTGGATGCGAGA
TGCCAGCGTGAAGCCGTACGCCACCAGGGCGACGAACTCGACAAGGTTGA
CGAGGGAGGAGATGCCGTGCAGCATGCCAAACTTCTTGTTGAGGGCACGC
ATCTCATCCGACTGTGCATCCTTGTCATACCACTCCTTTCCGTCTCGCTT
GGCTGGTGGGAGGGTTCAACAAATCCATCGTCAGCCATCCGGGGTCTCAA
ATCAATGGCGTGCATGCGGAGTCGGGCTTGAGGCTAACCTTGTCCATGGC
GGTCCTTCATGGTCTTGACAGTGGCGGGAAGCAGCACGGCGAGGTTGACG
AGGCCGCTGACGAACATGGTTGCGATGGGCACCAAGGAGCTCCACTTGTT
GGGAGCGTCGACGAGGCCGCCGATGCCGCCCTTGATGCCCAAGAGGGCGT
TTCCGGGGAACGTGAGGGCGAGCAGCGCGGGGATGGCCGTCTGCATGCCA
AAGTAGATGGGGAACAGCTTGCTCTGGATGGCGGAGAAGGAGGGCCGGCT
GACGGTGCGGAACATGACGATGCCGTTGACGAAGGACTGCAGTAGCGTAG
TGTGATGGTAAGCAGCTGGCCGGCGCGCCTGAGACAATGGCCGGCAATGG
TAAAAAGGACCAAGATGTACTAGGTAGTTGCAATGTGGCTTATTACCTAC
CTACTACCTGGTAGGCACCTACTAGGTACTTGGGTAGACGGACAATGAAA
TTTGAAGTCGGGGTTGCAGGAAAGCAGGGCGCTGGACACATTGTGCTTCA
GGCGGTACCCGTCGTCATCGTCAGCCAATGTCGAGGCCCGGCAGCCCGAG
GAGCGAGACAACCTTGGCCGGAGGAGCCCGCAGGTACCTGCCAAAGCGCG
GCTGGTACCTCTCAACCCTCTCAGGCCTGTTGGATGCCCTATGACATGCC
CTGGGGGATGCAGCTGTTGCCCCGGCCCCGCACTTTCGGGTGACCGCGAG
GCTGCTGATTGGCTGGTTGCCACGGGCTGGGCGGTCCCTGAAGTTGTTGC
CATCTGAACTCTGTCGGCGCTGGCGTCGGCTGCGCCCAATGGGAGGCGAG
ACAACTCAGGGTACTAGAATCACTGACAGAAGAAGAGAATCGAAAGTAGG
TAGACAGCCAATTCGTTGCATGGCAGGCAACCGCACAGGAGAAAAATTGA
CTACCCCACAATCAGGCACAGTAAGTAGGGCACAGTACGTATGTACAGAC
AAGGCGCAAGCGATACTGCGCGACCCGGTACCTCGCCGGCTTGACACGTG
CGACAGGCTACTTTACTAGTATTCGCAGCGGCGGGTCGCGCATTATTACA
TGTACTGTGCCGCCATTTGATGACTGGGCTGCTGCAGTATTAGTAGATCT
GCCCGGCATCGCCCTTCCATGGGCGCGACCCGGGACTGGACCCTCTGACT
CTACCTACATGTACCTAGGCCGGGCCGGGCTTGGTGACTTTTGTCCGATC
AGGTCGTTCGCCTGGCTACCTATTATTTCTCTTTCTTCTTCTCCATCCTG
CTTCTGGCCTTGCAATTCTTCTTCGCCACTCCTCCCTCTTCCCCCCGCGA
TACCCTTGAATTCGTCAGAGAGGAAAAGACGAGAAAAAAAAGGGCAGCAG
AGACGTCGGTCTGGCTCACGTGCTGCATCTCTGCGCACTCTCATTTTTTT
TATTGTCCGACCCCTCCCTCAACCTTCTCCTTCGTTGACAGGCTAAGCCT
TGCTTCGACGCTCTCTCTTTGAATTTTTCTACTTCTACCTTCTTTTCTTG
CGTGTTACCCACCATAGCTCGATTCACGATGCTCCGAAGTCGCCAAGTCA
CAGCCAGGGCCGTCCGGGCTCTGGGCCAGGCGCGCGCCTTTACCTCGACG
ACCAAGCCTGTCATGATCCAGAGCAGCCAGAGGAAACAGGCCAACGCCAG
CGCTGCTCCGTAAGTCGCCCATTGCCATTGCATCTTCTGTTTGATATATA
CTTCCTGCTGCTTGCGTGGCGTCGTCTCTCGGTTATGCGTGTCAAGGACC
AGGTGTGTTCGCATCGTGGTTTTCCAGCGCCGATTACCGGGGGACGAATT
TTTGGCTGCTCAACTCGCGCGCGCGCATTCTGATTCTTCGTTTTCAATCT
TGAGCGACAACTGGCTAACATAATGGCCATTGGCAATTGCTTCACACAGA
CAAGTCCGCCCTGTACCGAGCCCTGCTTTCAACGCTGAAGACAAAGACCG
CAGCCATGTGCAGCCTCTGGTCAACCCGTCGAAGCCCGACATGGATGAAT
CGTATGTCCACGTCCCCTCGTCCCGCCCTACAAAATGAACACGATTACAC
CAGAATTTTTGCAACAATCGACACTTCTATAACAGACCAATTGAGCTTTG
TTCTGACCAATCATGTTGCTCTAGATTCATTGGCAAAACCGGAGGCGAAA
TCTTCCACGAGATGATGCTGCGACAGGGTGTCAAGCACATTTGTAGGTTC
CGATGCCGGCCGCCCACACGGGCTCCATCCTTGCTCCATCTCTCCAGCTA
GGCAAATCTCGCTAACCTTGAGTCACCATCCAGTCGGATACCCTGGCGGC
GCTATCCTGCCCGTCTTCGACGCCATCTACAACTCAAAACACTTCGACTT
CATCCTGCCCCGTCATGAGCAGGGAGCTGGCCATATGGCCGAGGGCTATG
CCCGTGCCTCGGGCAAACCCGGTGTCGTCCTGGTGACTTCCGGCCCCGGT
GCTACCAATGTCATCACGCCCATGCAGGATGCCCTGTCGGACGGAACGCC
CTTGGTCGTCTTCTGCGGCCAGGTCCCCACCACGGCCATCGGCAGCGATG
ACTTCCAAGAGGCCGACGTCGTGGGCATCTCGCGGGCCTGCACCAAGTGG
AACGTCATGGTCAAGAGCGTTGCTGAGCTGCCGCGGAGAATCAACGAGGC
CTTTGAGATTGCCACCAGCGGCCGCCCTGGCCCCGTCCTCGTCGACCTGC
CCAAGGATGTCACGGCTGGTATCCTGAGGAGAGCCATCCCTACGGAGACT
GCTCTGCCGTCTCTGCCCAGTGCCGCCTCCCGCGCCGCCATGGAGCTGAG
CTCCAAGCAGCTCAACGCCTCCATCAAGCGTGCCGCCGACCTCATCAACA
TCGCCAAGAAGCCCGTCATCTACGCCGGTCAGGGTGTCATCCAGTCCGAG
GGCGGCGTTGAGCTCCTGAAGCAGCTGGCGGACAAGGCCTCCATCCCCGT
CACCACCACCCTCCATGGCCTGGGTGCCTTTGATGAGCTGGACGAGAAGT
CGCTGCACATGCTGGGCATGCACGGCTCGGCGTATGCCAACATGGCCATG
CAGCAGGCCGACCTCATCATCGCCCTCGGCAGCCGATTCGACGACCGTGT
TACTCTGAATGTCTCCAAATTTGCGCCTGCAGCCAGGCAAGCTGCTGCCG
AGGGCCGCGGCGGCATCATTCACTTTGAGATCATGCCCAAGAACATCAAC
AAGGTCATCCAGGCGACCGAGGCCGTCGAGGGCGACGTCGCCACCAACCT
GAAGCACCTCATTCCCCAGATTGCCGAAAAGTCCATGGCGGACCGAGGAG
AGTGGTTCGGCCTCATCAATGAGTGGAAGAAGAAGTGGCCCCTGTCAAAC
TACCAGCGCGCGGAGCGGGCTGGCCTCATCAAGCCGCAGACGGTCATGGA
GGAGATTAGCAACCTGACGGCCAACCGAAAGGACAAGACGTACATTGCCA
CGGGTGTCGGCCAGCACCAGATGTGGGTTGCCCAGCACTTCCGCTGGAGG
CACCCTCGATCCATGATTACCTCTGGTGGTCTGGGCACCATGGGCTACGG
TCTCCCCGCGGCCATTGGCGCCAAGGTGGCCCAGCCCGACGCTCTCGTAA
TTGACGTTGATGGCGATGCCTCGTTTAACATGACGCTGACGGAGCTGTCG
ACTGCTGCACAGTTCAACATTGGCGTCAAGGTGGTTGTGCTCAACAACGA
GGAGCAGGGCATGGTGACGCAGTGGCAGAACCTCTTTTACGAGGACCGAT
ATGCCCACACGCACCAGAAGAACCCCGACTTCATGAAGCTGGCCGACGCC
ATGGGCGTTCAGCACCAGCGCGTGACGGAGCCGGAGAAGCTGGTCGATGC
CCTGACGTGGCTGATCAACACCGATGGCCCGGCCCTGTTGGAGGTTGTCA
CGGACAAGAAGGTGCCTGTCCTGCCCATGGTGCCCGCCGGATCGGCCCTG
CACGAGTTCCTCGTCTTTGAACCTGGTGAGTCTACTTCAGACATATTGCT
TGCGCATTGCAGATACTAACACTCTCACAGAAAAGGATAAGCAGCGCCGT
GAGCTGATGAAGGAGAGAACAAAGGGTGTGCACTCCTAAAGCGATGATGT
CTGCGAGGGGTTCTTCGTTGAACCCTAGTTCAGGCACCATCTTACCCTCT
TATTTTTTCCCGTGGGCTTTCATTTTGTGTCATCCGAGCATGACGTTGTA
GGGTTGGAGTTTCTTCCTTTTTATCTTGTCATTTACTGGTACCCATAGGC
GCGAGACTAGGCTTCCATGTTTTGTTTTGCGACTTTCAAAAAGTACTTTT
AGTGGTTTGGGGCACGACGAGGGGGGGCAACCTCTTCTGTCGAAAAAGGT
GGCTGGATGGATGAGATGAGATGAGATGAGGGTGAAGATAGATACCTGCA
GTGTTTTTGACGCGACGGGATGGCGATCGCAGCACCCCCGACAGAACTCG
TCGAGACTGTGCAGCCTCATATCGATGCACTGATTCACGCTGCAGACGTG
AAGAAAGGTACTGATTCCATTACATATGCTTCTCTGCACACTGATGTTTG
ATTTGTGCTAACGCCCCCCTTAGTGCCGCCCAAGGCCAAGGGCAAGCGCC
AAAGAGAAACAGTTAAACCCATCTCGGGACTGGATGTGGATGCCCTTCTG
GGAGAAGAGCAGAAAGGTTCCATTAGTCCGGAGAATGCCATTCCGGACTT
CAAACGAGCCCTCAACTCGTCCGAAGAAGTCGAGCAGATTGCCGACGCCA
CAAAACAAATGGGGGCCATTGTGCGGTCTCTCATTACGGACAGCTTCGGG
GATAGCAAATATGCCCAGGCAATGGAAGGCATTGGTGCGATGCGTGAGGA
GCTGATCAACCTGGAAGAGCCTGGCCTGTACAACGACTTTGTGCGCGACT
TGAAGAAAAGTTTGCTATCTGGAGCCTTGGGTGGTGACAGGCGAGATTTC
TGGTTCAAGATGAGGTGGGCGAAGCTGGGCCTGATTGACAAGAAACAGTC
GGAGGTGTCTTCGGTCACTCTTGAGGAGGCGGACGAGGTGAGTGGTGCAG
CATGCTGTCGGATTATACGGACGTTGTTTGCTAACTTGTGGGATAGTTTT
ACAAGTCGAGGTGAGGTATCTACGTTGACCAAGAATGGGACCATGTATAT
GAGCGGTGTAACAACAGAATCCTGTGCTTTGAGCATTGTATGATATGATT
ATTGATGAACCGGACAAAAGGGGGTAGGGGATTGATGCCATCACGACCGA
TTGACCAGACCTGGATTCTCGCACAGCATGGCTGCTGATTTTGTTGACCT
TGCGACGTAACATCCCTGAAGAACAACCTACTATTAACCTATCATTTAGC
AGAAGCTCTGTAACCTTCTTGATTCTTGTATTCAGCTTCTGAGTCTGTCA
AATGTAATCATTTCGAGGTTGTGTAATTCCGGCCAAGCAGGCGGCCGTCT
GCCAGCGCCTGCCTAGGCTGCACCGCAATCTGCCCAATCAGCTGCCCTTC
AGTTTCGTTTGACCTTGCAGCTGCCCTTCATCCTTTATCTGCACACAATT
CTTTTTCCTCTGCTCTGCGCATTCTTCTCTCTCTCGTCTCCCTTCTCAAG
CTCAACTTCACCTCATCCGCTCCACTACAAGCCCTCCCGTCGTCGTCTCG
CATCCTCATCTCGACTGCGGCCAGCAAAACAAGCAAAGCCGTGATCGATC
CTCAGCATGGCTACCTTCAACCTCACCGTCCGCCTGGAGATGCTCAAAGA
AATTGGAATCACCGTCCAATACGGCGAGCATGTAGCGAAAGAAGCAGCCA
GCAACGAAGCAGCGATGGCATTCGAAGAAGAAGAAGAGTTCCCCGCCGTT
GTGCCGCCCAAGGCAGAACAGCACGCCTCTGAACACGACGCTGGCCACGA
TGCTTGGGACGCGGCTGCCCACATCTCGACTTCGGCGCAAGAACAGCAGA
AGCCCCAGGAGATGGACGACTCGTCTATCGTGATGCCGCTGGACTACTCC
AAGTTTGTCGTTGGAGAGCCTGCGGACGAATCCATCAGCTTTTGCTCGTG
GAAGGTCGTCGAGGCTTATCCTGACCAGTTTATCGGCAAGGCAAACAGGC
CTCGTGTATGTAGCGATTGCTTTCTCTGCATTATGGGAATCTCAAGAGAG
TATGGTAGAAGATAACTGACAACTTGCAGGCCAAGCCGTACTTTGACAAG
ATTTTGGAAGACAGAGTCTGGGATTTGTGAGGATCTTGATTGATGTGCAT
ATGGCGACATGCCTGCTAATATCATTGTAGCTTCTATCTCTACAACCCCG
AGAAGCCTTCAGAGAAGCCTCGCGTGCTGGTGCCCACTGTTCAGCTCGAA
GGCTTTCTCAAAAGCATCAACAGAGCGCTCGGTACTTCTCTCACCATTCC
AGGAGGGGCAAACCAGGACCGTTTTTATCTGAGGTTCGGCCAGGGAGACA
CCCCAAGGCCTCGATATCTACAGAGGTCGAGAGACCAGAAATCCCTAAAG
ATTGAAACGTTCCCCGATTTTCAACAGGCGGACTACGACAGCTTTAGGAA
CGCGCATGGCGCCATCCAGGAGGACTGGTTGAAGAACTGGCAGATGCTGG
TACCTCGGCCGAGTTTCGACAAGAAGAAAAATGCAGACAAAAGAGCAGCC
AAGAGAAGGCTCGAGCGAGAGCGAATGCTTCACAATACGCAGGAATTTCT
TCATTTGGCAGGTAAGGGCAAAGGGGCTGACGTGG.

Creation of the Archy2 strain from the T. reesei Δku80 quad deleted derivative strain. The pyr2 gene was deleted from the ku80 knockout strain. The pyr2 deletion cassette contains the T. reesei cbh1 promoter, a hygromycin resistance gene and a partial amdS selectable marker flanked by 5′ and 3′ pyr2 sequences, schematically shown in FIG. 3. Use of this vector permits screening for resistance to hygromycin and fluoroorotic acid of pyr2 knockout transformants. The partial amdS gene contains the 3′ portion of the gene, but lacks a promoter and the amino-terminal portion of the coding region, and is consequently non-functional. The nucleotide sequence of the pyr2 knockout cassette is 9259 base pairs in length: bases 1-1994 correspond to the pyr2 3′ homologous region; bases 2002-3497 correspond to the T. reesei cbh1 promoter; bases 3563-5449 correspond to the hygromycin resistance selectable marker; bases 5450-7440 correspond to the A. nidulans amdS 3′ partial marker; and bases 7441-9259 correspond to the pyr2 5′ homologous region. The nucleotide sequence of the pyr2 knockout cassette is provided as SEQ ID NO: 39:

ATCACGCCCTCGCATAAAAGACCCTCAAGAGTCCATGTGCCCTATCTGCC
TGATCTTCCTAACCCTTATTTAACATTGGCCCTATCACAACCTAGTTCTT
CTTCAGCCTGCTTTGTCAACACTTGTCACGGTTCAACTCAACGTAATCAG
CAGGTAGCAGGACGAGGATAGGGAGAGAAACGAAGAGAAGAAGAGGAGAG
AGGAAGAAAAAAAAAAGAAAAGAAAGAAAAAGGGAAAAGGAAAGAAGGAG
GAAAAGAGAAGAAAGTCAGATGAAGAAGCAAGAAGACGCCATGGTAGCCA
CCGCTTGTCAGGGCTCCTTAGCAACGAACAACTCTAGCTTGGGGACTTGT
CGATGTGTCGTTTCCTTCCTACCCATCAGCACCAACGATGAGTTCGATAT
AGACGAGGACCTCATGGAAGTAGAGACCATTGGGTTCGACAGGATCTCTC
AGTTTCACTTCTATGAGGTCTGTCGCTCGGATGACTTTTTGAGGAGCTTC
CCCTTCTGCTTCAACCCCAAACTCTCTTTCCTGAAACCGCAGCACGTTGG
CACGGCCGTGTTGCTGGAGCAGTTTGCTTTCGAGCACTCTCAGCGTGGTT
TCAGCAGCCCACTGGTGAGTGGCCTCCTTTGACGTCCACACCTTGCTCCT
GTCGCATGCGTATCTGGTGGGAACGACTGCTCCAAGGAGGATTGCTAACG
AGGTTGTAGGCCGAATATCGCATCAGATTCTCCGGTAACCTTAGCTACGG
CCTCTTCAACATCTGTGACATGACGGAGCGCAAGTACTGGTGGTTGGCGA
CCAAGATGCGCGGCTGGAACATCGACGGCTGCCCCGAAGACGTCAGGAGA
CTCATGTTTGTTCACATCATCGCCACCCTGGGATGCAGCCCCGTCGTGAC
GGATGAAGACATGGACTACCCCAAGAACTGGGCGGCAATTCTCCACGGTA
GAGACAGATATCCGAGTGAACCTGTGGGCCACCGGCCTCATGGGCGCACC
ATCTGCCTCCACTCGGTGGCCGTCTGCCCTCGTCTCCAGGGCTTGGGTCT
CGGTACTGCGACTCTGAAGTCGTATGTGCAGCGCATGAACAGCCTCGGCG
CCGCGGACCGTGTTGCTCTCGTTTGCCGCAAGCCCGAGACGAGATTTTTT
GAAAGATGCGGCTTCAGGAACAGCGGCCGGAGTAGTATCAAGACTCTGGT
CGGCGAATACTACAACATGGTGTGTGCTTCCACATCGACTTGGCCAGACT
CTATACGATTTTCAAACCTCGCTATACGTCATATTGACTTGTTTCTTTAG
GTCTTCGATTTGCCCGGGCCCAAAGACTTTATCGACTGGAATAGCATTGC
CGACGCTGCCAAGAAGATGTGAACCATTTGACTGATACGATGTGTGCTAC
GCATGTCGACCTTCTTTGTTTGTTTCTTTGGCGGCTCTTTGTATACCTTG
GGACACGGCAGACGCATGTCTATGTGAAGAAAACGTTCACGGCGCTGTTT
GCATCAGGAATATGATCATTAAACATGGAGCGTAATGGTATTAATGATCA
ACTAGAAAAATGGTATGGAAGGGCGAGAGGGCGATCAACAAAGCAGCCCG
GGGCATAGTCTGGAAGCAGCAGGAATTGGAAGGGAAAAGGAAGCTGCACA
ATGAAGGGATATCGTGAGCGGAGTGGCTCACGAGAGTATCAACAGACTGG
CGAAAGCAAGCAATTGCCAACGCCGGCTATTAGGCCATAAGATGGCCTGT
TGTGAGTCCCAGTTGCACGTATCCCCATATGACTGCTCTGTCGCTGACTT
GAAAAAAAATAGGGAGGATAAAGGAGAAAGAAAGTGAGACAACCCGTGAG
GGACTTGGGGTAGTAGGAGAACACATGGGCAACCGGGCAATACACGCGAT
GTGAGACGAGTTCAACGGCGAATGGAAAATCTTGAAAAACAAAATAAAAT
AACTGCCCTCCATACGGGTATCAAATTCAAGCAGTTGTACGGAGGCTAGC
TAGAGTTGTGAAGTCGGTAATCCCGCTGTATAGTAATACGAGTCGCATCT
AAATACTCCGAAGCTGCTGCGAACCCGGAGAATCGAGATGTGCTGGAAAG
CTTCTAGCGAGCGGCTAAATTAGCATGAAAGGCTATGAGAAATTCTGGAG
ACGGCTTGTTGAATCATGGCGTTCCATTCTTCGACAAGCAAAGCGTTCCG
TCGCAGTAGCAGGCACTCATTCCCGAAAAAACTCGGAGATTCCTAAGTAG
CGATGGAACCGGAATAATATAATAGGCAATACATTGAGTTGCCTCGACGG
TTGCAATGCAGGGGTACTGAGCTTGGACATAACTGTTCCGTACCCCACCT
CTTCTCAACCTTTGGCGTTTCCCTGATTCAGCGTACCCGTACAAGTCGTA
ATCACTATTAACCCAGACTGACCGGACGTGTTTTGCCCTTCATTTGGAGA
AATAATGTCATTGCGATGTGTAATTTGCCTGCTTGACCGACTGGGGCTGT
TCGAAGCCCGAATGTAGGATTGTTATCCGAACTCTGCTCGTAGAGGCATG
TTGTGAATCTGTGTCGGGCAGGACACGCCTCGAAGGTTCACGGCAAGGGA
AACCACCGATAGCAGTGTCTAGTAGCAACCTGTAAAGCCGCAATGCAGCA
TCACTGGAAAATACAAACCAATGGCTAAAAGTACATAAGTTAATGCCTAA
AGAAGTCATATACCAGCGGCTAATAATTGTACAATCAAGTGGCTAAACGT
ACCGTAATTTGCCAACGGCTTGTGGGGTTGCAGAAGCAACGGCAAAGCCC
CACTTCCCCACGTTTGTTTCTTCACTCAGTCCAATCTCAGCTGGTGATCC
CCCAATTGGGTCGCTTGTTTGTTCCGGTGAAGTGAAAGAAGACAGAGGTA
AGAATGTCTGACTCGGAGCGTTTTGCATACAACCAAGGGCAGTGATGGAA
GACAGTGAAATGTTGACATTCAAGGAGTATTTAGCCAGGGATGCTTGAGT
GTATCGTGTAAGGAGGTTTGTCTGCCGATACGACGAATACTGTATAGTCA
CTTCTGATGAAGTGGTCCATATTGAAATGTAAAGTCGGCACTGAACAGGC
AAAAGATTGAGTTGAAACTGCCTAAGATCTCGGGCCCTCGGGCCTTCGGC
CTTTGGGTGTACATGTTTGTGCTCCGGGCAAATGCAAAGTGTGGTAGGAT
CGAACACACTGCTGCCTTTACCAAGCAGCTGAGGGTATGTGATAGGCAAA
TGTTCAGGGGCCACTGCATGGTTTCGAATAGAAAGAGAAGCTTAGCCAAG
AACAATAGCCGATAAAGATAGCCTCATTAAACGGAATGAGCTAGTAGGCA
AAGTCAGCGAATGTGTATATATAAAGGTTCGAGGTCCGTGCCTCCCTCAT
GCTCTCCCCATCTACTCATCAACTCAGATCCTCCAGGAGACTTGTACACC
ATCTTTTGAGGCACAGAAACCCAATAGTCAACCGCGGACTGCGCATCATG
TATCGGAAGTTGGCCGTCATCTCGGCCTTCTTGGCCACACCTCGTGCTAG
ACTAGGCGCGCCAGGAAGCCCGGAAGGTAAGTGGATTCTTCGCCGTGGCT
GGAGCAACCGGTGGATTCCAGCGTCTCCGACTTGGACTGAGCAATTCAGC
GTCACGGATTCACGATAGACAGCTCAGACCGCTCCACGGCTGGCGGCATT
ATTGGTTAACCCGGAAACTCAGTCTCCTTGGCCCCGTCCCGAAGGGACCC
GACTTACCAGGCTGGGAAAGCCAGGGATAGAATACACTGTACGGGCTTCG
TACGGGAGGTTCGGCGTAGGGTTGTTCCCAAGTTTTACACACCCCCCAAG
ACAGCTAGCGCACGAAAGACGCGGAGGGTTTGGTGAAAAAAGGGCGAAAA
TTAAGCGGGAGACGTATTTAGGTGCTAGGGCCGGTTTCCTCCCCATTTTT
CTTCGGTTCCCTTTCTCTCCTGGAAGACTTTCTCTCTCTCTCTTCTTCTC
TTCTTCCATCCTCAGTCCATCTTCCTTTCCCATCATCCATCTCCTCACCT
CCATCTCAACTCCATCACATCACAATCGATATGAAAAAGCCTGAACTCAC
CGCGACGTCTGTCGAGAAGTTTCTGATCGAAAAGTTCGACAGCGTCTCCG
ACCTGATGCAGCTCTCGGAGGGCGAAGAATCTCGTGCTTTCAGCTTCGAT
GTAGGAGGGCGTGGATATGTCCTGCGGGTAAATAGCTGCGCCGATGGTTT
CTACAAAGATCGTTATGTTTATCGGCACTTTGCATCGGCCGCGCTCCCGA
TTCCGGAAGTGCTTGACATTGGGGAATTCAGCGAGAGCCTGACCTATTGC
ATCTCCCGCCGTGCACAGGGTGTCACGTTGCAAGACCTGCCTGAAACCGA
ACTGCCCGCTGTTCTGCAGCCGGTCGCGGAGGCCATGGATGCGATCGCTG
CGGCCGATCTTAGCCAGACGAGCGGGTTCGGCCCATTCGGACCGCAAGGA
ATCGGTCAATACACTACATGGCGTGATTTCATATGCGCGATTGCTGATCC
CCATGTGTATCACTGGCAAACTGTGATGGACGACACCGTCAGTGCGTCCG
TCGCGCAGGCTCTCGATGAGCTGATGCTTTGGGCCGAGGACTGCCCCGAA
GTCCGGCACCTCGTGCACGCGGATTTCGGCTCCAACAATGTCCTGACGGA
CAATGGCCGCATAACAGCGGTCATTGACTGGAGCGAGGCGATGTTCGGGG
ATTCCCAATACGAGGTCGCCAACATCTTCTTCTGGAGGCCGTGGTTGGCT
TGTATGGAGCAGCAGACGCGCTACTTCGAGCGGAGGCATCCGGAGCTTGC
AGGATCGCCGCGGCTCCGGGCGTATATGCTCCGCATTGGTCTTGACCAAC
TCTATCAGAGCTTGGTTGACGGCAATTTCGATGATGCAGCTTGGGCGCAG
GGTCGATGCGACGCAATCGTCCGATCCGGAGCCGGGACTGTCGGGCGTAC
ACAAATCGCCCGCAGAAGCGCGGCCGTCTGGACCGATGGCTGTGTAGAAG
TACTCGCCGATAGTGGAAACCGACGCCCCAGCACTCGTCCGAGGGCAAAG
GAATAGAGTAGATGCCGACCGGGATCCACTTAACGTTACTGAAATCATCA
AACAGCTTGACGAATCTGGATATAAGATCGTTGGTGTCGATGTCAGCTCC
GGAGTTGAGACAAATGGTGTTCAGGATCTCGATAAGATACGTTCATTTGT
CCAAGCAGCAAAGAGTGCCTTCTAGTGATTTAATAGCTCCATGTCAACAA
GAATAAAACGCGTTTCGGGTTTACCTCTTCCAGATACAGCTCATCTGCAA
TGCATTAATGCATTGGACCTCGCAACCCTAGTACGCCCTTCAGGCTCCGG
CGAAGCAGAAGAATAGCTTAGCAGAGTCTATTTTCATTTTCGGGAGACTA
GCATTCTGTAAACGGGCAGCAATCGCCCAGCAGTTAGTAGGGTCCCCTCT
ACCTCTCAGGGAGATGTAACAACGCCACCTTATGGGACTATCAAGCTGAC
GCTGGCTTCTGTGCAGACAAACTGCGCCCACGAGTTCTTCCCTGACGCCG
CTCTCGCGCAGGCAAGGGAACTCGATGAATACTACGCAAAGCACAAGAGA
CCCGTTGGTCCACTCCATGGCCTCCCCATCTCTCTCAAAGACCAGCTTCG
AGTCAAGGTACACCGTTGCCCCTAAGTCGTTAGATGTCCCTTTTTGTCAG
CTAACATATGCCACCAGGGCTACGAAACATCAATGGGCTACATCTCATGG
CTAAACAAGTACGACGAAGGGGACTCGGTTCTGACAACCATGCTCCGCAA
AGCCGGTGCCGTCTTCTACGTCAAGACCTCTGTCCCGCAGACCCTGATGG
TCTGCGAGACAGTCAACAACATCATCGGGCGCACCGTCAACCCACGCAAC
AAGAACTGGTCGTGCGGCGGCAGTTCTGGTGGTGAGGGTGCGATCGTTGG
GATTCGTGGTGGCGTCATCGGTGTAGGAACGGATATCGGTGGCTCGATTC
GAGTGCCGGCCGCGTTCAACTTCCTGTACGGTCTAAGGCCGAGTCATGGG
CGGCTGCCGTATGCAAAGATGGCGAACAGCATGGAGGGTCAGGAGACGGT
GCACAGCGTTGTCGGGCCGATTACGCACTCTGTTGAGGGTGAGTCCTTCG
CCTCTTCCTTCTTTTCCTGCTCTATACCAGGCCTCCACTGTCCTCCTTTC
TTGCTTTTTATACTATATACGAGACCGGCAGTCACTGATGAAGTATGTTA
GACCTCCGCCTCTTCACCAAATCCGTCCTCGGTCAGGAGCCATGGAAATA
CGACTCCAAGGTCATCCCCATGCCCTGGCGCCAGTCCGAGTCGGACATTA
TTGCCTCCAAGATCAAGAACGGCGGGCTCAATATCGGCTACTACAACTTC
GACGGCAATGTCCTTCCACACCCTCCTATCCTGCGCGGCGTGGAAACCAC
CGTCGCCGCACTCGCCAAAGCCGGTCACACCGTGACCCCGTGGACGCCAT
ACAAGCACGATTTCGGCCACGATCTCATCTCCCATATCTACGCGGCTGAC
GGCAGCGCCGACGTAATGCGCGATATCAGTGCATCCGGCGAGCCGGCGAT
TCCAAATATCAAAGACCTACTGAACCCGAACATCAAAGCTGTTAACATGA
ACGAGCTCTGGGACACGCATCTCCAGAAGTGGAATTACCAGATGGAGTAC
CTTGAGAAATGGCGGGAGGCTGAAGAAAAGGCCGGGAAGGAACTGGACGC
CATCATCGCGCCGATTACGCCTACCGCTGCGGTACGGCATGACCAGTTCC
GGTACTATGGGTATGCCTCTGTGATCAACCTGCTGGATTTCACGAGCGTG
GTTGTTCCGGTTACCTTTGCGGATAAGAACATCGATAAGAAGAATGAGAG
TTTCAAGGCGGTTAGTGAGCTTGATGCCCTCGTGCAGGAAGAGTATGATC
CGGAGGCGTACCATGGGGCACCGGTTGCAGTGCAGGTTATCGGACGGAGA
CTCAGTGAAGAGAGGACGTTGGCGATTGCAGAGGAAGTGGGGAAGTTGCT
GGGAAATGTGGTGACTCCATAGCTAATAAGTGTCAGATAGCAATTTGCAC
AAGAAATCAATACCAGCAACTGTAAATAAGCGCTGAAGTGACCATGCCAT
GCTACGAAAGAGCAGAAAAAAACCTGCCGTAGAACCGAAGAGATATGACA
CGCTTCCATCTCTCAAAGGAAGAATCCCTTCAGGGTTGCGTTTCCAGTAG
TGATTTTACCGCTGATGAAATGACTGGACTCCCTCCTCCTGCTCTTATAC
GAAAAATTGCCTGACTCTGCAAAGGTTGTTTGTCTTGGAAGATGATGTGC
CCCCCCATCGCTCTTATCTCATACCCCGCCATCTTTCTAGATTCTCATCT
TCAACAAGAGGGGCAATCCATGATCTGCGATCCAGATGTGCTTCTGGCCT
CATACTCTGCCTTCAGGTTGATGTTCACTTAATTGGTGACGAATTCAGCT
GATTTGCTGCAGTATGCTTTGTGTTGGTTCTTTCCAGGCTTGTGCCAGCC
ATGAGCGCTTTGAGAGCATGTTGTCACTTATAAACTCGAGTAACGGCCAC
ATATTGTTCACTACTTGAATCACATACCTAATTTTGATAGAATTGACATG
TTTAAAGAGCTGAGGTAGCTTTAATGCCTCTGAAGTATTGTGACACAGCT
TCTCACAGAGTGAGAATGAAAAGTTGGACTCCCCCTAATGAAGTAAAAGT
TTCGTCTCTGAACGGTGAAGAGCATAGATCCGGCATCAACTACCTGGCTA
GACTACGACGTCAATTCTGCGGCCTTTTGACCTTTATATATGTCCATTAA
TGCAATAGATTCTTTTTTTTTTTTTTTTTTTTTTTTTTTTTTTTTTTTTT
TTTGCCCAATTTCGCAGATCAAAGTGGACGTTATAGCATCATAACTAAGC
TCAGTTGCTGAGGGAAGCCGTCTACTACCTTAGCCCATCCATCCAGCTCC
ATACCTTGATACTTTAGACGTGAAGCAATTCACACTGTACGTCTCGCAGC
TCTCCTTCCCGCTCTTGCTTCCCCACTGGGGTCCATGGTGCGTGTATCGT
CCCCTCCACAATTCTATGCCATGGTACCTCCAGCTTATCAATGCCCCGCT
AACAAGTCGCCTCTTTGCCTTGATAGCTTATCGATAAAACTTTTTTTCCG
CCAGAAAGGCTCCGCCCACAGACAAGAAAAAAAATTCACCGCCTAGCCTT
TGGCCCCGGCATTTGGCTAAACCTCGAGCCTCTCTCCCGTCTTGGGGTAT
CAGGAAGAAAAGAAAAAAATCCATCGCCAAGGGCTGTTTTGGCATCACCA
CCCGAAAACAGCACTTCCTCGATCAAAAGTTGCCCGCCATGAAGACCACG
TGGAAGGACATCCCTCCGGTGCCTACGCACCAGGAGTTTCTGGACATTGT
GCTGAGCAGGACCCAGCGCAAACTGCCCACTCAGATCCGTGCCGGCTTCA
AGATTAGCAGAATTCGAGGTACGTCGCATTGCCCATCGCAGGATGTCTCA
TTATCGGGGTCCTTGGAGAACGATCATGATTGCATGGCGATGCTAACACA
TAGACAGCCTTCTACACTCGAAAGGTCAAGTTCACCCAGGAGACGTTTTC
CGAAAAGTTCGCCTCCATCCTCGACAGCTTCCCTCGCCTCCAGGACATCC
ACCCCTTCCACAAGGACCTTCTCAACACCCTCTACGATGCCGACCACTTC
AAGATTGCCCTTGGCCAGATGTCCACTGCCAAGCACCTGGTCGAGACCAT
CTCGCGCGACTACGTCCGTCTCTTGAAATACGCCCAGTCGCTCTACCAGT
GCAAGCAGCTCAAGCGGGCCGCTCTCGGTCGCATGGCCACGCTGGTCAAG
CGCCTCAAGGACCCCCTGCTGTACCTGGACCAGGTCCGCCAGCATCTCGG
CCGTCTTCCCTCCATCGACCCCAACACCAGGACCCTGCTCATCTGCGGTT
ACCCCAATGTTGGCAAGTCCAGCTTCCTGCGAAGTATCACCCGCGCCGAT
GTGGACGTCCAGCCCTATGCTTTCACCACCAAGAGTCTGTTTGTCGGCCA
CTTTGACTACAAGTACCTGCGATTCCAGGCCATTGATACCCCCGGTATTC
TGGACCACCCTCTTGAGGAGATGAACACTATCGAAATGCAGAGGTATGTG
GCGCGGCTA.

Creation of the Archy3 strain from the Archy2 T. reesei strain. The Archy 2 strain was transformed with a vector to integrate at the same pyr2 locus and replace the hygromycin resistance gene with the coding region of the pyr2 gene. The hygromycin deletion cassette is shown in FIG. 4. This re-introduction of the pyr2 gene back into the pyr2 locus placed it between the T. reesei cbh1 promoter and the partial amdS selectable marker. This strain could be selected for uridine prototrophy and sensitivity to hygromycin. The nucleotide sequence of the hygR knockout cassette is 9088 base pairs in length: bases 1-1994 correspond to the pyr2 3′ homologous region; bases 1995-3497 correspond to the T. reesei cbh1 promoter; bases 3564-5137 correspond to the pyr2 selectable marker; bases 5280-7270 correspond to the A. nidulans amdS 3′ partial marker; bases 7271-9088 correspond to the pyr2 5′ homologous region. The nucleotide sequence of the hygR knockout cassette is provided as SEQ ID NO: 40:

ATCACGCCCTCGCATAAAAGACCCTCAAGAGTCCATGTGCCCTATCTGCC
TGATCTTCCTAACCCTTATTTAACATTGGCCCTATCACAACCTAGTTCTT
CTTCAGCCTGCTTTGTCAACACTTGTCACGGTTCAACTCAACGTAATCAG
CAGGTAGCAGGACGAGGATAGGGAGAGAAACGAAGAGAAGAAGAGGAGAG
AGGAAGAAAAAAAAAAGAAAAGAAAGAAAAAGGGAAAAGGAAAGAAGGAG
GAAAAGAGAAGAAAGTCAGATGAAGAAGCAAGAAGACGCCATGGTAGCCA
CCGCTTGTCAGGGCTCCTTAGCAACGAACAACTCTAGCTTGGGGACTTGT
CGATGTGTCGTTTCCTTCCTACCCATCAGCACCAACGATGAGTTCGATAT
AGACGAGGACCTCATGGAAGTAGAGACCATTGGGTTCGACAGGATCTCTC
AGTTTCACTTCTATGAGGTCTGTCGCTCGGATGACTTTTTGAGGAGCTTC
CCCTTCTGCTTCAACCCCAAACTCTCTTTCCTGAAACCGCAGCACGTTGG
CACGGCCGTGTTGCTGGAGCAGTTTGCTTTCGAGCACTCTCAGCGTGGTT
TCAGCAGCCCACTGGTGAGTGGCCTCCTTTGACGTCCACACCTTGCTCCT
GTCGCATGCGTATCTGGTGGGAACGACTGCTCCAAGGAGGATTGCTAACG
AGGTTGTAGGCCGAATATCGCATCAGATTCTCCGGTAACCTTAGCTACGG
CCTCTTCAACATCTGTGACATGACGGAGCGCAAGTACTGGTGGTTGGCGA
CCAAGATGCGCGGCTGGAACATCGACGGCTGCCCCGAAGACGTCAGGAGA
CTCATGTTTGTTCACATCATCGCCACCCTGGGATGCAGCCCCGTCGTGAC
GGATGAAGACATGGACTACCCCAAGAACTGGGCGGCAATTCTCCACGGTA
GAGACAGATATCCGAGTGAACCTGTGGGCCACCGGCCTCATGGGCGCACC
ATCTGCCTCCACTCGGTGGCCGTCTGCCCTCGTCTCCAGGGCTTGGGTCT
CGGTACTGCGACTCTGAAGTCGTATGTGCAGCGCATGAACAGCCTCGGCG
CCGCGGACCGTGTTGCTCTCGTTTGCCGCAAGCCCGAGACGAGATTTTTT
GAAAGATGCGGCTTCAGGAACAGCGGCCGGAGTAGTATCAAGACTCTGGT
CGGCGAATACTACAACATGGTGTGTGCTTCCACATCGACTTGGCCAGACT
CTATACGATTTTCAAACCTCGCTATACGTCATATTGACTTGTTTCTTTAG
GTCTTCGATTTGCCCGGGCCCAAAGACTTTATCGACTGGAATAGCATTGC
CGACGCTGCCAAGAAGATGTGAACCATTTGACTGATACGATGTGTGCTAC
GCATGTCGACCTTCTTTGTTTGTTTCTTTGGCGGCTCTTTGTATACCTTG
GGACACGGCAGACGCATGTCTATGTGAAGAAAACGTTCACGGCGCTGTTT
GCATCAGGAATATGATCATTAAACATGGAGCGTAATGGTATTAATGATCA
ACTAGAAAAATGGTATGGAAGGGCGAGAGGGCGATCAACAAAGCAGCCCG
GGGCATAGTCTGGAAGCAGCAGGAATTGGAAGGGAAAAGGAAGCTGCACA
ATGAAGGGATATCGTGAGCGGAGTGGCTCACGAGAGTATCAACAGACTGG
CGAAAGCAAGCAATTGCCAACGCCGGCTATTAGGCCATAAGATGGCCTGT
TGTGAGTCCCAGTTGCACGTATCCCCATATGACTGCTCTGTCGCTGACTT
GAAAAAAAATAGGGAGGATAAAGGAGAAAGAAAGTGAGACAACCCGTGAG
GGACTTGGGGTAGTAGGAGAACACATGGGCAACCGGGCAATACACGCGAT
GTGAGACGAGTTCAACGGCGAATGGAAAATCTTGAAAAACAAAATAAAAT
AACTGCCCTCCATACGGGTATCAAATTCAAGCAGTTGTACGGAGGCTAGA
TAGAGTTGTGAAGTCGGTAATCCCGCTGTATAGTAATACGAGTCGCATCT
AAATACTCCGAAGCTGCTGCGAACCCGGAGAATCGAGATGTGCTGGAAAG
CTTCTAGCGAGCGGCTAAATTAGCATGAAAGGCTATGAGAAATTCTGGAG
ACGGCTTGTTGAATCATGGCGTTCCATTCTTCGACAAGCAAAGCGTTCCG
TCGCAGTAGCAGGCACTCATTCCCGAAAAAACTCGGAGATTCCTAAGTAG
CGATGGAACCGGAATAATATAATAGGCAATACATTGAGTTGCCTCGACGG
TTGCAATGCAGGGGTACTGAGCTTGGACATAACTGTTCCGTACCCCACCT
CTTCTCAACCTTTGGCGTTTCCCTGATTCAGCGTACCCGTACAAGTCGTA
ATCACTATTAACCCAGACTGACCGGACGTGTTTTGCCCTTCATTTGGAGA
AATAATGTCATTGCGATGTGTAATTTGCCTGCTTGACCGACTGGGGCTGT
TCGAAGCCCGAATGTAGGATTGTTATCCGAACTCTGCTCGTAGAGGCATG
TTGTGAATCTGTGTCGGGCAGGACACGCCTCGAAGGTTCACGGCAAGGGA
AACCACCGATAGCAGTGTCTAGTAGCAACCTGTAAAGCCGCAATGCAGCA
TCACTGGAAAATACAAACCAATGGCTAAAAGTACATAAGTTAATGCCTAA
AGAAGTCATATACCAGCGGCTAATAATTGTACAATCAAGTGGCTAAACGT
ACCGTAATTTGCCAACGGCTTGTGGGGTTGCAGAAGCAACGGCAAAGCCC
CACTTCCCCACGTTTGTTTCTTCACTCAGTCCAATCTCAGCTGGTGATCC
CCCAATTGGGTCGCTTGTTTGTTCCGGTGAAGTGAAAGAAGACAGAGGTA
AGAATGTCTGACTCGGAGCGTTTTGCATACAACCAAGGGCAGTGATGGAA
GACAGTGAAATGTTGACATTCAAGGAGTATTTAGCCAGGGATGCTTGAGT
GTATCGTGTAAGGAGGTTTGTCTGCCGATACGACGAATACTGTATAGTCA
CTTCTGATGAAGTGGTCCATATTGAAATGTAAAGTCGGCACTGAACAGGC
AAAAGATTGAGTTGAAACTGCCTAAGATCTCGGGCCCTCGGGCCTTCGGC
CTTTGGGTGTACATGTTTGTGCTCCGGGCAAATGCAAAGTGTGGTAGGAT
CGAACACACTGCTGCCTTTACCAAGCAGCTGAGGGTATGTGATAGGCAAA
TGTTCAGGGGCCACTGCATGGTTTCGAATAGAAAGAGAAGCTTAGCCAAG
AACAATAGCCGATAAAGATAGCCTCATTAAACGGAATGAGCTAGTAGGCA
AAGTCAGCGAATGTGTATATATAAAGGTTCGAGGTCCGTGCCTCCCTCAT
GCTCTCCCCATCTACTCATCAACTCAGATCCTCCAGGAGACTTGTACACC
ATCTTTTGAGGCACAGAAACCCAATAGTCAACCGCGGACTGCGCATCATG
TATCGGAAGTTGGCCGTCATCTCGGCCTTCTTGGCCACACCTCGTGCTAG
ACTAGGCGCGTCAATATGTGGCCGTTACTCGAGTTTATAAGTGACAACAT
GCTCTCAAAGCGCTCATGGCTGGCACAAGCCTGGAAAGAACCAACACAAA
GCATACTGCAGCAAATCAGCTGAATTCGTCACCAATTAAGTGAACATCAA
CCTGAAGGCAGAGTATGAGGCCAGAAGCACATCTGGATCGCAGATCATGG
ATTGCCCCTCTTGTTGAAGATGAGAATCTAGAAAGATGGCGGGGTATGAG
ATAAGAGCGATGGGGGGGCACATCATCTTCCAAGACAAACAACCTTTGCA
GAGTCAGGCAATTTTTCGTATAAGAGCAGGAGGAGGGAGTCCAGTCATTT
CATCAGCGGTAAAATCACTCTAGACAATCTTCAAGATGAGTTCTGCCTTG
GGTGACTTATAGCCATCATCATACCTAGACAGAAGCTTGTGGGATACTAA
GACCAACGTACAAGCTCGCACTGTACGCTTTGACTTCCATGTGAAAACTC
GATACGGCGCGCCTCTAAATTTTATAGCTCAACCACTCCAATCCAACCTC
TGCATCCCTCTCACTCGTCCTGATCTACTGTTCAAATCAGAGAATAAGGA
CACTATCCAAATCCAACAGAATGGCTACCACCTCCCAGCTGCCTGCCTAC
AAGCAGGACTTCCTCAAATCCGCCATCGACGGCGGCGTCCTCAAGTTTGG
CAGCTTCGAGCTCAAGTCCAAGCGGATATCCCCCTACTTCTTCAACGCGG
GCGAATTCCACACGGCGCGCCTCGCCGGCGCCATCGCCTCCGCCTTTGCA
AAGACCATCATCGAGGCCCAGGAGAAGGCCGGCCTAGAGTTCGACATCGT
CTTCGGCCCGGCCTACAAGGGCATCCCGCTGTGCTCCGCCATCACCATCA
AGCTCGGCGAGCTGGCGCCCCAGAACCTGGACCGCGTCTCCTACTCGTTT
GACCGCAAGGAGGCCAAGGACCACGGCGAGGGCGGCAACATCGTCGGCGC
TTCGCTCAAGGGCAAGAGGGTCCTGATTGTCGACGACGTCATCACCGCCG
GCACCGCCAAGAGGGACGCCATTGAGAAGATCACCAAGGAGGGCGGCATC
GTCGCCGGCATCGTCGTGGCCCTGGACCGCATGGAGAAGCTCCCCGCTGC
GGATGGCGACGACTCCAAGCCTGGACCGAGTGCCATTGGCGAGCTGAGGA
AGGAGTACGGCATCCCCATCTTTGCCATCCTCACTCTGGATGACATTATC
GATGGCATGAAGGGCTTTGCTACCCCTGAGGATATCAAGAACACGGAGGA
TTACCGTGCCAAGTACAAGGCGACTGACTGATTGAGGCGTTCAATGTCAG
AAGGGAGAGAAAGACTGAAAAGGTGGAAAGAAGAGGCAAATTGTTGTTAT
TATTATTATTCTATCTCGAATCTTCTAGATCTTGTCGTAAATAAACAAGC
GTAACTAGCTAGCCTCCGTACAACTGCTTGAATTTGATACCCGTATGGAG
GGCAGTTATTTTATTTTGTTTTTCAAGATTTTCCATTCGCCGTTGAACTC
GTCTCACATCGCGTGTATTGCCCGGTTGCCCATGTGTACGCGTTTCGGGT
TTACCTCTTCCAGATACAGCTCATCTGCAATGCATTAATGCATTGGACCT
CGCAACCCTAGTACGCCCTTCAGGCTCCGGCGAAGCAGAAGAATAGCTTA
GCAGAGTCTATTTTCATTTTCGGGAGACTAGCATTCTGTAAACGGGCAGC
AATCGCCCAGCAGTTAGTAGGGTCCCCTCTACCTCTCAGGGAGATGTAAC
AACGCCACCTTATGGGACTATCAAGCTGACGCTGGCTTCTGTGCAGACAA
ACTGCGCCCACGAGTTCTTCCCTGACGCCGCTCTCGCGCAGGCAAGGGAA
CTCGATGAATACTACGCAAAGCACAAGAGACCCGTTGGTCCACTCCATGG
CCTCCCCATCTCTCTCAAAGACCAGCTTCGAGTCAAGGTACACCGTTGCC
CCTAAGTCGTTAGATGTCCCTTTTTGTCAGCTAACATATGCCACCAGGGC
TACGAAACATCAATGGGCTACATCTCATGGCTAAACAAGTACGACGAAGG
GGACTCGGTTCTGACAACCATGCTCCGCAAAGCCGGTGCCGTCTTCTACG
TCAAGACCTCTGTCCCGCAGACCCTGATGGTCTGCGAGACAGTCAACAAC
ATCATCGGGCGCACCGTCAACCCACGCAACAAGAACTGGTCGTGCGGCGG
CAGTTCTGGTGGTGAGGGTGCGATCGTTGGGATTCGTGGTGGCGTCATCG
GTGTAGGAACGGATATCGGTGGCTCGATTCGAGTGCCGGCCGCGTTCAAC
TTCCTGTACGGTCTAAGGCCGAGTCATGGGCGGCTGCCGTATGCAAAGAT
GGCGAACAGCATGGAGGGTCAGGAGACGGTGCACAGCGTTGTCGGGCCGA
TTACGCACTCTGTTGAGGGTGAGTCCTTCGCCTCTTCCTTCTTTTCCTGC
TCTATACCAGGCCTCCACTGTCCTCCTTTCTTGCTTTTTATACTATATAC
GAGACCGGCAGTCACTGATGAAGTATGTTAGACCTCCGCCTCTTCACCAA
ATCCGTCCTCGGTCAGGAGCCATGGAAATACGACTCCAAGGTCATCCCCA
TGCCCTGGCGCCAGTCCGAGTCGGACATTATTGCCTCCAAGATCAAGAAC
GGCGGGCTCAATATCGGCTACTACAACTTCGACGGCAATGTCCTTCCACA
CCCTCCTATCCTGCGCGGCGTGGAAACCACCGTCGCCGCACTCGCCAAAG
CCGGTCACACCGTGACCCCGTGGACGCCATACAAGCACGATTTCGGCCAC
GATCTCATCTCCCATATCTACGCGGCTGACGGCAGCGCCGACGTAATGCG
CGATATCAGTGCATCCGGCGAGCCGGCGATTCCAAATATCAAAGACCTAC
TGAACCCGAACATCAAAGCTGTTAACATGAACGAGCTCTGGGACACGCAT
CTCCAGAAGTGGAATTACCAGATGGAGTACCTTGAGAAATGGCGGGAGGC
TGAAGAAAAGGCCGGGAAGGAACTGGACGCCATCATCGCGCCGATTACGC
CTACCGCTGCGGTACGGCATGACCAGTTCCGGTACTATGGGTATGCCTCT
GTGATCAACCTGCTGGATTTCACGAGCGTGGTTGTTCCGGTTACCTTTGC
GGATAAGAACATCGATAAGAAGAATGAGAGTTTCAAGGCGGTTAGTGAGC
TTGATGCCCTCGTGCAGGAAGAGTATGATCCGGAGGCGTACCATGGGGCA
CCGGTTGCAGTGCAGGTTATCGGACGGAGACTCAGTGAAGAGAGGACGTT
GGCGATTGCAGAGGAAGTGGGGAAGTTGCTGGGAAATGTGGTGACTCCAT
AGCTAATAAGTGTCAGATAGCAATTTGCACAAGAAATCAATACCAGCAAC
TGTAAATAAGCGCTGAAGTGACCATGCCATGCTACGAAAGAGCAGAAAAA
AACCTGCCGTAGAACCGAAGAGATATGACACGCTTCCATCTCTCAAAGGA
AGAATCCCTTCAGGGTTGCGTTTCCAGTAGTGATTTTACCGCTGATGAAA
TGACTGGACTCCCTCCTCCTGCTCTTATACGAAAAATTGCCTGACTCTGC
AAAGGTTGTTTGTCTTGGAAGATGATGTGCCCCCCCATCGCTCTTATCTC
ATACCCCGCCATCTTTCTAGATTCTCATCTTCAACAAGAGGGGCAATCCA
TGATCTGCGATCCAGATGTGCTTCTGGCCTCATACTCTGCCTTCAGGTTG
ATGTTCACTTAATTGGTGACGAATTCAGCTGATTTGCTGCAGTATGCTTT
GTGTTGGTTCTTTCCAGGCTTGTGCCAGCCATGAGCGCTTTGAGAGCATG
TTGTCACTTATAAACTCGAGTAACGGCCACATATTGTTCACTACTTGAAT
CACATACCTAATTTTGATAGAATTGACATGTTTAAAGAGCTGAGGTAGCT
TTAATGCCTCTGAAGTATTGTGACACAGCTTCTCACAGAGTGAGAATGAA
AAGTTGGACTCCCCCTAATGAAGTAAAAGTTTCGTCTCTGAACGGTGAAG
AGCATAGATCCGGCATCAACTACCTGGCTAGACTACGACGTCAATTCTGC
GGCCTTTTGACCTTTATATATGTCCATTAATGCAATAGATTCTTTTTTTT
TTTTTTTTTTTTTTTTTTTTTTTTTTTTTTTTTGCCCAATTTCGCAGATC
AAAGTGGACGTTATAGCATCATAACTAAGCTCAGTTGCTGAGGGAAGCCG
TCTACTACCTTAGCCCATCCATCCAGCTCCATACCTTGATACTTTAGACG
TGAAGCAATTCACACTGTACGTCTCGCAGCTCTCCTTCCCGCTCTTGCTT
CCCCACTGGGGTCCATGGTGCGTGTATCGTCCCCTCCACAATTCTATGCC
ATGGTACCTCCAGCTTATCAATGCCCCGCTAACAAGTCGCCTCTTTGCCT
TGATAGCTTATCGATAAAACTTTTTTTCCGCCAGAAAGGCTCCGCCCACA
GACAAGAAAAAAAATTCACCGCCTAGCCTTTGGCCCCGGCATTTGGCTAA
ACCTCGAGCCTCTCTCCCGTCTTGGGGTATCAGGAAGAAAAGAAAAAAAT
CCATCGCCAAGGGCTGTTTTGGCATCACCACCCGAAAACAGCACTTCCTC
GATCAAAAGTTGCCCGCCATGAAGACCACGTGGAAGGACATCCCTCCGGT
GCCTACGCACCAGGAGTTTCTGGACATTGTGCTGAGCAGGACCCAGCGCA
AACTGCCCACTCAGATCCGTGCCGGCTTCAAGATTAGCAGAATTCGAGGT
ACGTCGCATTGCCCATCGCAGGATGTCTCATTATCGGGGTCCTTGGAGAA
CGATCATGATTGCATGGCGATGCTAACACATAGACAGCCTTCTACACTCG
AAAGGTCAAGTTCACCCAGGAGACGTTTTCCGAAAAGTTCGCCTCCATCC
TCGACAGCTTCCCTCGCCTCCAGGACATCCACCCCTTCCACAAGGACCTT
CTCAACACCCTCTACGATGCCGACCACTTCAAGATTGCCCTTGGCCAGAT
GTCCACTGCCAAGCACCTGGTCGAGACCATCTCGCGCGACTACGTCCGTC
TCTTGAAATACGCCCAGTCGCTCTACCAGTGCAAGCAGCTCAAGCGGGCC
GCTCTCGGTCGCATGGCCACGCTGGTCAAGCGCCTCAAGGACCCCCTGCT
GTACCTGGACCAGGTCCGCCAGCATCTCGGCCGTCTTCCCTCCATCGACC
CCAACACCAGGACCCTGCTCATCTGCGGTTACCCCAATGTTGGCAAGTCC
AGCTTCCTGCGAAGTATCACCCGCGCCGATGTGGACGTCCAGCCCTATGC
TTTCACCACCAAGAGTCTGTTTGTCGGCCACTTTGACTACAAGTACCTGC
GATTCCAGGCCATTGATACCCCCGGTATTCTGGACCACCCTCTTGAGGAG
ATGAACACTATCGAAATGCAGAGGTATGTGGCGCGGCT.

### Example 2

### Laccase Variants with Added Glycosylation Sites

Seven glycosylation sites were engineered on the surface of the Cerrena laccase D polypeptide. Briefly, seven pairs of oligonucleotides were prepared for use in use in standard techniques to introduce the following amino acid residue changes at the indicated positions, referring to SEQ ID NO: 11: NKD to NAT at residues 12 to 14 (variant mut1), GGT to NGT at residues 28 to 30 (variant mut2), NVI to NVT at residues 47 to 49 (variant mut3), QTV to NTT at residues 157 to 159 (variant mut4), NAV to NAT at residues 317 to 319 (variant mut5), NAQ to NAS at residues 362 to 364 (variant mut6), and SAS to NAS at residues 492 to 494 (variant mut7). The PCR-mediated mutagenesis reaction contained 2 μl of template plasmid DNA (i.e., pKB409, a pENTR plasmid that includes the nucleotide sequence encoding the signal sequence of the Trichoderma CBH1 gene and the mature Cerrena laccase D1 protein, without an amdS marker; 5 ng/μl), 5 μl of standard 10× buffer, 1.5 μl of 100 mM dNTPs, 1.25 μl of 100 ng/μl forward primer, 1.25 μl of 100 ng/μl reverse primer, and 1 μl of Pfu Ultra II polymerase (Stratagene, La Jolla, Calif., USA) in a 50 μl reaction volume. The PCR products were digested with the DpnI restriction enzyme (Roche) and 5 μl of each mixture containing nicked plasmid DNA was transformed into E. coli cells. DNA was prepared from each of the transformants and the engineered nucleotide changes were confirmed by DNA sequencing.

The mutated coding sequences were then cloned into expression plasmid pTrex3g using the gateway cloning method in a reaction containing 0.5 μl of plasmid DNA, 0.5 μl of pTrex3g, 3 μl of TE buffer, and 1 μl of LRII mixture (Invitrogen), which was reacted at room temperature for one hour and then transformed to E. coli cells. pTrex3g, described in U.S. Patent Pub. No. 20100304468, is based on the E. coli vector pSL1180 (Pharmacia Inc., Piscataway, N.J.), which is a pUC118 phagemid based vector (Brosius, J. (1989) DNA 8:759) with an extended multiple cloning site containing 64 hexamer restriction enzyme recognition sequences. The vector is designed as a Gateway destination vector (Hartley et al. (2000) Genome Research 10:1788-95) to allow insertion using Gateway technology (Invitrogen) of any desired open reading frame between the promoter and terminator regions of the T. reesei cbh1 gene. The vector also contains the Aspergillus nidulans amdS gene for use as a selective marker. DNA prepared from each of the transformants was subjected to nucleotide sequence analysis to confirm the engineered nucleotide changes.

Three to six 100 μl PCR replicates were performed using each of the seven variant coding sequences as templates. The resulting PCR fragments were used to transform the Archy3 strain of T. reesei. Briefly, frozen Archy3 strain protoplasts were thawed on ice and 100 μl portions were transferred to 15 ml tubes. 5-15 μl of each PCR fragment were separately added to the protoplasts, and the DNA and protoplast mixtures were left on ice for 20 minutes. 2 ml of FF4 [25% PEG 6000, 50 mM CaCl, and 10 mM Tris (pH 7.5)] buffer was then added to each tube of protoplasts, followed by incubation at room temperature for 5 minutes. 4 ml of FF3 [1.2 M sorbitol, 10 mM CaCl, and 10 mM Tris (pH 7.5)] were added and the entire volume was transferred to a new tube for equal distribution onto two petri dishes. 25 ml overlay [amdS-sorbitol-agarose (2%)+uridine (0.5 mg/ml)] were overlayed on each of the plates, which were then incubated for 5-6 days at 28° C. Detailed methods for using amdS marker system in the transformation of industrially important filamentous fungi are established in the art (e.g., in Aspergillus niger (see, e.g., Kelly and Hynes (1985) EMBO J. 4:475-79; Wang et al. (2008) Fungal Genet Biol. 45:17-27); in Penicillium chrysogenum(see, e.g., Beri and Turner (1987) Curr. Genet. 11:639-41); in Trichoderma reesei (see, e.g., Pentilla et al. (1987) Gene 61:155-64); in Aspergillus oryzae (see, e.g., Christensen et al. (1988) Bio/technology 6:1419-22); in Trichoderma harzianum (see, e.g., Pe'er et al. (1991) Soil Biol. Biochem. 23:1043-46); and in U.S. Pat. No. 6,548,285; each of which references is hereby incorporated by reference.

For each transformation, five colonies were selected and transferred to a conventional potato dextrose agar (PDA) plate contain 1.2 mg/ml 5-FOA and 0.5 mg/ml uridine. The Archy3 strain with integrated plasmid expressing wild type laccase was used as control. The plates were grown at 28° C. for 2 days and left at room temperature for one day to encourage sporulation. All five clones were transferred to a 96-well microtiter filter plate (MTP, Corning 3505) filled with 200 μl defined medium with glucose/sophorose (33.0 g/L PIPPS buffer; 9.0 g/L casamino acids; 5.0 g/L (NH₄)₂SO₄; 4.5 g/L KH₂PO₄; 1.0 g/L MgSO₄.7H₂O; 1.0 g/L CaCl₂; 26 ml/L 60% glucose/sophorose mixture; 2.5 ml/L 400X T. reesei trace elements: 175 g/L citric acid anhydrous; 200 g/L FeSO₄.7H₂O; 16 g/L ZnSO₄.7H₂O; 3.2 g/L CuSO₄.5H₂O; 1.4 g/L MnSO₄.H₂O; 0.8 g/L H₃BO₃; and 0.5 mg/ml uridine; pH 5.5). The MTP filter plate was grown at 28° C. with a constant oxygen supply and without shaking for 5 days. 10 μl of 5-days old liquid cultures were transferred to a new plate and 150 μl 100 mM NaOAc, pH 5, and 20 μl 4.5 mM ABTS were added.

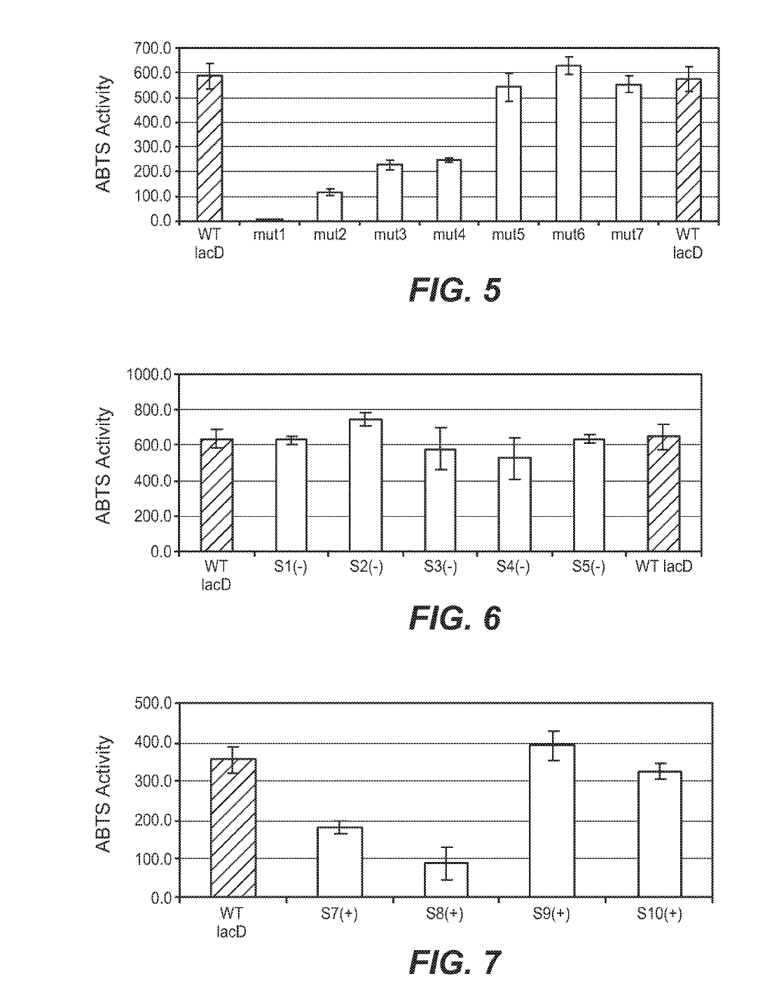

The OD₄₂₀ was measured using a Spectra Max spectrophotomoeter for 5 minutes at 20-second intervals. The laccase activity present in the liquid cultures containing filamentous fungi transformed with each of the seven glycosylation mutants is shown in the graph of FIG. 5. Error bars in this and other graphs indicate standard deviation. Mut6, which has the NAQ to NAS change, demonstrated a 7% average increase in laccase activity compared to wild-type based on the average laccase activity, although the error bars suggest that the difference may not be significant.

### Example 3

### Laccase Variants with Additional Positively or Negatively Charged Amino Acid Residues

Five positively or five negatively charged amino acid residues were introduced on the surface of the Cerrena laccase. Briefly, ten pairs of oligonucleotides (i.e., forward and reverse primers) were prepared to introduce the following amino acid residue changes at the indicated positions (referring to SEQ ID NO: 11): Q21E (variant S1), N130E (variant S2), T232E (variant S3), N335E (variant S4), Q479E (variant S5), Q21R (variant S6), N130R (variant S7), T232R (variant S8), N335R (variant S9), and Q479R (variant S10). PCR-mediated mutagenesis, E. coli transformation, and verification of the mutations, were performed as in Example 2.

The PCR fragments were used to transform the Archy3 strain of T. reesei as in Example 2. Transformants were selected and transferred to an amdS plate (supra) containing 1.2 mg/ml 5-FOA and 0.5 mg/ml of uridine and grown at 28° C. for 2 days. For each variant, four colonies were selected and transferred to a PDA plate containing 1.2 mg/ml 5-FOA and 0.5 mg/ml uridine. The Archy3 strain with integrated plasmid expressing wild type laccase was used as control. The plates were grown at 28° C. for 1 day and left at room temperature for 3 days to encourage sporulation. All clones were transferred to a 96-well microtiter filter plate (MTP, Corning 3505) filled with 200 μl NREL defined medium with glucose/sophomores and 0.5 mg/ml uridine. The MTP filter plate was grown at 28° C. with a constant oxygen supply and without shaking for 5 days. 10 μl of 5-days old liquid cultures were transferred to a new plate and 150 μl 100 mM NaOAc, pH 5, and 20 μl 4.5 mM ABTS were added.

The OD₄₂₀ was measured as in Example 2. The results are shown in FIG. 6 (variants S1 to S5, which include a neutral amino acid residue changed to a negatively charged residue) and FIG. 7 (variants S7 to S10, which include a neutral amino acid residue changed to a positively charged residue). Variant S2 demonstrated 17% increased laccase activity compared to wild-type. Variant S9 demonstrated a 10% increased laccase activity compared to wild-type, although this latter difference may not be significant.

### Example 4

### Site Evaluation Library #1 (SEL1) Variants

A non-conservative, hydrophobic amino acid residue (1265) located on the surface of Cerrena laccase was selected for further engineering. Briefly, a pair of complementary oligonucleotide primers overlapping the I265 codon were prepared and used to introduce amino acid residue changes at this position. PCR-mediated mutagenesis reaction was performed as in Example 2. The PCR products were digested with DpnI restriction enzyme for 2 hours at 37° C. and purified using a Qiagen column. The SEL library variants were then cloned to expression plasmid pTrex3g using the gateway cloning method in a reaction containing of 3 μl of PCR product, 0.5 μl of pTrex3g, 0.5 μl of TE buffer, and 1 μl of LRII mixture (Invitrogen), which were incubated at room temperature for one hour. The mixture was transformed into E. coli cells.

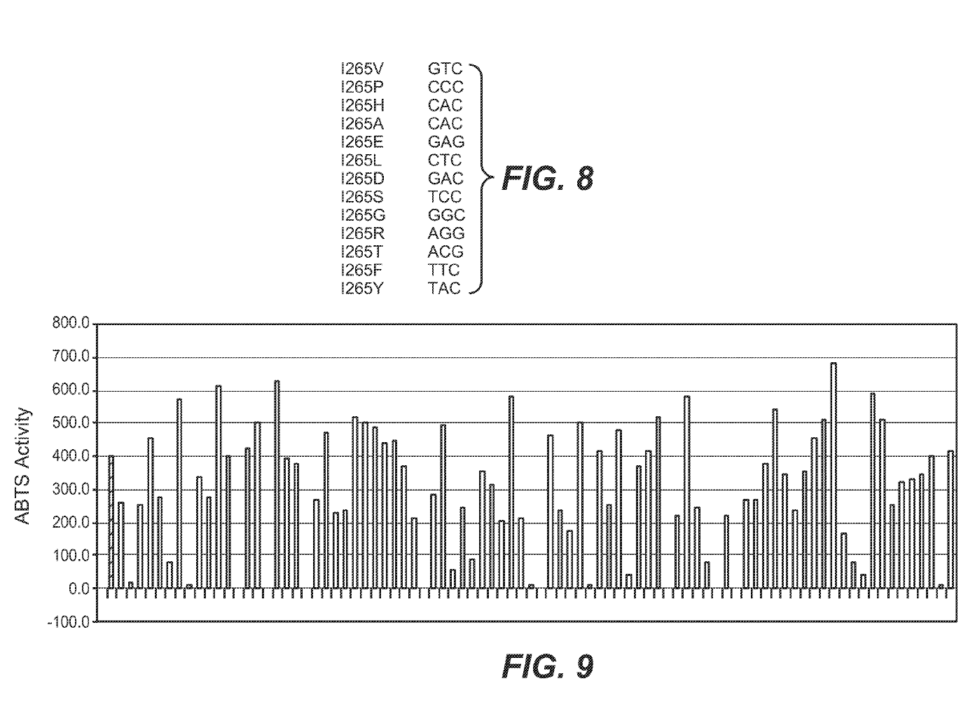

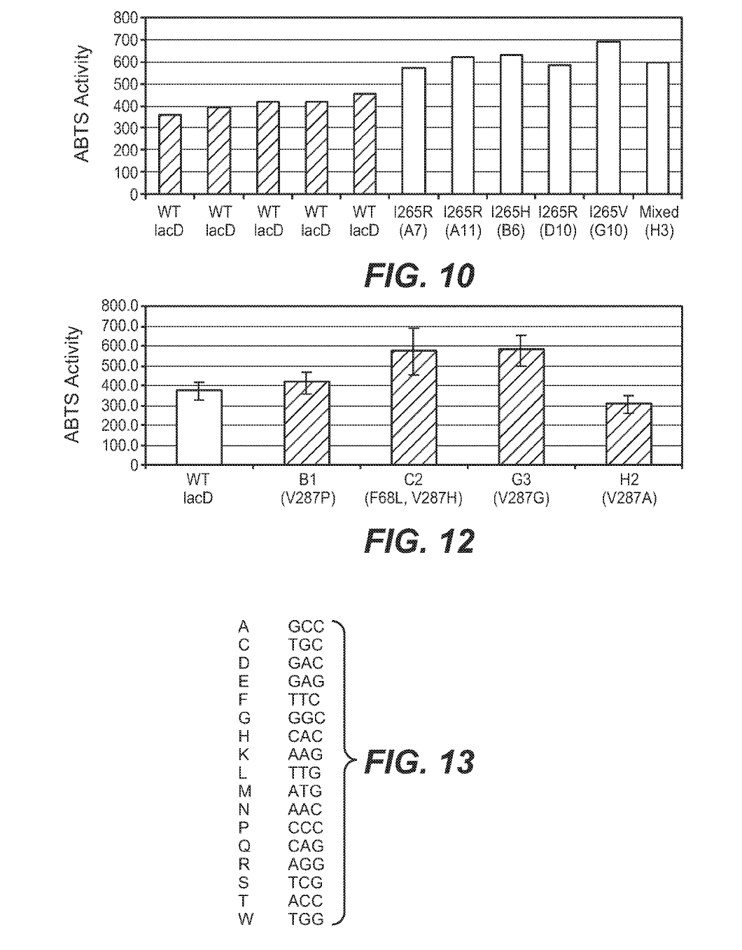

DNA was prepared from 28 clones and subjected to DNA sequence analysis. A total of 13 variants were obtained. The codons and corresponding amino acid residues at position 265 are listed in FIG. 8. 5 μl DNA of each of the 13 variants was pooled and used as DNA template for PCR fragment amplification. Ten tubes of 100-0 PCR mixes were prepared and used to transform the Archy3 strain of T. reesei as in Example 2, except that 10 mls of overlays containing 1.2 mg/ml 5-FOA and 0.5 mg/ml uridine were added after 24 hours incubation at 28° C. Transformants were selected and transferred to two 48-well MTPs filled with 1 ml of PDA containing 1.2 mg/ml 5-FOA and 0.5 mg/ml uridine. The MTPs were grown at 28° C. for 2 days and left at room temperature for 2 day to promote sporulation. All clones were individually transferred to a 96-well filter plate and incubated for 5 days. The ABTS assay was performed as in Example 2. FIG. 9 shows the ABTS activity assay result for all 88 transformants screened. The identity of the variant laccase sequences were unknown at the time of screening, therefore the X-axis has no labels. Six clones showing higher ABTS activity were selected for further study, hence corresponding mycellia from the filter plate were grown in YEG for genomic DNA extraction. A 600 bp fragment was amplified using two primers flanking the codon corresponding to amino acid position 265. The PCR fragments were sequenced to identify the mutations present. FIG. 10 shows the variants that produced the highest laccase expression or activity, i.e., I265R, I265H, and I265V.

### Example 5

### Site Evaluation Library #2 (SEL2) Variants

A non-conservative, hydrophobic amino acid residue (V287) located on the surface of Cerrena laccase was selected for further engineering. Briefly, pairs of complementary primers overlapping the V287 codon were prepared and used to introduce amino acid residue changes. The SEL2 library variants were generated in same way as the SEL1 variants described in Example 4, except that all E. coli transformants were pooled and plasmids were extracted from pooled E. coli cells and used as mixed DNA template for PCR fragment amplification.

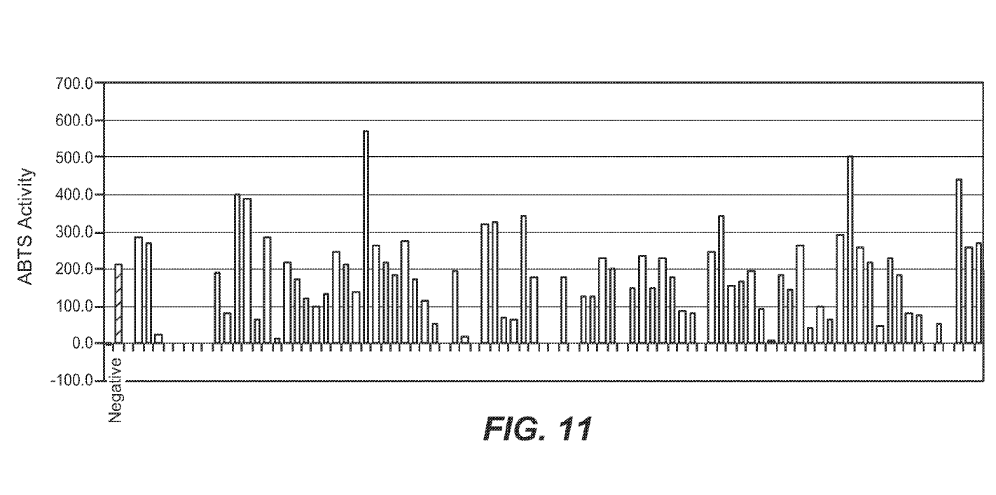

Ten tubes of 100 μl PCR reactions were prepared, and the PCR fragments were transformed into the Archy3 strain of T. reesei as in Example 4. Transformants were selected and transferred to 96-well MTPs filled with 0.2 ml of PDA containing 1.2 mg/ml 5-FOA and 0.5 mg/ml of uridine. The MTPs were incubated at 28° C. for 2 days and left at room temperature for 3 days to promote sporulation. All clones were transferred to a 96-well filter plate using a metal replicator for 96 well plates (Boekel). The filter plate was incubated for 5 days and the ABTS assay was performed as in Example 2. FIG. 11 shows the ABTS activity assay result for all 88 transformants screened. As in Example 4, the identity of the variant laccase sequences were unknown at the time of screening, therefore the X-axis has no labels.

Genomic DNA was prepared using 5-day old mycellium from the filter plate, and subjected to DNA sequence analysis. A total of 14 variants were identified, i.e., V287A, V287D, V287E, V287F, V287G, V287H, V287L, V287N, V287P, V287Q, V287, V287S, V287T, and V287W. FIG. 12 shows data obtained from the three best variants, i.e., B1, which includes the V287P mutation, C2, which includes the V287H mutation as well as another mutation (F68L), presumably resulting from a PCR error, and G3, which includes the V287G mutation.

### Example 6

### Site Evaluation Library #3 (SEL3) Variants

A non-conservative, hydrophobic amino acid residue (V319) located on the surface of Cerrena laccase was selected for further engineering. Briefly, pairs of complementary primers overlapping the V319 codon were prepared and used to introduce amino acid residue changes. The SEL3 library variants were generated in same way as the SEL1 variants in Example 4, except that all E. coli colonies were subjected to DNA sequence analysis. E. coli cultures of 17 variants were pooled and plasmid DNA was extracted. FIG. 13 lists the 17 variants identified. The DNA was then used as template for PCR, and the PCR fragments were transformed into the Archy3 strain of T. reesei as in Example 4.

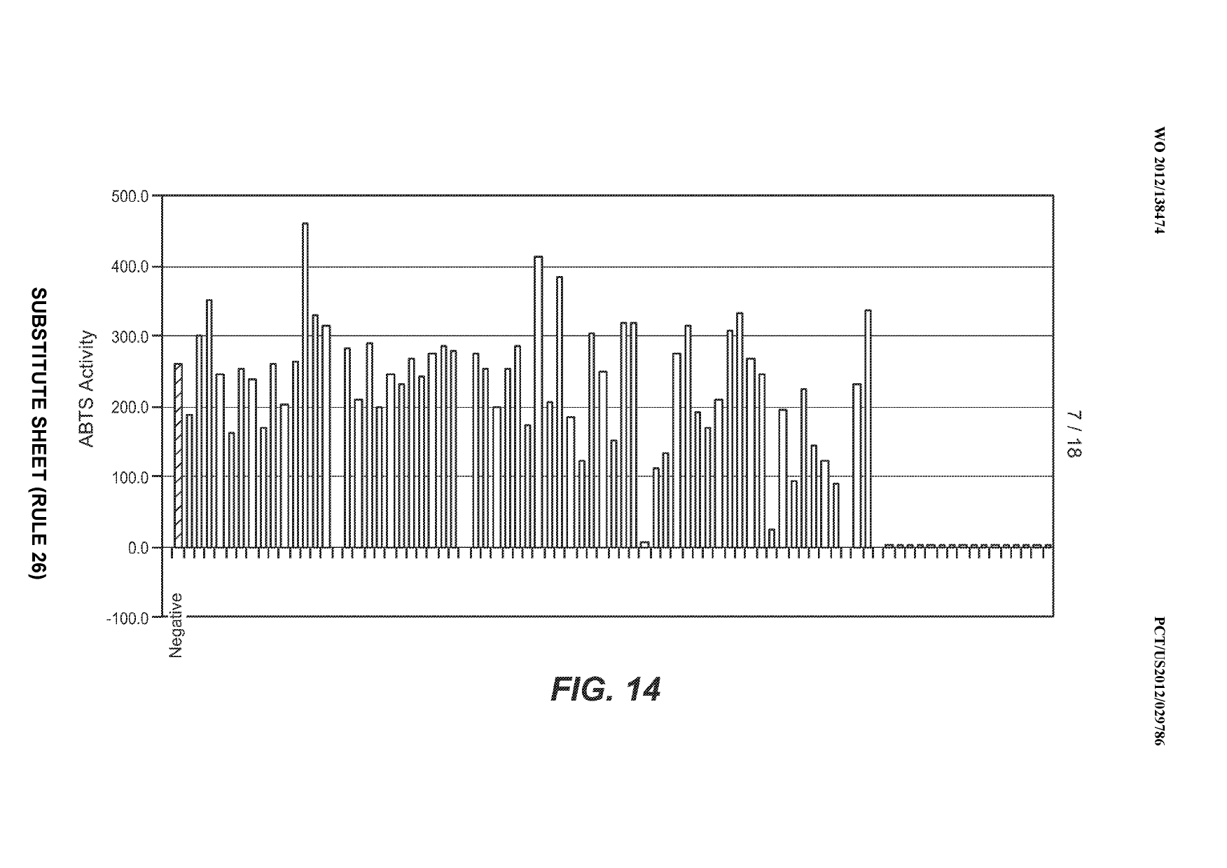

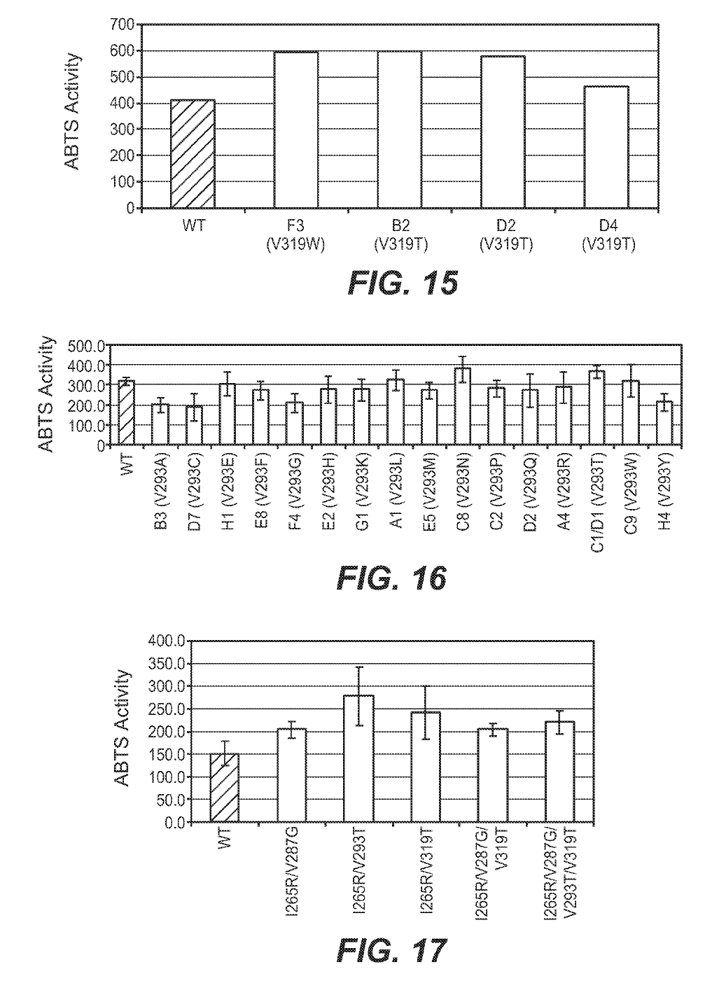

65 transformants were selected and transferred to a 96-well MTP filled with 0.2 ml of PDA containing 1.2 mg/ml 5-FOA and 0.5 mg/ml uridine using a colony picker (CP-7200, Norgren Systems). The MTP was incubated at 28° C. for 2 days and left at room temperature for 3 days to promote sporulation. All clones were transferred to a 96-well filter plate, which was incubated for 5 days. An ABTS assay was performed as Example 2. FIG. 14 shows the ABTS activity for all 65 transformants. The four transformants showing higher ABTS activity were selected for further analysis, and the mutations were identified as in Example 4. FIG. 15 shows the variants that produced the highest laccase expression or activity, i.e., V319W and V319T.

### Example 7

### Site Evaluation Library #4 (SEL4) Variants

A non-conservative, hydrophobic amino acid residue (V293) located on the surface of Cerrena laccase was selected for further engineering. Briefly, pairs of complementary primers overlapping the V293 codon were prepared and used to introduce amino acid residue changes. The SEL4 library variants were generated in same way as the SEL1 variants in Example 4 DNA from E. coli colonies were subjected to DNA sequence analysis. DNA from each of 16 different variants was used as template in separate PCR reactions. Three tubes of 100 μl of PCR reactions were performed for each variant, and the resulting 16 different PCR fragments were separately transformed into the Archy3 strain of T. reesei as in Example 4.

Four transformants corresponding to each variant were selected and transferred to a 96-well MTP filled with 0.2 ml of PDA containing 1.2 mg/ml 5-FOA and 0.5 mg/ml uridine. The MTP was incubated at 28° C. for 1 days and left at room temperature for 3 days to promote sporulation. All clones were transferred to a 96-well filter plate, which was incubated for 5 days. The ABTS assay was performed as in Example 2. FIG. 16 showed the ABTS activity for all the transformants screened. The results indicate that two variants (V293N and V293T) demonstrated higher laccase expression or activity than the wild type control.

### Example 8

### Combinatorial Variants

Three mutations (i.e., V287G, V293T, and V319T) were selected for combination with mutation I265R. Three primers (i.e., the V287G reverse primer, the V293T forward primer, and the V319 forward primer) were prepared and used to introduce amino acid residue changes at all three position and to generate all possible combinations that include the I265R mutation. As in Example 4, a single PCR reaction was used. Five variants were obtained, i.e., I265R/V287G, I265R/V293T, I265R/V319T, I265R/V287G/V319T, and I265R/V287G/V293T/V319T.

Plasmid DNA corresponding to each of the five different variants was then used as a template for PCR. Three tubes of 100 μl PCR reactions were prepared using each template, and each of the five resulting PCR fragments was transformed the Archy3 strain of T. reesei as in Example 2. Six transformants from each variant were picked to a 96-well MTP filled with 0.2 ml of PDA containing 1.2 mg/ml 5-FOA and 0.5 mg/ml uridine. The MTP was incubated at 28° C. for over 1 day and left at room temperature for 2 days to promote sporulation. Spores were transferred to a 96-well filter plate and incubated for 5 days at 28° C. An ABTS assay was performed as in Example 2. FIG. 17 shows the ABTS activity assay for all transformants screened. The results indicate that combinations of mutations produced laccase variants having greater expression and/or specific activity that the wild-type laccase.

Four additional mutations (F68L, V287P, N335R, and N130E) were also selected for combination with mutation I265R. Four pairs of primers were prepared and used to introduce the indicated amino acid residue changes in combination with variant I265R, as in Example 4. Four variants were obtained, i.e., I265R/V287P, F68L/I265R, I265R/N335R, and I265R/N130E. Plasmid DNA corresponding to each of the four different variants was then used as template for PCR. Three tubes of 100-μl PCR reactions were prepared using each template, and each of the four resulting PCR fragments was transformed the Archy3 strain of T. reesei as in Example 2. Six transformants from each variant were picked to a 96-well MTP filled with 0.2 ml of PDA containing 1.2 mg/ml 5-FOA and 0.5 mg/ml uridine. The MTP was incubated at 28° C. for over 1 day and left at room temperature for more than 2 days to promote sporulation. Spores were transferred to a 96-well filter plate and incubated for 5 days at 28° C. An ABTS assay was performed as in Example 2.

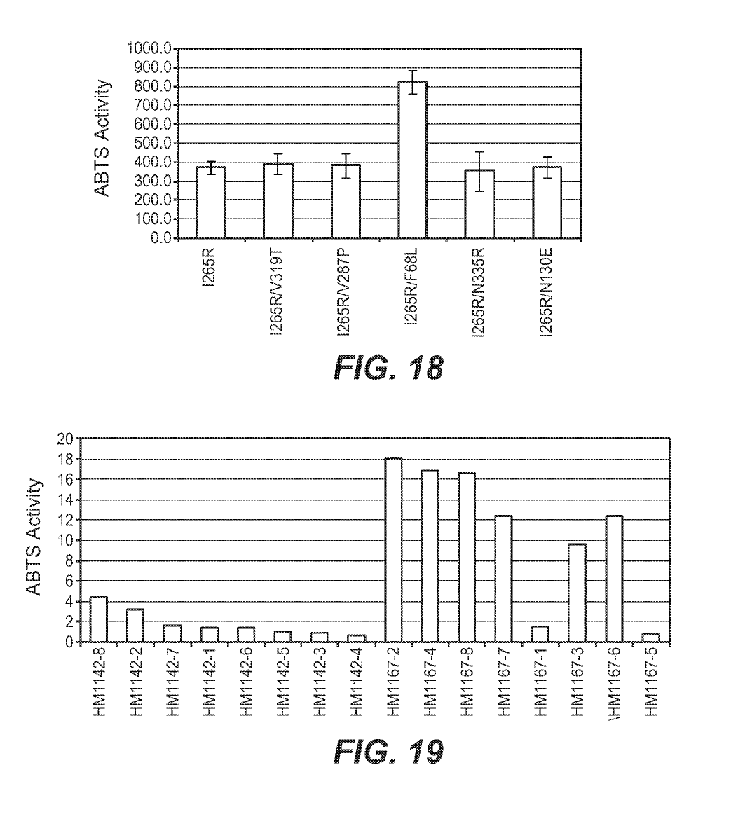

FIG. 18 shows the ABTS activity assay for all the transformants screened. The results indicate that the laccase variant having the combination of the F68L and I265R mutations had much greater expression and/or specific activity that the wild-type laccase, or other variants tested. Plasmid DNA encoding the F68L/I265R laccase variant and plasmid DNA encoding the wild-type laccase were separately transformed into T. reesei cell using biolistic transformation. A total of 12 stable F68L/I265R transformants (i.e., the “67” clones) and 14 wild-type transformants (i.e., the “42” clones) were obtained. 8 stable transformants of each type were grown in shake flasks and tested for laccase activity. As shown in FIG. 19, the variant laccase (i.e., the “67” clones) demonstrated more than a 4-fold increases in expression and/or specific activity compared to the wild type laccase (i.e., the “42” clones).

1. A variant laccase enzyme derived from a parental laccase enzyme, the variant laccase enzyme having:
(a) a mutation at a position corresponding to position 68 of the amino acid sequence of SEQ ID NO: 11;
(b) a mutation that alters the surface charge of the parental laccase enzyme;
(c) a mutation that alters the surface hydrophobicity of the parental laccase enzyme; or
(d) a mutation at an amino acid position corresponding to a non-conservative, hydrophobic amino acid residue located on the surface of the parental laccase enzyme;
wherein the mutation is a substitution to a different amino acid residue compared to the parental laccase.

2. The variant laccase enzyme of claim 1, having a mutation at a position corresponding to position 68 of the amino acid sequence of SEQ ID NO: 11, wherein the mutation is a substitution of an aromatic amino acid residue to a non-aromatic amino acid residue.

3. The variant laccase enzyme of claim 2, wherein the mutation is a substitution of an aromatic amino acid residue to an aliphatic amino acid residue.

4. The variant laccase enzyme of claim 3, wherein the mutation is a substitution of an aromatic amino acid residue to A, V, L, or I.

5. The variant laccase enzyme of claim 4, wherein the mutation is equivalent to F68L in SEQ ID NO: 11.

6. The variant laccase enzyme of claim 1, having a mutation that alters the surface charge or alters the surface hydrophobicity of the parental laccase enzyme, wherein the mutation is at a position equivalent to position 130, 265, 287, 293, or 319, in SEQ ID NO: 11.

7. The variant laccase enzyme of claim 1, having a mutation that alters the surface charge or alters the surface hydrophobicity of the parental laccase enzyme, wherein the mutation is at a position equivalent to position 130 in SEQ ID NO: 11.

8. The variant laccase enzyme of claim 1, having a mutation that alters the surface charge or alters the surface hydrophobicity of the parental laccase enzyme, wherein the mutation is at:
(a) an amino acid position equivalent to position 130 in SEQ ID NO: 11, wherein the residue in the parental laccase is substituted with a different residue selected from D, E, R, and K;
(b) an amino acid position equivalent to position 265 in SEQ ID NO: 11, wherein the residue in the parental laccase is substituted with a different residue selected from R, H, and V;
(c) an amino acid position equivalent to position 287 in SEQ ID NO: 11, wherein the residue in the parental laccase is substituted with a different residue selected from P, H, and G;
(d) an amino acid position equivalent to position 293 in SEQ ID NO: 11, wherein the residue in the parental laccase is substituted with a different residue selected from N, T, and S; or
(e) an amino acid position equivalent to position 319 in SEQ ID NO: 11, wherein the residue in the parental laccase is substituted with a different residue selected from W, T, and S.

9. The variant laccase enzyme of claim 1, having mutations equivalent to:
(a) I265R/V287G,
(b) I265R/V293T;
(c) I265R/V319T;
(d) I265R/V287G/V319T;
(e) I265R/V287G/V293T/V319T;
(f) I265R/V287P;
(g) I265R/N335R;
(h) I265R/N130E;
(i) F68L/I265R;
(j) F68L/I265R/V287G;
(k) F68L/I265R/V293T;
(l) F68L/I265R/V319T;
(m) F68L/I265R/V287G/V319T;
(n) F68L/I265R/V287G/V293T/V319T;
(O) F68L/I265R/V287P;
(p) F68L/I265R/N335R; or
(q) F68L/I265R/N130E;
in SEQ ID NO: 11.

10. The variant laccase enzyme of claim 1, wherein the parental laccase is obtainable from a Cerrena species.

11. The variant laccase enzyme of claim 1, wherein the parental laccase is obtainable from Cerrena unicolor.

12. The variant laccase enzyme of claim 1, wherein the parental laccase is laccase D from C. unicolor.

13. The variant laccase enzyme of claim 1, wherein the parental laccase has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

14. The variant laccase enzyme of claim 1, having an amino acid sequence that is at least 70% identical to the amino acid sequence of SEQ ID NO: 11.

15. The variant laccase enzyme of claim 1, having an amino acid sequence that is at least 80% identical to the amino acid sequence of SEQ ID NO: 11.

16. The variant laccase enzyme of claim 1, having an amino acid sequence that is at least 90% identical to the amino acid sequence of SEQ ID NO: 11.

17. The variant laccase enzyme of claim 1, having an amino acid sequence that is at least 95% identical to the amino acid sequence of SEQ ID NO: 11.

18. The variant laccase enzyme of claim 1, further comprising a mutation that introduces a glycosylation site into the amino acid sequence of the parental laccase.

19. A composition comprising the variant laccase of claim 1.

20. The composition of claim 19, further comprising a chemical mediator.

21. The composition of claim 20, wherein the chemical mediator is a phenolic compound.

22. The composition of claim 21, wherein the chemical mediator is a phenolic compound is selected from the group consisting of syringonitrile, acetosyringone, and methyl syringate.

23. A method of bleaching a surface comprising contacting the surface with a composition of any of the preceding claims.
